SEQUENCE LISTING (38 sequences)

SEQ ID NO: 1 - DNA, 268 bp, Homo sapiens
  source 1..268: mol_type = unassigned DNA
tcggaccgcggcaactacacctgcgtcgtggagaacaagtttggcagcatccggcagacgtacacgctggacgtgctggagtgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctca

SEQ ID NO: 2 - DNA, 378 bp, Homo sapiens
  source 1..378: mol_type = unassigned DNA
gaccgcggcaactacacctgcgtcgtggagaacaagtttggcagcatccggcagacgtacacgctggacgtgctgggtgagggccctggggcggcgcgggggtgggggcggcagtggcggtggtggtgagggagggggtggcccctgagcgtcatctgcccccacagagcgctgcccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaaggtgggccaccgtgtgcacgt

SEQ ID NO: 3 - DNA, 234 bp, Homo sapiens
  source 1..234: mol_type = unassigned DNA
gcgggcaattctattgggttttctcatcactctgcgtggctggtggtgctgccagccgaggaggagctggtggaggctgacgaggcgtgcagtgtgtatgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccg

SEQ ID NO: 4 - DNA, 301 bp, Homo sapiens
  source 1..301: mol_type = unassigned DNA
ctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtgtgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccgctcaagc

SEQ ID NO: 5 - DNA, 21 bp, synthetic construct
  source 1..21: mol_type = other DNA
gacctggaccgtgtccttacc

SEQ ID NO: 6 - DNA, 21 bp, synthetic construct
  source 1..21: mol_type = other DNA
cttccccagttccaggttctt

SEQ ID NO: 7 - DNA, 20 bp, synthetic construct
  source 1..20: mol_type = other DNA
aggacctggaccgtgtcctt

SEQ ID NO: 8 - DNA, 20 bp, synthetic construct
  source 1..20: mol_type = other DNA
tataggtccggtggacaggg

SEQ ID NO: 9 - DNA, 20 bp, synthetic construct
  source 1..20: mol_type = other DNA
ctggaccgtgtccttaccgt

SEQ ID NO: 10 - DNA, 20 bp, synthetic construct
  source 1..20: mol_type = other DNA
gcagcccaggattgaactgt

SEQ ID NO: 11 - DNA, 23 bp, synthetic construct
  source 1..23: mol_type = other DNA
tggatcgaattctcactctcaca

SEQ ID NO: 12 - DNA, 21 bp, synthetic construct
  source 1..21: mol_type = other DNA
gccaagcaatctgcgtatttg

SEQ ID NO: 13 - DNA, 23 bp, synthetic construct
  source 1..23: mol_type = other DNA
gctcttcaatacagccctgatca

SEQ ID NO: 14 - DNA, 24 bp, synthetic construct
  source 1..24: mol_type = other DNA
acttggatcgaattctcactctca

SEQ ID NO: 15 - DNA, 23 bp, synthetic construct
  source 1..23: mol_type = other DNA
tggatcgaattctcactctcaca

SEQ ID NO: 16 - DNA, 25 bp, synthetic construct
  source 1..25: mol_type = other DNA
gcaaagcctgaattttcttgaataa

SEQ ID NO: 17 - DNA, 18 bp, synthetic construct
  source 1..18: mol_type = other DNA
gcatccggcagacgtaca

SEQ ID NO: 18 - DNA, 15 bp, synthetic construct
  source 1..15: mol_type = other DNA
ccccgcctgcaggat

SEQ ID NO: 19 - DNA, 18 bp, synthetic construct
  source 1..18: mol_type = other DNA
gcatccggcagacgtaca

SEQ ID NO: 20 - DNA, 15 bp, synthetic construct
  source 1..15: mol_type = other DNA
ccccgcctgcaggat

SEQ ID NO: 21 - DNA, 19 bp, synthetic construct
  source 1..19: mol_type = other DNA
aggagctggtggaggctga

SEQ ID NO: 22 - DNA, 19 bp, synthetic construct
  source 1..19: mol_type = other DNA
ccgtagctgaggatgcctg

SEQ ID NO: 23 - DNA, 18 bp, synthetic construct
  source 1..18: mol_type = other DNA
ctggtggaggctgacgag

SEQ ID NO: 24 - DNA, 16 bp, synthetic construct
  source 1..16: mol_type = other DNA
agcccaccccgtagct

SEQ ID NO: 25 - DNA, 21 bp, synthetic construct
  source 1..21: mol_type = other DNA
gtcgtggagaacaagtttggc

SEQ ID NO: 26 - DNA, 17 bp, synthetic construct
  source 1..17: mol_type = other DNA
gtctggttggccggcag

SEQ ID NO: 27 - DNA, 21 bp, synthetic construct
  source 1..21: mol_type = other DNA
gtcgtggagaacaagtttggc

SEQ ID NO: 28 - DNA, 17 bp, synthetic construct
  source 1..17: mol_type = other DNA
gtctggttggccggcag

SEQ ID NO: 29 - DNA, 19 bp, synthetic construct
  source 1..19: mol_type = other DNA
aggagctggtggaggctga

SEQ ID NO: 30 - DNA, 19 bp, synthetic construct
  source 1..19: mol_type = other DNA
ccgtagctgaggatgcctg

SEQ ID NO: 31 - DNA, 16 bp, synthetic construct
  source 1..16: mol_type = other DNA
gacgaggcgggcagtg

SEQ ID NO: 32 - DNA, 19 bp, synthetic construct
  source 1..19: mol_type = other DNA
gaagaagcccaccccgtag

SEQ ID NO: 33 - DNA, 2850 bp, synthetic construct
  source 1..2850: mol_type = other DNA
atgggcgcccctgcctgcgccctcgcgctctgcgtggccgtggccatcgtggccggcgcctcctcggagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagacggcgggcgctaacaccaccgacaaggagctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtatgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccgctcaagcgacaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacgaccaacggccggctgcccgtgaagtggatggcgcctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctcccagaggcccaccttcaagcagctggtggaggacctggaccgtgtccttaccgtgacgtccaccgacgtaaaggcgacacaggaggagaaccgggagctgaggagcaggtgtgaggagctccacgggaagaacctggaactggggaagatcatggacaggttcgaagaggttgtgtaccaggccatggaggaagttcagaagcagaaggaactttccaaagctgaaatccagaaagttctaaaagaaaaagaccaacttaccacagatctgaactccatggagaagtccttctccgacctcttcaagcgttttgagaaacagaaagaggtgatcgagggctaccgcaagaacgaagagtcactgaagaagtgcgtggaggattacctggcaaggatcacccaggagggccagaggtaccaagccctgaaggcccacgcggaggagaagctgcagctggcaaacgaggagatcgcccaggtccggagcaaggcccaggcggaagcgttggccctccaggccagcctgaggaaggagcagatgcgcatccagtcgctggagaagacagtggagcagaagactaaagagaacgaggagctgaccaggatctgcgacgacctcatctccaagatggagaagatctga

SEQ ID NO: 34 - DNA, 2955 bp, synthetic construct
  source 1..2955: mol_type = other DNA
atgggcgcccctgcctgcgccctcgcgctctgcgtggccgtggccatcgtggccggcgcctcctcggagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagacggcgggcgctaacaccaccgacaaggagctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtatgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccgctcaagcgacaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacgaccaacggccggctgcccgtgaagtggatggcgcctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctcccagaggcccaccttcaagcagctggtggaggacctggaccgtgtccttaccgtgacgtccaccgacgtgccaggcccacccccaggtgttcccgcgcctgggggcccacccctgtccaccggacctatagtggacctgctccagtacagccagaaggacctggatgcagtggtaaaggcgacacaggaggagaaccgggagctgaggagcaggtgtgaggagctccacgggaagaacctggaactggggaagatcatggacaggttcgaagaggttgtgtaccaggccatggaggaagttcagaagcagaaggaactttccaaagctgaaatccagaaagttctaaaagaaaaagaccaacttaccacagatctgaactccatggagaagtccttctccgacctcttcaagcgttttgagaaacagaaagaggtgatcgagggctaccgcaagaacgaagagtcactgaagaagtgcgtggaggattacctggcaaggatcacccaggagggccagaggtaccaagccctgaaggcccacgcggaggagaagctgcagctggcaaacgaggagatcgcccaggtccggagcaaggcccaggcggaagcgttggccctccaggccagcctgaggaaggagcagatgcgcatccagtcgctggagaagacagtggagcagaagactaaagagaacgaggagctgaccaggatctgcgacgacctcatctccaagatggagaagatctga

SEQ ID NO: 35 - DNA, 3765 bp, synthetic construct
  source 1..3765: mol_type = other DNA
atgggcgcccctgcctgcgccctcgcgctctgcgtggccgtggccatcgtggccggcgcctcctcggagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagtcctggatcagtgagagtgtggaggccgacgtgcgcctccgcctggccaatgtgtcggagcgggacgggggcgagtacctctgtcgagccaccaatttcataggcgtggccgagaaggccttttggctgagcgttcacgggccccgagcagccgaggaggagctggtggaggctgacgaggcgggcagtgtgtatgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccgctcaagcgacaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacgaccaacggccggctgcccgtgaagtggatggcgcctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctcccagaggcccaccttcaagcagctggtggaggacctggaccgtgtccttaccgtgacgtccaccgacaatgttatggaacagttcaatcctgggctgcgaaatttaataaacctggggaaaaattatgagaaagctgtaaacgctatgatcctggcaggaaaagcctactacgatggagtggccaagatcggtgagattgccactgggtcccccgtgtcaactgaactgggacatgtcctcatagagatttcaagtacccacaagaaactcaacgagagtcttgatgaaaattttaaaaaattccacaaagagattatccatgagctggagaagaagatagaacttgacgtgaaatatatgaacgcaactctaaaaagataccaaacagaacacaagaataaattagagtctttggagaaatcccaagctgagttgaagaagatcagaaggaaaagccaaggaagccgaaacgcactcaaatatgaacacaaagaaattgagtatgtggagaccgttacttctcgtcagagtgaaatccagaaattcattgcagatggttgcaaagaggctctgcttgaagagaagaggcgcttctgctttctggttgataagcactgtggctttgcaaaccacatacattattatcacttacagtctgcagaactactgaattccaagctgcctcggtggcaggagacctgtgttgatgccatcaaagtgccagagaaaatcatgaatatgatcgaagaaataaagaccccagcctctacccccgtgtctggaactcctcaggcttcacccatgatcgagagaagcaatgtggttaggaaagattacgacaccctttctaaatgctcaccaaagatgccccccgctccttcaggcagagcatataccagtcccttgatcgatatgtttaataacccagccacggctgccccgaattcacaaagggtaaataattcaacaggtacttccgaagatcccagtttacagcgatcagtttcggttgcaacgggactgaacatgatgaagaagcagaaagtgaagaccatcttcccgcacactgcgggctccaacaagaccttactcagctttgcacagggagatgtcatcacgctgctcatccccgaggagaaggatggctggctctatggagaacacgacgtgtccaaggcgaggggttggttcccgtcgtcgtacacgaagttgctggaagaaaatgagacagaagcagtgaccgtgcccacgccaagccccacaccagtgagaagcatcagcaccgtgaacttgtctgagaatagcagtgttgtcatccccccacccgactacttggaatgcttgtccatgggggcagctgccgacaggagagcagattcggccaggacgacatccacctttaaggccccagcgtccaagcccgagaccgcggctcctaacgatgccaacgggactgcaaagccgccttttctcagcggagaaaacccctttgccactgtgaaactccgcccgactgtgacgaatgatcgctcggcacccatcattcgatga

SEQ ID NO: 36 - DNA, 4989 bp, synthetic construct
  source 1..4989: mol_type = other DNA
atggtcagctggggtcgtttcatctgcctggtcgtggtcaccatggcaaccttgtccctggcccggccctccttcagtttagttgaggataccacattagagccagaagagccaccaaccaaataccaaatctctcaaccagaagtgtacgtggctgcgccaggggagtcgctagaggtgcgctgcctgttgaaagatgccgccgtgatcagttggactaaggatggggtgcacttggggcccaacaataggacagtgcttattggggagtacttgcagataaagggcgccacgcctagagactccggcctctatgcttgtactgccagtaggactgtagacagtgaaacttggtacttcatggtgaatgtcacagatgccatctcatccggagatgatgaggatgacaccgatggtgcggaagattttgtcagtgagaacagtaacaacaagagagcaccatactggaccaacacagaaaagatggaaaagcggctccatgctgtgcctgcggccaacactgtcaagtttcgctgcccagccggggggaacccaatgccaaccatgcggtggctgaaaaacgggaaggagtttaagcaggagcatcgcattggaggctacaaggtacgaaaccagcactggagcctcattatggaaagtgtggtcccatctgacaagggaaattatacctgtgtagtggagaatgaatacgggtccatcaatcacacgtaccacctggatgttgtggagcgatcgcctcaccggcccatcctccaagccggactgccggcaaatgcctccacagtggtcggaggagacgtagagtttgtctgcaaggtttacagtgatgcccagccccacatccagtggatcaagcacgtggaaaagaacggcagtaaatacgggcccgacgggctgccctacctcaaggttctcaaggccgccggtgttaacaccacggacaaagagattgaggttctctatattcggaatgtaacttttgaggacgctggggaatatacgtgcttggcgggtaattctattgggatatcctttcactctgcatggttgacagttctgccagcgcctggaagagaaaaggagattacagcttccccagactacctggagatagccatttactgcataggggtcttcttaatcgcctgtatggtggtaacagtcatcctgtgccgaatgaagaacacgaccaagaagccagacttcagcagccagccggctgtgcacaagctgaccaaacgtatccccctgcggagacaggtaacagtttcggctgagtccagctcctccatgaactccaacaccccgctggtgaggataacaacacgcctctcttcaacggcagacacccccatgctggcaggggtctccgagtatgaacttccagaggacccaaaatgggagtttccaagagataagctgacactgggcaagcccctgggagaaggttgctttgggcaagtggtcatggcggaagcagtgggaattgacaaagacaagcccaaggaggcggtcaccgtggccgtgaagatgttgaaagatgatgccacagagaaagacctttctgatctggtgtcagagatggagatgatgaagatgattgggaaacacaagaatatcataaatcttcttggagcctgcacacaggatgggcctctctatgtcatagttgagtatgcctctaaaggcaacctccgagaatacctccgagcccggaggccacccgggatggagtactcctatgacattaaccgtgttcctgaggagcagatgaccttcaaggacttggtgtcatgcacctaccagctggccagaggcatggagtacttggcttcccaaaaatgtattcatcgagatttagcagccagaaatgttttggtaacagaaaacaatgtgatgaaaatagcagactttggactcgccagagatatcaacaatatagactattacaaaaagaccaccaatgggcggcttccagtcaagtggatggctccagaagccctgtttgatagagtatacactcatcagagtgatgtctggtccttcggggtgttaatgtgggagatcttcactttagggggctcgccctacccagggattcccgtggaggaactttttaagctgctgaaggaaggacacagaatggataagccagccaactgcaccaacgaactgtacatgatgatgagggactgttggcatgcagtgccctcccagagaccaacgttcaagcagttggtagaagacttggatcgaattctcactctcacaaccaatgagatcatggaggaaacaaatacgcagattgcttggccatcaaaactgaagatcggagccaaatccaagaaagatccccatattaaggtttctggaaagaaagaagatgttaaagaagccaaggaaatgatcatgtctgtcttagacacaaaaagcaatcgagtcacactgaagatggatgtttcacatacagaacattcacatgtaatcggcaaaggtggcaacaatattaaaaaagtgatggaagaaaccggatgccatatccactttccagattccaacaggaataaccaagcagaaaaaagcaaccaggtatctatagcgggacaaccagcaggagtagaatctgcccgagttagaattcgggagctgcttcctttggtgctgatgtttgagctaccaattgctggaattcttcaaccggttcctgatcctaattccccctctattcagcatatatcacaaacgtacaatatttcagtatcatttaaacagcgttcccgaatgtatggtgctactgtcatagtacgagggtctcagaataacactagtgctgtgaaggaaggaactgccatgctgttagaacatcttgctgggagcttagcatcagctattcctgtgagcacacaactagatattgcagctcaacatcatctctttatgatgggtcgaaatgggagcaacatcaaacatatcatgcagagaacaggtgctcagatccactttcctgatcccagtaatccacaaaagaaatctaccgtctacctccagggcaccattgagtctgtctgtcttgcaaggcaatatctcatgggttgtcttcctcttgtgttgatgtttgatatgaaggaagaaattgaagtagatccacaattcattgcgcagttgatggaacagcttgatgtcttcatcagtattaaaccaaagcccaaacagccaagcaagtctgtgattgtgaaaagtgttgagcgaaatgccttaaatatgtatgaagcaaggaaatgtctcctcggacttgaaagcagtggggttaccatagcaaccagtccatccccagcatcctgccctgccggcctggcatgtcccagcctggatatcttagcttcagcaggccttggactcactggactaggtcttttgggacccaccaccttatctctgaacacttcaacaaccccaaactcactcttgaatgctcttaatagctcagtcagtcctttgcaaagtccaagttctggtacacccagccccacattatgggcacccccacttgctaatacttcaagtgccacaggtttttctgctataccacaccttatgattccatctactgcccaagccacattaactaatattttgttgtctggagtgcccacctatgggcacacagctccatctccccctcctggcttgactcctgttgatgtccatatcaacagtatgcagaccgaaggcaaaaaaatctctgctgctttaaatggacatgcacagtctccagatataaaatatggtgcaatatccacttcatcacttggagaaaaagtgctgagtgcaaatcacggggatccgtccatccagacaagtgggtctgagcagacatctcccaaatcaagccccactgaaggttgtaatgatgcttttgttgaagtaggcatgcctcgaagtccttcccattctgggaatgctggtgacttgaaacagatgatgtgtccctccaaggtttcctgtgccaaaaggcagacagtggaactattgcaaggcacgaaaaactcacacttacacagcactgacaggttgctctcagaccctgaactgagtgctaccgaaagccctttggctgacaagaaggctccagggagtgagcgcgctgcagagagggcagcagctgcccagcaaaactccgaaagggcccaccttgctccacggtcatcatatgtcaacatgcaggcatttgactatgaacagaagaagctattagccaccaaagctatgttaaagaaaccagtggtgacggaggtcagaacgcccacaaatacctggagtggcctgggtttttctaaatccatgccagctgaaactatcaaggagttgagaagggccaatcatgtgtcctataagcccacaatgacaaccacttatgagggctcatccatgtccctttcacggtccaacagtcgtgagcacttgggaggtggaagcgaatctgataactggagagaccgaaatggaattggacctggaagtcatagtgaatttgcagcttctattggcagccctaagcgtaaacaaaacaaatcaacggaacactatctcagcagtagcaattacatggactgcatttcctcgctgacaggaagcaatggctgtaacttaaatagctctttcaaaggttctgacctccctgagctcttcagcaaactgggcctgggcaaatacacagatgttttccagcaacaagagatcgatcttcagacattcctcactctcacagatcaggatctgaaggagctgggaataactacttttggtgccaggaggaaaatgctgcttgcaatttcagaactaaataaaaaccgaagaaagctttttgaatcgccaaatgcacgcacctctttcctggaaggtggagcgagtggaaggctaccccgtcagtatcactcagacattgctagtgtcagtggccgctggtag

SEQ ID NO: 37 - DNA, 3213 bp, synthetic construct
  source 1..3213: mol_type = other DNA
atggtcagctggggtcgtttcatctgcctggtcgtggtcaccatggcaaccttgtccctggcccggccctccttcagtttagttgaggataccacattagagccagaagagccaccaaccaaataccaaatctctcaaccagaagtgtacgtggctgcgccaggggagtcgctagaggtgcgctgcctgttgaaagatgccgccgtgatcagttggactaaggatggggtgcacttggggcccaacaataggacagtgcttattggggagtacttgcagataaagggcgccacgcctagagactccggcctctatgcttgtactgccagtaggactgtagacagtgaaacttggtacttcatggtgaatgtcacagatgccatctcatccggagatgatgaggatgacaccgatggtgcggaagattttgtcagtgagaacagtaacaacaagagagcaccatactggaccaacacagaaaagatggaaaagcggctccatgctgtgcctgcggccaacactgtcaagtttcgctgcccagccggggggaacccaatgccaaccatgcggtggctgaaaaacgggaaggagtttaagcaggagcatcgcattggaggctacaaggtacgaaaccagcactggagcctcattatggaaagtgtggtcccatctgacaagggaaattatacctgtgtagtggagaatgaatacgggtccatcaatcacacgtaccacctggatgttgtggagcgatcgcctcaccggcccatcctccaagccggactgccggcaaatgcctccacagtggtcggaggagacgtagagtttgtctgcaaggtttacagtgatgcccagccccacatccagtggatcaagcacgtggaaaagaacggcagtaaatacgggcccgacgggctgccctacctcaaggttctcaaggccgccggtgttaacaccacggacaaagagattgaggttctctatattcggaatgtaacttttgaggacgctggggaatatacgtgcttggcgggtaattctattgggatatcctttcactctgcatggttgacagttctgccagcgcctggaagagaaaaggagattacagcttccccagactacctggagatagccatttactgcataggggtcttcttaatcgcctgtatggtggtaacagtcatcctgtgccgaatgaagaacacgaccaagaagccagacttcagcagccagccggctgtgcacaagctgaccaaacgtatccccctgcggagacaggtaacagtttcggctgagtccagctcctccatgaactccaacaccccgctggtgaggataacaacacgcctctcttcaacggcagacacccccatgctggcaggggtctccgagtatgaacttccagaggacccaaaatgggagtttccaagagataagctgacactgggcaagcccctgggagaaggttgctttgggcaagtggtcatggcggaagcagtgggaattgacaaagacaagcccaaggaggcggtcaccgtggccgtgaagatgttgaaagatgatgccacagagaaagacctttctgatctggtgtcagagatggagatgatgaagatgattgggaaacacaagaatatcataaatcttcttggagcctgcacacaggatgggcctctctatgtcatagttgagtatgcctctaaaggcaacctccgagaatacctccgagcccggaggccacccgggatggagtactcctatgacattaaccgtgttcctgaggagcagatgaccttcaaggacttggtgtcatgcacctaccagctggccagaggcatggagtacttggcttcccaaaaatgtattcatcgagatttagcagccagaaatgttttggtaacagaaaacaatgtgatgaaaatagcagactttggactcgccagagatatcaacaatatagactattacaaaaagaccaccaatgggcggcttccagtcaagtggatggctccagaagccctgtttgatagagtatacactcatcagagtgatgtctggtccttcggggtgttaatgtgggagatcttcactttagggggctcgccctacccagggattcccgtggaggaactttttaagctgctgaaggaaggacacagaatggataagccagccaactgcaccaacgaactgtacatgatgatgagggactgttggcatgcagtgccctcccagagaccaacgttcaagcagttggtagaagacttggatcgaattctcactctcacaaccaatgagatggcagatgatcagggctgtattgaagagcagggggttgaggattcagcaaatgaagattcagtggatgctaagccagaccggtcctcgtttgtaccgtccctcttcagtaagaagaagaaaaatgtcaccatgcgatccatcaagaccacccgggaccgagtgcctacatatcagtacaacatgaattttgaaaagctgggcaaatgcatcataataaacaacaagaactttgataaagtgacaggtatgggcgttcgaaacggaacagacaaagatgccgaggcgctcttcaagtgcttccgaagcctgggttttgacgtgattgtctataatgactgctcttgtgccaagatgcaagatctgcttaaaaaagcttctgaagaggaccatacaaatgccgcctgcttcgcctgcatcctcttaagccatggagaagaaaatgtaatttatgggaaagatggtgtcacaccaataaaggatttgacagcccactttaggggggatagatgcaaaacccttttagagaaacccaaactcttcttcattcaggcttgccgagggaccgagcttgatgatggcatccaggccgactcggggcccatcaatgacacagatgctaatcctcgatacaagatcccagtggaagctgacttcctcttcgcctattccacggttccaggctattactcgtggaggagcccaggaagaggctcctggtttgtgcaagccctctgctccatcctggaggagcacggaaaagacctggaaatcatgcagatcctcaccagggtgaatgacagagttgccaggcactttgagtctcagtctgatgacccacacttccatgagaagaagcagatcccctgtgtggtctccatgctcaccaaggaactctacttcagtcaatag

SEQ ID NO: 38 - DNA, 24 bp, synthetic construct
  source 1..24: mol_type = other DNA
tttttttttttttttttttttttt

(19) United States
(12) Patent Application Publication
(10) Pub. No.: US 2025/0041574 A1
(43) Pub. Date: Feb. 6, 2025

(54) ERDAFITINIB FORMULATIONS AND OSMOTIC SYSTEMS FOR INTRAVESICAL ADMINISTRATION

(71) Applicant: TARIS Biomedical LLC, Lexington, MA (US)

(72) Inventors: Karen DANIEL, Lexington, MA (US); Jens Julien Maurits André DHONDT, Beerse (BE); Kristof Johan W DUBOIS, Beerse (BE); Philip Erna H HEYNS, Beerse (BE); Kristof Leonard KIMPE, Beerse (BE); Srinivas MAMIDI, Raritan, NJ (US); Dennis GIESING, Lexington, MA (US)

(21) Appl. No.: 18/806,440

(22) Filed: Aug. 15, 2024

Related U.S. Application Data
(63) Continuation of application No. PCT/US2023/062871, filed on Feb. 17, 2023.
(60) Provisional application No. 63/311,855, filed on Feb. 18, 2022.

(51) Int. Cl. A61M 31/00; A61K 9/00; A61K 9/20; A61K 31/498

(52) U.S. Cl. CPC A61M 31/002; A61K 9/0034; A61K 9/2027; A61K 9/2054; A61K 9/2095; A61K 31/498; A61M 2210/1085

(57) ABSTRACT

Provided herein are solid pharmaceutical compositions comprising an L-lactate salt of erdafitinib, processes for making such formulations, and drug delivery systems comprising such formulations, including systems for intravesical administration.

### CROSS-REFERENCE TO RELATED APPLICATIONS

This application claims priority to U.S. provisional application No. 63/311,855, filed Feb. 18, 2022, the contents of which are hereby incorporated by reference in its entirety.

### REFERENCE TO AN ELECTRONIC SEQUENCE LISTING

The contents of the electronic sequence listing (761662002140seqlisting.xml; Size: 53,009 bytes; and Date of Creation: Feb. 15, 2023) is herein incorporated by reference in its entirety.

### BACKGROUND

The present disclosure is generally in the field of pharmaceutical formulations and drug-device combination products, and more particularly relates to erdafitinib based formulations and systems for intravesical administration of such formulations.

Erdafitinib (N-(3,5-dimethoxyphenyl)-N′-(1-methylethyl)-N-[3-(1-methyl-1H-pyrazol-4-yl)quinoxalin-6-yl]ethane-1,2-diamine) is a potent pan FGFR kinase inhibitor that binds to and inhibits enzymatic activity of FGFR1, FGFR2, FGFR3 and FGFR4. Erdafitinib has been found to inhibit FGFR phosphorylation and signaling and decrease cell viability in cell lines expressing FGFR genetic alterations, including point mutations, amplifications, and fusions. Erdafitinib has demonstrated antitumor activity in FGFR-expressing cell lines and xenograft models derived from tumor types, including bladder cancer.

Currently, Erdafitinib (BALVERSA®) is available as film-coated tablets for oral administration, and is indicated for the treatment of adult patients with locally advanced or metastatic urothelial carcinoma that has susceptible fibroblast growth factor receptor FGFR3 or FGFR2 genetic alterations and progressed during or following at least one line of prior platinum-containing chemotherapy, including within 12 months of neoadjuvant or adjuvant platinum-containing chemotherapy.

U.S. Pat. No. 10,898,482 to Broggini and International Patent Application Publication No. WO 2020/201138 to De Porre describe certain erdafitinib formulations and treatment methods.

Examples of intravesical drug delivery systems are described in U.S. Pat. No. 8,679,094 to Cima et al., U.S. Pat. No. 9,017,312 to Lee et al., U.S. Pat. No. 9,107,816 to Lee et al., and U.S. Pat. No. 9,457,176 to Lee et al. In some embodiments, the intravesical systems include a water permeable housing defining a drug reservoir lumen which contains a solid or semi-solid drug formulation, and release of the drug in vivo occurs by water from the bladder diffusing into drug reservoir lumen to solubilize the drug, and then an osmotic pressure build-up in the drug reservoir lumen drives the solubilized drug out of the drug reservoir lumen through a release aperture.

U.S. Pat. No. 10,286,199 to Lee et al. discloses systems in which drug is released from a housing made of a first wall structure and a hydrophilic second wall structure, wherein the first wall structure is impermeable to the drug and the second wall structure is permeable to the drug. U.S. Pat. No. 10,894,150 to Lee also discloses systems in which drug is released from a housing made of a first wall structure that is impermeable to the drug and a second wall structure that is permeable to the drug.

### BRIEF SUMMARY

In one aspect, provided herein is an intravesical drug delivery system comprising an elongated body configured for intravesical insertion into a patient, and a drug formulation disposed in the elongated body, the drug formulation comprising erdaritinib (N-(3,5-dimethoxyphenyl)-N′-(1-methylethyl)-N-[3-(1-methyl-1H-pyrazol-4-yl)quinoxalin-6-yl]ethane-1,2-diamine) or a pharmaceutically acceptable salt thereof, in particular an L-lactate salt of erdafitinib (N-(3,5-dimethoxyphenyl)-N′-(1-methylethyl)-N-[3-(1-methyl-1H-pyrazol-4-yl)quinoxalin-6-yl]ethane-1,2-diamine). In some embodiments, the drug delivery system is configured to release erdafitinib from one or more openings in the elongated body, driven by osmotic pressure. In some embodiments, the erdafitinib is present as a mono L-lactate salt. In some embodiments, the elongated body comprises a biocompatible elastomer. In some embodiments, the biocompatible elastomer comprises silicone or a thermoplastic polyurethane. In some embodiments, the biocompatible elastomer comprises silicone. In some embodiments, the biocompatible elastomer comprises platinum cured silicone elastomer. In some embodiments, at least one of the one or more openings in the elongated body is located in a sidewall of the elongated body. In some embodiments, at least one of the one or more openings in the elongated body is located at a first end and/or at an opposing second end of the elongated body. In some embodiments, the drug delivery system has a single opening, which is located in a sidewall of the elongated body at position between a first end and an opposing second end of the elongated body. In some embodiments, the one or more openings has a diameter of between about 100 μm and about 200 μm. In some embodiments, the one or more openings has a diameter of about 150 μm.

In some embodiments, the drug delivery system is configured to release the erdafitinib by osmotic pressure through the one or more openings in the elongated body. In some embodiments, the elongated body comprises an annular wall structure defining a drug reservoir lumen in which the drug formulation is disposed. In some embodiments, the annular wall structure has a thickness of between about 0.1 mm to about 0.5 mm. In some embodiments, the annular wall structure has a thickness of about 0.2 mm.

In some embodiments, the drug delivery system further comprises a first end plug positioned at a first end of the annular wall structure, and a second end plug positioned at a second end of the annular wall structure. In some embodiments, the one or more openings in the elongated body of the drug delivery system comprise a single aperture in the annular wall structure, and the elongated body is configured to release the erdafitinib through the aperture. In some embodiments, the elongated body is configured to release the erdafitinib through microchannels transiently formed at one or both end regions of the annular wall structure. In some embodiments, the system is configured to release the erdafitinib at an average rate of 1 mg/day to 10 mg/day, in particular 1 mg/day to 10 mg/day of erdafitinib free base equivalent. In some embodiments, the system is configured to release the erdafitinib at an average rate of 1 mg/day to 6 mg/day. In some embodiments, the system is configured to release the erdafitinib at an average rate of 2 mg/day to 4 mg/day. In some embodiments, the system is configured to release the erdafitinib at an average rate of 4 mg/day. In some embodiments, the system is configured to release the erdafitinib at an average rate of 2 mg/day. In some embodiments, the system is configured to release the erdafitinib with a zero order release profile. In some embodiments, the system is configured to release the erdafitinib for up to about 30 days. In some embodiments, the system is configured to release the erdafitinib for up to about 90 days. In some embodiments, the system comprises 500 mg of the erdafitinib (free base equivalent).

In some embodiments, the drug delivery system is elastically deformable between a relatively straightened deployment shape suited for insertion through the urethra of a patient and into the patient's bladder and a retention shape suited to retain the system within the bladder. In some embodiments, the system is elastically deformable and comprises a tube having two opposing free ends, which are directed away from one another when the system is in a low-profile deployment shape and which are directed toward one another when the system is in a relatively expanded retention shape. In some embodiments, the system comprises an elastically deformable elongated body having two opposing free ends which lie within the boundaries of a bi-oval-like expanded retention shape. In some embodiments, the elongated body further comprises a retention frame lumen. In some embodiments, the system further comprises a nitinol wire disposed in the retention frame lumen.

In another aspect, provided herein is a pharmaceutical composition, or a drug delivery system comprising said pharmaceutical composition, wherein the pharmaceutical composition comprises a lactate salt of erdafitinib, in particular an L-lactate salt of erdafitinib and at least one pharmaceutical excipient. In some embodiments, the at least one pharmaceutical excipient comprises or is selected from a solubilizer, a binder, a diluent (filler), a wetting agent, a disintegrant, a glidant, a lubricant, a formaldehyde scavenger, an osmotic agent, or any combination thereof. In some embodiments, the at least one pharmaceutical excipient comprises or is selected from a binder, a diluent (filler), a glidant, a lubricant, or any combination thereof.

In some embodiments, the binder comprises hydroxypropyl methyl cellulose, hydroxypropyl cellulose, polyvinylpyrrolidone (PVP), vinylpyrrolidone-vinyl acetate (PVP-VA), or a combination thereof. In some embodiments, the binder is present in the drug formulation at a total concentration of between about 1 wt % and about 30 wt %, between about 5 wt % and about 20 wt %, or between about 10 wt % and about 15 wt %.

In some embodiments, the diluent (filler) comprises microcrystalline cellulose, silicified microcrystalline cellulose, dibasic calcium phosphate, or a combination thereof. In some embodiments, the diluent (filler) is present in the drug formulation at a total concentration of between about 5 wt % and about 30 wt %, between about 10 wt % and about 30 wt %, or between about 10 wt % and about 20 wt %.

In some embodiments, the glidant comprises hydrophilic colloidal silicon dioxide or hydrophobic colloidal silicon dioxide. In some embodiments, the glidant is present in the drug formulation at a total concentration of between about 0.05 wt % and about 1 wt %, between about 0.1 wt % and about 0.5 wt %, or about 0.25 wt %.

In some embodiments, the lubricant comprises magnesium stearate or sodium stearyl fumarate or polyethylene glycol. In some embodiments, the lubricant comprises magnesium stearate or sodium stearyl fumarate. In some embodiments, the lubricant comprises magnesium stearate. In some embodiments, the lubricant is present in the drug formulation at a total concentration of between about 0.05 wt % and about 5 wt %, between about 1 wt % and about 5 wt %, or about 2.5%.

In some embodiments, the pharmaceutical composition, or the drug delivery system comprising said pharmaceutical composition, comprises an intragranular composition or an intragranular fraction and an extragranular composition or an extragranular fraction. In some embodiments, the intragranular composition or fraction comprises erdafitinib or a pharmaceutically acceptable salt thereof, in particular a lactate salt of erdafitinib, e.g. erdafitinib L-lactate. In some embodiments, the intragranular composition or fraction comprises erdafitinib or a pharmaceutically acceptable salt thereof, in particular a lactate salt of erdafitinib, e.g. erdafitinib L-lactate, and at least one intragranular excipient, and the extragranular composition or the extragranular fraction comprises at least one extragranular excipient. In some embodiments, the at least one intragranular excipient and the at least one extragranular excipient do not comprise a common pharmaceutical excipient. In some embodiments, the at least one intragranular excipient comprises an intragranular binder. In some embodiments, the intragranular binder comprises hydroxypropyl methyl cellulose. In some embodiments, the at least one extragranular excipient comprises one or more of an extragranular binder, extragranular filler (diluent), extragranular glidant, and extragranular lubricant. In some embodiments, the extragranular binder comprises vinylpyrrolidone-vinyl acetate. In some embodiments, the extragranular diluent (filler) comprises microcrystalline cellulose, silicified microcrystalline cellulose, or a combination thereof. In some embodiments, the extragranular glidant comprises or is colloidal silicon dioxide (hydrophilic). In some embodiments, the extragranular lubricant comprises magnesium stearate or sodium stearyl fumarate or polyethylene glycol. In some embodiments, the extragranular lubricant comprises magnesium stearate or sodium stearyl fumarate. In some embodiments, the extragranular lubricant comprises or is magnesium stearate. In some embodiments, the at least one extragranular excipient comprises a filler and a binder, in particular microcrystalline cellulose and vinyl vinylpyrrolidone-vinyl acetate, in particular wherein the weight:weight ration of filler to binder is 1:1.

In some embodiments, the pharmaceutical composition, or the drug delivery system comprising said pharmaceutical composition, comprises the erdafitinib L-lactate salt in a concentration of from 60 wt % to 91 wt %. In some embodiments, the pharmaceutical composition, or the drug delivery system comprising said pharmaceutical composition, comprises the erdafitinib L-lactate salt in a concentration of from 60 wt % to 80 wt %. In some embodiments, the erdafitinib L-lactate salt is present in the drug formulation in a concentration of 70 wt %.

In some embodiments, the pharmaceutical composition, or the drug delivery system comprising said pharmaceutical composition, comprises the erdafitinib L-lactate salt in the form of a plurality of mini-tablets. In some embodiments, the pharmaceutical composition is in the form of between about 10 and about 100 mini-tablets. In some embodiments, the pharmaceutical composition comprises a total length of from about 14.5 cm to about 15 cm of mini-tablets, and/or (b) the formulation comprises from about 920 mg to about 965 mg of mini-tablets. In some embodiments, the pharmaceutical composition comprises a total length of from about 14.5 cm to about 15 cm of mini-tablets, and/or (b) the formulation comprises from about 920 mg to about 950 mg of mini-tablets.

In some embodiments, the pharmaceutical composition comprising erdafitinib L-lactate salt is in the form of a tablet that has a hardness of at least about 100 N. In some embodiments, the tablet has a hardness of between about 150 N and about 250 N. In some embodiments, the tablet has a hardness of between about 175 N and about 225 N. In some embodiments, the tablet has a thickness of between about 3.2 mm and about 3.6 mm. In some embodiments, the tablet is a mini-tablet which is in the form of a solid cylinder having a cylindrical axis, a cylindrical side face, circular end faces perpendicular to the cylindrical axis, a diameter across the circular end faces, and a length along the cylindrical side face. In some embodiments, the length of the mini-tablet exceeds the diameter of the mini-tablet to provide the mini-tablet with an aspect ratio (length:diameter) of greater than 1:1. In some embodiments, the mini-tablet has a diameter of from 1.0 mm to 3.2 mm, or from 1.5 mm to 3.1 mm. In some embodiments, the mini-tablet has a diameter of from 2.5 mm to 2.7 mm. In some embodiments, the mini-tablet has a length of 3.0 mm to 3.5 mm. In some embodiments, the mini-tablet has a mass of 22 mg to 24 mg.

In another aspect, provided is a process for making said pharmaceutical composition in the form of a tablet, comprising. (a) preparing an intragranular solid composition comprising erdafitinib L-lactate and at least one intragranular pharmaceutical excipient; (b) combining the intragranular solid composition with at least one extragranular pharmaceutical excipient to form a blend; and (c) tableting the blend to form the solid pharmaceutical composition. In some embodiments, (a) the at least one intragranular pharmaceutical excipient comprises at least one intragranular binder; and (b) the at least one extragranular pharmaceutical excipient comprises an extragranular binder, an extragranular filler (diluent), an extragranular glidant, and an extragranular lubricant. In some embodiments, the intragranular solid composition is prepared by a fluid bed granulation process. In some embodiments, the at least one intragranular binder comprises hydroxypropyl methyl cellulose. In some embodiments, the at least one extragranular binder comprises vinyl pyrrolidone-vinyl acetate (PVP VA). In some embodiments, the at least one extragranular filler (diluent) comprises microcrystalline cellulose. In some embodiments, the extragranular filler (diluent) further comprises a second extragranular filler (diluent), which comprises silicified microcrystalline cellulose. In some embodiments, the extragranular glidant comprises or is colloidal silicon dioxide (hydrophilic). In some embodiments, the extragranular lubricant comprises or is magnesium stearate. In some embodiments, the extragranular lubricant comprises sodium stearyl fumarate. In some embodiments, the extragranular lubricant comprises polyethylene glycol. In some embodiments, the at least one extragranular excipient comprises a filler and a binder, in particular microcrystalline cellulose and vinyl vinylpyrrolidone-vinyl acetate, in particular wherein the weight:weight ration of filler to binder is 1:1. In some embodiments, the ejection force is below about 1000 N.

### BRIEF DESCRIPTION OF THE DRAWINGS

The detailed description is set forth with reference to the accompanying drawings. The use of the same reference numerals may indicate similar or identical items. Various embodiments may utilize elements and/or components other than those illustrated in the drawings, and some elements and/or components may not be present in various embodiments. Elements and/or components in the figures are not necessarily drawn to scale.

FIG. 1A is a plan view of one embodiment of a drug delivery system in a coiled retention shape, in accordance with the present disclosure.

FIG. 1B is a cross-sectional view of one embodiment of the drug delivery system shown in FIG. 1A, taken along line B-B.

FIG. 2 is a schematic illustrating operation of one embodiment of an osmotic drug delivery system in accordance with present disclosure.

FIG. 3 is a perspective view of one embodiment of a mini-tablet form of a drug formulation in accordance with the present disclosure.

FIG. 4 shows single-dose erdafitinib exposures in plasma from nude rats bearing subcutaneous or orthotopic UM-UC-1 tumors. Exposure levels were measured in plasma from naïve, orthotopic bladder, or s.c. UM-UC-1 tumor-bearing nude rats. Rats were dosed with a single IVES (1-hour instillation) or p.o. dose of erdafitinib at the dose levels indicated. Individual data points are shown, with the mean represented by a horizontal line at each time point. IVES, intravesical, PO or p.o., oral; s.c., subcutaneous.

FIG. 5 demonstrates the effect of erdafitinib on ERK 1/2 phosphorylation in orthotopic bladder UM-UC-1 tumors. Individual pERK and total ERK levels were measured from UM-UC-1 orthotopic bladder tumors from nude rats treated with vehicle, or a single IVES (1-hour instillation) or p.o. dose of erdafitinib at the dose levels indicated. pERK and total ERK levels are reported as a ratio (pERK/ERK) relative to the mean of the vehicle group for the corresponding timepoint, with the exception of the 120-hour timepoint, where values were normalized to the 48-hour vehicle group. Individual data points are shown, with the mean represented by a horizontal line at each time point. N=2-6/group; ERK, extracellular signal-regulated kinase; IVES, intravesical; pERK, phosphorylated extracellular signal-regulated kinase; PO or p.o., oral.

FIG. 6 shows the size of orthotopic bladder UC tumor examples versus control bladder at 14 days post implantation. Formalin was used to fix tissue samples after necropsy. UC, urothelial carcinoma; NBTII, rat Nara Bladder Tumor No. 2 cells; T24, human bladder carcinoma cells.

FIG. 7 is a schematic of the perfusion experiment in athymic rats with UM-UC-1 implanted into the bladder wall.

FIG. 8 shows the percentage change in body weight of athymic, bladder-cannulated rats bearing orthotopic UM-UC-1 bladder tumors. Graph values are expressed as mean±SEM of 10-13 animals in each group. Concentrations cited in the figure legend are nominal target urine concentrations. Statistical analysis was carried out by Two-way ANOVA followed by Bonferroni multiple comparison test using Graph Pad Prism (Version 8.3.0). Statistically non-significant difference when percentage change in body weight of erdafitinib (0.5, 1.0, and 5.0 μg/mL) treatment groups were compared with percentage change in body weight of the vehicle control group. SEM, standard error of the mean.

FIG. 9 shows the mean percentage tumor weight reduction after accounting for bladder weight without tumor. Values (Group 1-4) are expressed as mean±SEM of 10-13 animals in each group. Statistical analysis was carried out by One-way ANOVA followed by Dunnett's multiple comparisons test using Graph Pad Prism (Version 8.3.0). Conc, concentration; SEM, standard error of the mean.

FIG. 10 displays the percentage change in body weight of athymic, bladder-cannulated nude rats bearing orthotopic RT-112 bladder tumor. Values are expressed as mean±SEM of 2-14 animals in each group. Concentrations cited in the figure legend are nominal target urine concentrations. Statistical analysis was carried out by Two-way ANOVA followed by Bonferroni multiple comparison test using Graph Pad Prism (Version 8.3.0). Statistically non-significant difference when percentage change in body weight of erdafitinib (0.5, 1.0, and 5.0 μg/mL) treatment groups were compared with percentage change in body weight of vehicle control group except Group 4 on Day 11 (*p<0.05). SEM, standard error of the mean.

FIG. 11 shows the effect of intravesical erdafitinib exposure on tumor growth as determined by changes in total bladder weight of athymic nude rats bearing orthotopic RT-112 bladder tumor. Values (Group 1-5) are expressed as mean±SEM of 2-14 animals in each group. Statistical analysis was carried out by One-way ANOVA followed by Dunnett's multiple comparisons test using Graph Pad Prism (Version 8.3.0). * p<0.05. Conc, concentration; SEM, standard error of the mean; ns, not significant.

FIGS. 12A and 12B show the plasma (FIG. 12A) and bladder (FIG. 12B) concentrations in rats following bladder perfusion of erdafitinib. Bladder perfusion of erdafitinib solution (0.1 mg/mL, 0.1 mL/hour, cumulative dose 0.72 mg) took place over 72 hours. Concentrations are expressed as average daily urine concentration in ng/mL.

FIG. 13 shows mean erdafitinib urine concentrations in pigs following bladder perfusion of erdafitinib at a constant rate of 12.5 mL/hour for 6 consecutive days for 2 animals and 8 consecutive days for 3 animals. All excreted urine was collected in 24-hour intervals through Day 6 or 8. Conc., concentration; SD, standard deviation.

FIG. 14 shows mean erdafitinib plasma concentrations in pigs following bladder perfusion of erdafitinib at a constant rate of 12.5 mL/hour for 6 consecutive days for 2 animals and 8 consecutive days for 3 animals. Blood samples were collected daily on study Days 1 to 8. SD, standard deviation.

FIG. 15 shows the screening results of materials permeation. O, permeable; Δ, practically impermeable; X, impermeable. ^{a}High variability between replicates.

FIGS. 16A-16C show schematics of the osmotic system design with the orifice+end plugs. FIG. 16A provides a cross-sectional side view of one embodiment of a device having a pre-formed sidewall orifice and two restraining end plugs. FIG. 16B provides a plan view of one embodiment of a device having a preformed sidewall orifice and two restraining end plugs. FIG. 16C provides a cross-sectional side view of an end portion of one embodiment of a device having restraining end plugs and microchannels formed through the end plugs.

FIG. 17 shows the IVR profile for Prototype 4, osmotic (orifice+end plugs), 0.2 mm wall, erdafitinib L-lactate, tablets (20 wt % water-insoluble excipients)). Erda, erdafitinib; IVR, in vitro release; SU, simulated urine; FBE, free base equivalents.

FIG. 18 shows the IVR profile for Prototype 5, osmotic (orifice+end plugs), 0.2 mm wall, erdafitinib L-lactate, tablets (water soluble excipients)). Erda, erdafitinib; IVR, in vitro release; SU, simulated urine; FBE, free base equivalents.

FIG. 19 summarizes the mean urine concentration versus time profiles in minipigs for Prototypes 4-5.

FIGS. 20A-20B shows the solubility of the erdafitinib L-lactate salt as a function of pH in water at 37° C. where the pH is adjusted using HCl/NaOH solutions (FIG. 20A) and the solubility of the erdafitinib L-lactate salt as a function of pH in simulated urine at 37° C. mg/mL (FIG. 20B).

FIG. 21 displays an overview of the synthesis reaction of the erdafitinib mono L-lactate salt (JNJ-42756493-AFK) from erdafitinib base (JNJ-42756493-AAA).

### DETAILED DESCRIPTION

In some embodiments, erdafitinib solid formulations are provided containing a high concentration of erdafitinib, which are designed for intravesical drug delivery and controlled and extended drug release when deployed within the bladder. In some embodiments, the solid erdafitinib formulations are further tailored for large scale manufacturing and to provide structural and chemical integrity of the solid formulations, in particular tablets, when used in an intravesical drug delivery system. Improved intravesical drug delivery systems and methods of drug delivery are also provided. In a particular embodiment, systems are configured for intravesical insertion and sustained drug delivery, preferably providing a zero order release rate of therapeutically effective amounts of the drug, in particular erdafitinib.

Described herein are erdafitinib formulations and release systems that are tailored for intravesical drug delivery, in order to take advantage of this route of administration. When formulated in solid form and administered in a suitable intravesical drug delivery system, such formulations might provide a controlled drug release rate and an extended drug release profile. Further provided are systems capable of delivering erdafitinib at effective release rates for the local treatment of bladder cancer.

In particular embodiments, the drug delivery system described herein is a drug device combination, consisting of a device constituent, particularly an intravesical device, and a drug constituent, particularly an erdafitinib formulation, such as erdafitinib tablets.

### The Erdafitinib Formulation & Tablets

In one aspect, erdafitinib lactate salt, in particular erdafitinib L-lactate, in particular erdafitinib mono L-lactate, is disclosed. It is useful, for example, in the local delivery/administration of erdafitinib to patients. In embodiments, a pharmaceutical composition is provided which comprises erdafitinib lactate salt, in particular erdafitinib L-lactate, and one or more excipients. In embodiments, the pharmaceutical composition is in the form of a tablet, particularly a mini-tablet.

In one aspect, this disclosure provides erdafitinib formulations, in particular erdafitinib tablets suitable for use in the disclosed intravesical drug delivery system. In particular, drug tablets comprising salt forms of erdafitinib (N-(3,5-dimethoxyphenyl)-N′-(1-methylethyl)-N-[3-(1-methyl-1H-pyrazol-4-yl)quinoxalin-6-yl]ethane-1,2-diamine) are provided. After the drug delivery system is inserted intravesically, the drug is released from the system into the bladder. In an aspect for example, the drug delivery system may operate as an osmotic pump, which produces a continuous release of the drug into the bladder over an extended period as the drug is released from the tablets in the system.

In order to increase or maximize the amount of drug that can be stored in and released from the disclosed drug delivery system, the drug tablets can have a relatively high erdafitinib content by weight. This relatively high weight fraction of erdafitinib in the drug tablet is attended by a reduced or low weight fraction of excipients which may be required for tablet manufacturing and system assembly and drug use considerations. For the purposes of this disclosure, terms such as “weight fraction,” “weight percentage,” and “percentage by weight” with reference to any drug or API (active pharmaceutical ingredient) refers to the drug or API in the form employed, whether in free base form, free acid form, salt form, or hydrate form. For example, a drug tablet that has 90% by weight (90 wt %) of a drug or excipient in salt form may include less than 90% by weight of that drug in free base form.

The erdafitinib drug tablet of this disclosure includes an erdafitinib content and an excipient content. The drug content can include one form or more than one form of erdafitinib, such as free base or salt form, and the excipient content can include one or more excipients. Particular embodiments include salts of erdafitinib, and more particularly erdafitinib lactate salts, such as a mono L-lactate of erdafitinib. The term “excipient” is known in the art, and representative examples of excipients useful in the disclosed drug tablets may include but are not limited to ingredients such as binders, lubricants, glidants, disintegrants, solubilizers, colors, fillers or diluents, wetting agents, stabilizers, formaldehyde scavengers, coatings, and preservatives, or any combination thereof, as well as other ingredients to facilitate manufacturing, storing, or administering the drug tablet.

In embodiments, the erdafitinib drug tablets include erdafitinib in a salt form. In one aspect, erdafitinib drug tablets can include greater than or equal to 50 wt % erdafitinib L-lactate, with the remainder of the weight comprising excipients, such as lubricants, binders, and stabilizers that facilitate making and using the drug tablet. In embodiments, the erdafitinib drug tablets include erdafitinib in a salt form. In one aspect, erdafitinib drug tablets can include greater than or equal to 50 wt % erdafitinib L-lactate, with the remainder of the weight comprising excipients, such as lubricants, fillers, binders, and glidants that facilitate making and using the drug tablet. Alternatively, the erdafitinib drug tablets can include greater than or equal to 40 wt %, greater than or equal to 45 wt %, greater than or equal to 50 wt %, greater than or equal to 55 wt %, greater than or equal to 60 wt %, greater than or equal to 65 wt %, greater than or equal to 70 wt %, or greater than or equal to 75 wt %, greater than or equal to 80 wt %, greater than or equal to 85 wt %, or greater than or equal to 90 wt %, erdafitinib L-lactate. In an aspect, the drug tablets can include from 50 wt % to 90 wt %, from 65 wt % to 85 wt %, from 70 wt % to 80 wt %, such as 70 wt %, or 75 wt %, or 80 wt %, of erdafitinib in its L-lactate form, based on the total weight of the tablet.

In embodiments, the erdafitinib in the erdafitinib drug tablets is in the form of the L-lactate salt of erdafitinib. Properties of erdafitinib L-lactate which make it advantageous for use in the disclosed osmotic system include its high solubility, its stability, and its ability to function as an osmotic agent without the requirement of an added osmotic agent.

Erdafitinib formulations with a range of excipient combinations, both intragranular and extragranular, are provided in Table 2, Table 9, Table 13, and Table 16, in the Examples. In particular, Table 16 sets forth formulation concepts 1-5. Concepts 1-5 are formulation embodiments encompassed by this disclosure.

In some embodiments, the solid pharmaceutical composition comprises: (a) 70 wt % erdafitinib L-lactate; (b) 1.43 wt % hydroxypropyl methyl cellulose; (c) 9.30 wt % microcrystalline cellulose; (d) 7.22 wt % silicified microcrystalline cellulose; (e) 9.30 wt % vinyl pyrrolidone-vinyl acetate (PVP VA); (f) 0.25 wt % colloidal silicon dioxide (hydrophilic); and (g) 2.5 wt % magnesium stearate, wherein the weight percentages are relative to the entire solid pharmaceutical composition. In some embodiments, the solid pharmaceutical composition comprises (a) 70 wt % erdafitinib L-lactate, intragranular; (b) 1.43 wt % hydroxypropyl methyl cellulose, intragranular; (c) 9.30 wt % microcrystalline cellulose, extragranular; (d) 7.22 wt % silicified microcrystalline cellulose, extragranular; (e) 9.30 wt % vinyl pyrrolidone-vinyl acetate (PVP VA), extragranular; (f) 0.25 wt % colloidal silicon dioxide (hydrophilic), extragranular; and (g) 2.5 wt % magnesium stearate, extragranular. In some embodiments, the solid pharmaceutical composition is a tablet. In some embodiments, the formulation is Concept 1

In some embodiments, the solid pharmaceutical composition comprises: (a) 70 wt % erdafitinib L-lactate; (b) 1.43 wt % hydroxypropyl methyl cellulose; (c) 12.91 wt % microcrystalline cellulose; (d) 12.91 wt % vinyl pyrrolidone-vinyl acetate (PVP VA); (c) 0.25 wt % colloidal silicon dioxide (hydrophilic); and (f) 2.5 wt % magnesium stearate, wherein the weight percentages are relative to the entire solid pharmaceutical composition. In some embodiments, the solid pharmaceutical composition comprises: (a) 70 wt % erdafitinib L-lactate, intragranular; (b) 1.43 wt % hydroxypropyl methyl cellulose, intragranular; (c) 12.91 wt % microcrystalline cellulose, extragranular; (d) 12.91 wt % vinyl pyrrolidone-vinyl acetate (PVP VA), extragranular; (e) 0.25 wt % colloidal silicon dioxide (hydrophilic), extragranular; and (f) 2.5 wt % magnesium stearate, extragranular. In some embodiments, the solid pharmaceutical composition is a tablet. In some embodiments, the formulation is Concept 2.

In some embodiments, the solid pharmaceutical composition comprises: (a) 70 wt % erdafitinib L-lactate; (b) 1.43 wt % hydroxypropyl methyl cellulose; (c) 12.91 wt % microcrystalline cellulose; (d) 12.91 wt % vinyl pyrrolidone-vinyl acetate (PVP VA); (e) 0.25 wt % colloidal silicon dioxide (hydrophilic); and (f) 2.5 wt % sodium stearyl fumarate, wherein the weight percentages are relative to the entire solid pharmaceutical composition. In some embodiments, the solid pharmaceutical composition comprises: (a) 70 wt % erdafitinib L-lactate, intragranular; (b) 1.43 wt % hydroxypropyl methyl cellulose, intragranular; (c) 12.91 wt % microcrystalline cellulose, extragranular; (d) 12.91 wt % vinyl pyrrolidone-vinyl acetate (PVP VA), intragranular; (e) 0.25 wt % colloidal silicon dioxide (hydrophilic), extragranular; and (f) 2.5 wt % sodium stearyl fumarate, extragranular. In some embodiments, the solid pharmaceutical composition is a tablet. In some embodiments, the formulation is Concept 3.

In some embodiments, the solid pharmaceutical composition comprises: (a) 60 wt % erdafitinib L-lactate; (b) 1.23 wt % hydroxypropyl methyl cellulose; (c) 18.01 wt % microcrystalline cellulose; (d) 18.01 wt % vinyl pyrrolidone-vinyl acetate (PVP VA); (e) 0.25 wt % colloidal silicon dioxide (hydrophilic); and (f) 2.5 wt % magnesium stearate, wherein the weight percentages are relative to the entire solid pharmaceutical composition. In some embodiments, the solid pharmaceutical composition comprises: (a) 60 wt % erdafitinib L-lactate, intragranular; (b) 1.23 wt % hydroxypropyl methyl cellulose, intragranular; (c) 18.01 wt % microcrystalline cellulose, extragranular; (d) 18.01 wt % vinyl pyrrolidone-vinyl acetate (PVP VA), extragranular; (e) 0.25 wt % colloidal silicon dioxide (hydrophilic), extragranular; (f) 2.5 wt % magnesium stearate, extragranular. In some embodiments, the solid pharmaceutical composition is a tablet. In some embodiments, the formulation is Concept 4.

In some embodiments, the solid pharmaceutical composition comprises: (a) 80 wt % erdafitinib L-lactate; (b) 1.63 wt % hydroxypropyl methyl cellulose; (c) 7.81 wt % microcrystalline cellulose; (d) 7.81 wt % vinyl pyrrolidone-vinyl acetate (PVP VA); (e) 0.25 wt % colloidal silicon dioxide (hydrophilic); and (f) 2.5 wt % magnesium stearate, wherein the weight percentages are relative to the entire solid pharmaceutical composition. In some embodiments, the solid pharmaceutical composition comprises: (a) 80 wt % erdafitinib L-lactate, intragranular; (b) 1.63 wt % hydroxypropyl methyl cellulose, intragranular; (c) 7.81 wt % microcrystalline cellulose, extragranular; (d) 7.81 wt % vinyl pyrrolidone-vinyl acetate (PVP VA), extragranular; (e) 0.25 wt % colloidal silicon dioxide (hydrophilic), extragranular; and (f) 2.5 wt % magnesium stearate, extragranular. In some embodiments, the solid pharmaceutical composition is a tablet. In some embodiments, the formulation is Concept 5.

In embodiments, the erdafitinib drug and excipients are selected and the tablet is formulated to permit release of the drug from the tablet. In embodiments, the erdafitinib is formulated in a pharmaceutical composition to be sterilizable, either within or outside of the drug delivery system, without resulting in substantial or detrimental changes to the chemical or physical composition of the drug tablets which would otherwise make them unsuitable for delivering the erdafitinib as described herein. In an aspect, the erdafitinib drug and excipients are selected for their suitability for sterilization processes. In an embodiment, the drug delivery system comprising the drug tablets is sterilized as a whole. In particular, the drug delivery system comprising the drug tablets is sterilized by gamma irradiation.

In an aspect, the erdafitinib drug tablets may be sized and shaped for use with an indwelling drug delivery system including the intravesical drug delivery system disclosed herein. For example, the erdafitinib drug tablets may be “mini-tablets” that are generally smaller in size than conventional tablets, which may permit inserting the system-housed drug tablets through a natural body lumen such as the urethra into a cavity such as the bladder. The erdafitinib tablets may be coated or uncoated. In particular, uncoated tablets may work well in combination with the disclosed delivery system.

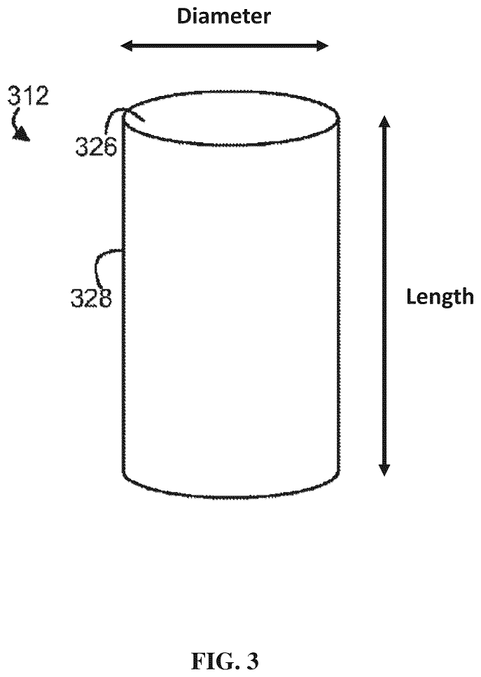

An example of a mini-tablet is shown in FIG. 3, which illustrates a mini-tablet 312 having circular flat end faces 326 and a cylindrical side wall 328.

In embodiments, the drug tablet for intravesical insertion can be in the form of a solid cylinder having a cylindrical axis, a cylindrical side face, circular end faces perpendicular to the cylindrical axis, a diameter across the circular end faces, and a length along the cylindrical side face. In cylindrical form, each mini-tablet can have a length (L) exceeding its diameter (D) so that the mini-tablet has an aspect ratio (L:D) of greater than 1:1. For example, the aspect ratio (L:D) of each mini-tablet can be 1.6, 1.5, 1.4, 1.3, 1.2, 1.1, or range in values between these aspect ratios. Embodiments of the mini-tablet can have a cylindrical diameter of from 1.0 mm to 3.2 mm, or from 1.5 mm to 3.1 mm, or from 2.0 mm to 2.7 mm, or from 2.5 mm to 2.7 mm. In some aspects, the mini-tablet can have a diameter of from 2.4 mm to 2.8 mm. In some aspects, the mini-tablet can have a length of from 1.7 mm to 4.8 mm, or from 2.0 mm to 4.5 mm, or from 2.8 mm to 4 mm, or from 3 mm to 3.5 mm. In some aspects, the mini-tablet can have a length from 3.0 mm to 3.4 mm.

The API used in the solid tablet formulations can be erdafitinib, which is N-(3,5-dimethoxyphenyl)-N′-(1-methylethyl)-N-[3-(1-methyl-1H-pyrazol-4-yl)quinoxalin-6-yl]ethane-1,2-diamine, and the chemical structure of which is illustrated below. Erdafitinib tablets for use in the disclosed intravesical system can be formulated using a salt of the erdatitinib. In some particular embodiments, the erdafitinib tablets for use in the disclosed intravesical system can include a lactate salt of erdafitinib. In a particular embodiment, the erdafitinib tablets for use in the disclosed intravesical system can be the L-lactate salt of erdafitinib, in particular the mono L-lactate salt of erdafitinib.

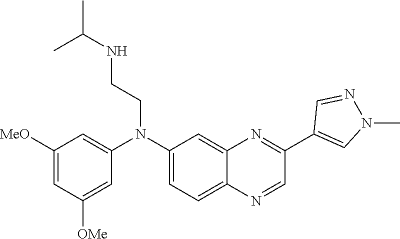 [SMILES: COc1cc(OC)cc(N(CCNC(C)C)c2ccc3ncc(-c4cnn(C)c4)nc3c2)c1]

The general reference to a compound includes all stereoisomers unless explicitly indicated otherwise, and the reference to a general structure or name encompasses all enantiomers, diastereomers, and other optical isomers whether in enantiomeric or racemic forms, as well as mixtures of stereoisomers, as the context permits or requires. For any particular formula or name that is presented, any general formula or name presented also encompasses all stereoisomers that can arise from a particular set of substituents. Accordingly, for example, as used herein, reference to “a lactate salt” encompasses both D- and L-isomers.

In embodiments, the erdafitinib drug tablet can incorporate various excipients which include, but are not limited to, at least one solubilizer, at least one binder, at least one wetting agent, at least one disintegrant, at least one stabilizer, at least one diluent, at least one glidant, at least one lubricant, at least one osmotic agent (osmogen), and the like, or any combination thereof. In embodiments, the erdafitinib drug tablet can be absent various excipients or combinations of excipients, including but not limited to, a solubilizer, a wetting agent, at least one stabilizer, at least one disintegrant, at least one osmotic agent (osmogen), or combinations thereof. In embodiments, the erdafitinib drug tablet comprises at least one binder, at least one filler (diluent), at least one glidant, at least one lubricant, or any combination thereof. Any excipient or any combination of the excipients can be present in the intragranular composition, the extragranular composition, or both the intragranular and the extragranular composition. In an aspect, at least one intragranular pharmaceutical excipient and at least one extragranular pharmaceutical excipient can be the same, that is, can be selected from at least one common (mutually occurring) pharmaceutical excipient. In a further aspect, the intragranular pharmaceutical excipients and the extragranular pharmaceutical excipients do not comprise a common (mutually occurring) pharmaceutical excipient, such that the intragranular and the extragranular excipients are mutually exclusive. In embodiments, the erdafitinib drug tablets, in particular the erdafitinib lactate drug tablets comprising from 50 wt % to 90 wt %, from 60 wt % to 80 wt %, from 65 wt % to 75 wt %, such as 70 wt %, of erdafitinib in its lactate form, in particular its L-lactate form, includes at least one binder, at least one diluent, at least one glidant, at least one lubricant, or any combination thereof.

It will be appreciated that these functional descriptions of various excipients are used generally as follows. A solubilizer can improve or enhance the solubility of the API within the drug lumen of the disclosed system following in vivo insertion. A binder can hold the solid particles of the composition together for physical stability. A wetting agent can lower the surface tension between the drug and the medium in which it occurs and help maintain the solubility of the drug. A disintegrant can aid in the mini-tablet disintegration when contacting water (urine) to release the drug substance. A stabilizer can improve the chemical stability such as the thermal stability of the formulation, including the API, or protects the API against degradation. A diluent can function as a bulking agent to increase the volume or weight of the composition which may aid in providing tablet of the desired size. A glidant may improve the flow properties of the (granulated) particles of tablet components or of the powder blend to be tableted. A lubricant can prevent particles of the composition from adhering to components of the manufacturing apparatus, such as dies and punches of a tablet press. An osmotic agent dissolves in an aqueous fluid in the delivery system and creates an osmotic pressure build up therein.

In embodiments, the erdafitinib L-lactate formulation can include all or only some of these excipients. For example, in an aspect, erdafitinib L-lactate formulation can be absent a solubilizer, absent a wetting agent, absent a disintegrant, absent a stabilizer (for example, absent meglumine), absent an osmotic agent, or absent any combination of these excipients. For example, in another aspect, the drug itself, such as the lactate salt of erdafitinib, can function as an osmotic agent. In an aspect, the drug itself, such as the lactate salt of erdafitinib, functions as an osmotic agent and the erdafitinib lactate formulation does not comprise another osmotic agent. In an aspect, an excipient can be water soluble. In another aspect, an excipient can be colloidal in water. According to another aspect, an excipient can be soluble under the conditions of its deployment in the patient, such as in a bladder. These and other excipients are described in more detail below.

### Stabilizers Such as Formaldehyde Scavengers

In an aspect, erdafitinib API may be sensitive to degradation under certain conditions when incorporated into a solid formulation. For example, erdafitinib can degrade or transform in the presence of formaldehyde, to form the cyclization product 6,8-dimethoxy-4-(1-methylethyl)-1-[3-(1-methyl-1H-pyrazol-4-yl)quinoxalin-6-yl]-2,3,4,5-tetrahydro-1H-1,4-benzodiazepine. Formaldehyde can come into contact with the erdafitinib from a variety of sources in the environment, such as from packaging materials or as a contaminant in excipients or other components of the formulation.

Accordingly, in one aspect, the erdafitinib pharmaceutical formulation can include a formaldehyde scavenger to improve the stability or shelf life of the formulation. Various formaldehyde scavengers can be employed which can prevent, slow down, diminish, or postpone the formation of degradation products when erdafitinib contacts formaldehyde. Therefore, the erdafitinib pharmaceutical formulation stability such as its chemical stability can be increased in the presence of a formaldehyde scavenger as compared to an erdafitinib pharmaceutical formulations absent a formaldehyde scavenger. In an aspect, the formaldehyde scavenger can be present in the solid pharmaceutical composition as a component of the intragranular solid composition, the extragranular solid composition, or both the intragranular and extragranular composition. In an aspect, the formaldehyde scavenger, in particular meglumine, may be present in the solid pharmaceutical composition as a component of the intragranular solid composition.

Formaldehyde scavengers can include or can be selected from compounds comprising a reactive nitrogen center, such as compounds containing amine or amide groups. Without being bound by theory, it is thought that these compounds can react with formaldehyde to form a Schiff base imine (R¹R²C═NR³, where R³ is not hydrogen), which itself can bind formaldehyde. Examples of such formaldehyde scavengers include but are not limited to amino acids, amino sugars, alpha-(α-)amine compounds, conjugates and derivatives thereof, and mixtures thereof. Such formaldehyde scavenger compounds can include two or more amine and/or amide moieties which can scavenge formaldehyde.

In an aspect, formaldehyde scavengers can include or can be selected from, for example, meglumine, glycine, alanine, serine, threonine, cysteine, valine, leucine, isoleucine, methionine, phenylalanine, tyrosine, aspartic acid, glutamic acid, arginine, lysine, ornithine, taurine, histidine, aspartame, proline, tryptophan, citrulline, pyrrolysine, asparagine, glutamine, tris(hydroxymethyl)aminomethane, conjugates thereof, pharmaceutically acceptable salts thereof, or any combination thereof. According to an aspect, the formaldehyde scavenger can include or can be selected from meglumine or a pharmaceutically acceptable salt thereof, in particular meglumine base.

Therefore, an aspect of this disclosure is the use of a formaldehyde scavenger, in particular meglumine, in an erdafitinib pharmaceutical formulation such as a drug tablet formulation, to increase the stability of erdafitinib in any of its forms, including erdafitinib salt forms such as erdafitinib L-lactate. The chemical stability of the erdafitinib pharmaceutical formulation is increased as compared to an erdafitinib pharmaceutical formulation or composition containing no formaldehyde scavenger. An aspect of the disclosure is a method of preventing, slowing down, diminishing, or postponing the formation of degradation products such as the following compound, which can form from erdafitinib in the presence of formaldehyde:

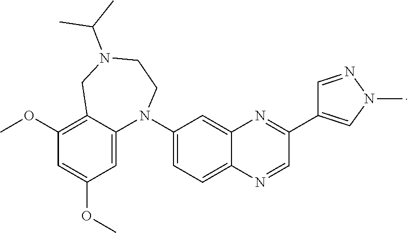

In an aspect, degradation products such as the above can occur in a solid tablet composition such as a mini-tablet formulation, in particular in a mini-tablet as disclosed herein.

When present in the erdafitinib solid pharmaceutical composition, the formaldehyde scavenger may be present in the solid pharmaceutical composition in a concentration of from 0.01 wt % to 5 wt %, from 0.05 wt % to 3 wt %, from 0.1 wt % to 2 wt %, from 0.5 wt % to 1.5 wt %, or about 1 wt %. When present in the erdafitinib solid pharmaceutical composition, the formaldehyde scavenger can be present in the solid pharmaceutical composition in a concentration of from 5 wt % to 10 wt %, about 5 wt %, about 6 wt %, about 7 wt %, about 8 wt %, about 9 wt % or about 10 wt %.

In an aspect, the pharmaceutical compositions as described herein, in particular the erdafitinib drug tablets, do not contain a stabilizer or formaldehyde scavenger.

### Solubilizers

In an aspect, the erdafitinib formulation can include a solubilizer. The solubilizer can be in the intragranular component, the extragranular component, or both the intragranular and extragranular component of the formulation. In embodiments, the solubilizer can comprise or can be selected from, for example (a) a cyclic oligosaccharide, (b) a cellulose which is functionalized with methoxy-, 2-hydroxypropoxy-, acetyl-, or succinoyl-moieties or a combination thereof, or (c) a salt thereof. In an embodiment, the solubilizer is present in the intragranular component. In other aspects, the erdafitinib formulation can be absent a solubilizer.

In embodiments, solubilizers for the erdafitinib tablet formulation can comprise or can be selected from an oligosaccharide. In embodiments, the solubilizer can comprise or can be selected from a cyclic oligosaccharide such as a cyclodextrin. Suitable cyclodextrin solubilizers for the erdafitinib tablet formulation include, but are not limited to, hydroxypropyl-beta-cyclodextrin, hydroxypropyl-gamma-cyclodextrin, sulfobutyl ether-beta-cyclodextrin sodium salt, or any combination thereof. In other embodiments, the solubilizer can comprise or can be hydroxypropyl methylcellulose E5 (HPMC-E5). In other embodiments, solubilizers for the erdafitinib tablet formulation can comprise or can be hydroxypropyl methylcellulose acetate succinate.

Oligosaccharide solubilizers can be present in erdafitinib tablet form in a concentration of from 1 wt % to 20 wt %, alternatively from 3 wt % to 18 wt %, alternatively from 5 wt % to 15 wt %, alternatively from 7 wt % to 12 wt %, or alternatively 10 wt % or about 10 wt %. The cyclodextrin solubilizer can be present in an erdafitinib tablet formulation, for example an erdafitinib salt formulation, in a concentration of 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, or 20 wt %, or any range between any of these weight percentages.

In an aspect, a solubilizer for the erdafitinib tablet formulation disclosed herein can comprise or can be hydroxypropyl-beta-cyclodextrin. One embodiment of an erdafitinib formulation includes a hydroxypropyl-beta-cyclodextrin solubilizer, which may be present in the intragranular composition. In an aspect, the pharmaceutical compositions as described herein, in particular the erdafitinib drug tablets, do not contain a solubilizer.

### Binders

Pharmaceutical excipients for the erdafitinib solid pharmaceutical composition may include one or more binders. The one or more binders can be present in the solid pharmaceutical composition as a component of the intragranular solid composition, the extragranular solid composition, or both the intragranular and extragranular solid compositions. Suitable binders can be water soluble, water insoluble, or slightly water soluble or combinations of these. In certain embodiments, the binders are an aspect, binders can include water soluble binders such as water soluble polymeric binders. Polymeric binders can include non-ionic polymers which are pH stable in aqueous solution.

It will be appreciated by the person of ordinary skill that binders may also function as a diluent (also termed filler) in a pharmaceutical composition. Accordingly, binders provided in this disclosure may also be used for their diluent function as appropriate and unless otherwise indicated.

In an aspect, suitable binders can include or can be selected from (but are not limited to) polyvinylpyrrolidone (PVP, also termed polyvidone, povidone, or poly(1-vinyl-2-pyrrolidinone)), poly(vinyl acetate) (PVA), vinylpyrrolidone-vinyl acetate copolymer, polyethylene oxide (PEO, also termed poly(ethylene glycol) or PEG), polypropylene oxide (PPO, also termed poly(propylene glycol) or PPG), an ethylene glycol-propylene glycol copolymer, a poloxamer, hydroxypropyl cellulose (HPC), hydroxypropyl methylcellulose (HPMC), microcrystalline cellulose, silicified microcrystalline cellulose, or combinations thereof. In an aspect, suitable binders can include or can be selected from polyvinylpyrrolidone (PVP, also termed polyvidone, povidone, or poly(1-vinyl-2-pyrrolidinone)), poly(vinyl acetate) (PVA), vinylpyrrolidone-vinyl acetate copolymer, polyethylene oxide (PEO, also termed poly(ethylene glycol) or PEG), polypropylene oxide (PPO, also termed poly(propylene glycol) or PPG), an ethylene glycol-propylene glycol copolymer, a poloxamer, hydroxypropyl cellulose (HPC), hydroxypropyl methylcellulose (HPMC), microcrystalline cellulose, or combinations thereof. In an aspect, suitable binders can include or can be selected from hydroxypropyl methylcellulose (HPMC), microcrystalline cellulose, vinylpyrrolidone-vinyl acetate copolymer, or combinations thereof. In an aspect, suitable binders can include or can be selected from hydroxypropyl methylcellulose (HPMC), vinylpyrrolidone-vinyl acetate copolymer (copovidone), or combinations thereof.

In further aspects, suitable binders can include or can be selected from polymers of or copolymers of vinylpyrrolidone (VP, also 1-vinyl-2-pyrrolidinone) and vinyl acetate (VA). Suitable binders also may include or may be selected from polymers of or copolymers of ethylene oxide (EO) and propylene oxide (PO). Again, these binders can be used in combinations with other binders such as in combination with microcrystalline cellulose, hydroxypropyl cellulose (HPC), or hydroxypropyl methylcellulose (HPMC).

In an aspect, the total concentration of the at least one binder in the solid pharmaceutical composition can be from 5 wt % to 30 wt %, from 10 wt % to 25 wt %, from 12 wt % to 22 wt %, or from 14 wt % to 19 wt %. In one aspect, the total concentration of the at least one binder in the solid pharmaceutical composition can be from about 1 wt % to about 30 wt %, from about 5 wt % to about 20 wt %, or from about 10 wt % to about 15 wt %.

According to another aspect, suitable polymeric binders can include or can be selected from a copolymer of vinylpyrrolidone and vinyl acetate, which can be termed poly(vinyl-pyrrolidone-co-vinyl acetate) or poly(VP-co-VA). Examples of suitable binders include Kollidon® VA64 and Kollidon® VA64 Fine (BASF, Ludwigshafen am Rhein, Germany), having a molecular weight (Mw) range of from 45,000 g/mol to 70,000 g/mol based on measuring the light scatter of a solution. Another suitable binder is Kollidon® K30.

In embodiments, the polymeric binders such as the vinylpyrrolidone-vinyl acetate copolymer can be present in the disclosed erdafitinib tablet formulation in a concentration of from 2 wt % to 15 wt %, alternatively from 4 wt % to 12 wt %, alternatively from 6 wt % to 10 wt %, or alternatively, 8 wt % or about 8 wt %. For example, the vinylpyrrolidone-vinyl acetate copolymer binder can be present in erdafitinib tablet formulation in a concentration of 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt % or any range between any of these weight percentages. In an aspect, the vinylpyrrolidone-vinyl acetate copolymer is present in the intragranular composition. In an aspect, the vinylpyrrolidone-vinyl acetate copolymer is present in the intragranular composition and said intragranular composition is prepared by roller compaction. In an aspect, the vinylpyrrolidone-vinyl acetate copolymer is present in the extragranular composition. In an aspect, the vinylpyrrolidone-vinyl acetate copolymer is present in the extragranular composition in a concentration of from 8 wt % to 14 wt %, or alternatively from 7.5 wt % to 18.5 wt %. In an aspect, the vinylpyrrolidone-vinyl acetate copolymer is present in the extragranular composition in a concentration of from 8 wt % to 14 wt %, or alternatively from 9 wt % to 13 wt %.

In an aspect, the binder can comprise or can be microcrystalline cellulose. For example, the microcrystalline cellulose can be present in the solid pharmaceutical composition in a concentration of from 5 wt % to 20 wt %, from 6 wt % to 15 wt %, or from 7 wt % to 12 wt %.

According to another aspect, the binder can comprise or can be silicified microcrystalline cellulose. For example, the silicified microcrystalline cellulose can be present in the solid pharmaceutical composition in a concentration of from 3 wt % to 18 wt %, from 4 wt % to 15 wt %, or from 5 wt % to 12 wt %.

According to another aspect, the binder can comprise or can be hydroxypropyl methylcellulose. For example, the hydroxypropyl methylcellulose, e.g. HPMC 290 15 mPa·s can be present in the solid pharmaceutical composition in a concentration of from 0.5 wt % to 5 wt %, or from 0.5 wt % to 2 wt %, or from 1 wt % to 2 wt %.

### Wetting Agents

Pharmaceutical excipients for the erdafitinib solid pharmaceutical composition may include one or more wetting agents. The one or more wetting agents can be present in the solid pharmaceutical composition in the intragranular solid composition, the extragranular solid composition, or both the intragranular and extragranular solid composition. In exemplary embodiments, the wetting agent can comprise or can be selected independently from an anionic surfactant or a non-ionic surfactant, in particular an anionic surfactant. For example, the wetting agent can comprise or can be selected independently from sodium lauryl sulfate, sodium stearyl fumarate, polysorbate 80, docusate sodium, or any combination thereof. In embodiments, the total concentration of the wetting agent in the solid pharmaceutical composition can be from 0.01 wt % to 2.5 wt %, from 0.05 wt % to 1.0 wt %, or from 0.1 wt % to 0.5 wt %. In an embodiment, the wetting agent is present in the intragranular composition.

In an embodiment, the erdafitinib solid pharmaceutical composition does not include one or more wetting agents.

### Disintegrants

Pharmaceutical excipients for the erdafitinib solid pharmaceutical composition may include one or more disintegrants. The one or more disintegrants can be present in the solid pharmaceutical composition in the intragranular solid composition, the extragranular solid composition, or both the intragranular and extragranular solid composition. In an embodiment, the disintegrant is present in the intragranular composition. In an embodiment, the disintegrant is present in the intragranular composition and said intragranular composition is prepared by roller compaction.

In exemplary embodiments, the disintegrant can comprise or can be selected independently from a functionalized polysaccharide or a crosslinked polymer. For example, in an aspect, the disintegrant can comprise or can be selected from, for example (a) a cellulose which is functionalized with methoxy-, 2-hydroxypropoxy-, or carboxymethoxy-moieties, a salt thereof, or a combination thereof, (b) a carboxymethylated starch, or (c) a crosslinked polymer.

In embodiments, the disintegrant can comprise or can be selected independently from hydroxypropyl methylcellulose, low-substituted hydroxypropylcellulose, crospovidone (crosslinked polyvinylpyrrolidone), croscarmellose sodium (cross-linked sodium carboxymethylcellulose), sodium starch glycolate, or any combination thereof.

When present, the disintegrant can be present in a range of concentrations. In embodiments, the total concentration of the disintegrant in the solid pharmaceutical composition can be from 0.1 wt % to 10 wt %, from 0.2 wt % to 8 wt %, from 0.5 wt % to 7 wt %, from 1 wt % to 5 wt %, or from 2 wt % to 3 wt %.

In an embodiment, the erdafitinib solid pharmaceutical composition does not include one or more disintegrants.

### Diluents or Fillers

Pharmaceutical excipients for the erdafitinib solid pharmaceutical composition may include one or more diluents. The one or more diluents can be present in the solid pharmaceutical composition as a component of the intragranular solid composition, the extragranular solid composition, or both the intragranular and extragranular solid composition.

In exemplary embodiments, diluents can comprise or can be selected from a sugar, starch, microcrystalline cellulose, a sugar alcohol, a hydrogen phosphate salt, a dihydrogen phosphate salt, a carbonate salt, or combinations thereof. In an aspect, diluents can comprise or can be selected from lactose, dextrin, mannitol, sorbitol, starch, microcrystalline cellulose, silicified microcrystalline cellulose, dibasic calcium phosphate, anhydrous dibasic calcium phosphate, calcium carbonate, sucrose, or any combination thereof. In an aspect, diluents can comprise or can be selected from microcrystalline cellulose, silicified microcrystalline cellulose, dibasic calcium phosphate, anhydrous dibasic calcium phosphate, or any combination thereof. In an aspect, diluents can comprise or can be selected from microcrystalline cellulose and silicified microcrystalline cellulose, or any combination thereof.

In embodiments, the total concentration of the diluent in the solid pharmaceutical composition can be from 10 wt % to 30 wt %, from 12 wt % to 30 wt %, from 15 wt % to 25 wt %, or from 18 wt % to 22 wt %, or from 10 wt % to 25 wt %, or from 10 wt % to 20 wt % or from 10 wt % to 15 wt %. In embodiments, the total concentration of the diluent in the solid pharmaceutical composition can be from 5 wt % to 30 wt %, from 10 wt % to 30 wt %, or from 10 wt % to 20 wt % In some embodiments, the diluent comprises microcrystalline cellulose, silicified microcrystalline cellulose, or a combination thereof. For example, in some aspects, the diluent can comprise or can be selected from microcrystalline cellulose in a concentration of from 15 wt % to 25 wt % or from 20 wt % to 22 wt %. For example, in some aspects, the diluent can comprise or can be selected from microcrystalline cellulose in a concentration of from 5 wt % to 20 wt % or from 7.5 wt % to 18.5 wt % or from 5 wt % to 15 wt % or from 8 wt % to 14 wt %, and/or silicified microcrystalline cellulose in a concentration of from 5 wt % to 10 wt % or of from 7 wt % to 8 wt %. In some embodiments, the diluent does not comprise silicified microcrystalline cellulose. In a further aspect, the diluent can comprise or can be selected from anhydrous dibasic calcium phosphate in a concentration of from 18 wt % to 20 wt %.

It will be appreciated by the person of ordinary skill that some of the diluents/fillers disclosed herein may also function as binders in the pharmaceutical composition. Accordingly, some compounds or materials may be described herein as providing a binder function and providing a diluent function.

### Glidants

Pharmaceutical excipients for the erdafitinib solid pharmaceutical composition may include one or more glidants. The one or more glidants can be present in the solid pharmaceutical composition as a component of the intragranular solid composition, the extragranular solid composition, or both the intragranular and extragranular solid composition. In an aspect, the glidant is present in the extragranular composition. As used in this disclosure, a glidant refers to a pharmaceutical excipient which improves or optimizes the particle flow properties of the granulated or powdered tablet components in particle form by decreasing the interaction, attraction, cohesion, or friction between particles.

In an aspect, glidants can include or can be selected from colloidal silicon dioxide, colloidal anhydrous silicon dioxide, talc, or any combination thereof. In embodiments, the total concentration of the glidant in the solid pharmaceutical composition can be from 0.01 wt % to 5 wt %, 0.05 wt % to 3 wt %, 0.1 wt % to 1 wt %, or about 0.2 wt %, or about 0.25 wt %, or about 0.3 wt %, about 0.35 wt %, or about 0.4 wt %, or about 0.45 wt % or about 0.5 wt %. In embodiments, the total concentration of the glidant in the solid pharmaceutical composition can be from about 0.05 wt % to about 1 wt %, about 0.1 wt % to about 0.5 wt %, or about 0.25 wt %. In an embodiment, the glidant is colloidal silicon dioxide. In some embodiments, the glidant is colloidal silicon dioxide (hydrophilic) In some embodiments, the glidant is colloidal silicon dioxide (hydrophobic).

### Lubricants

Pharmaceutical excipients for the erdafitinib solid pharmaceutical composition may include one or more lubricants. The one or more lubricants can be present in the solid pharmaceutical composition as a component of the intragranular solid composition, the extragranular solid composition, or both the intragranular and extragranular composition. In an aspect, the lubricant is present in the extragranular composition. In an aspect, the lubricant is present in the intragranular composition, and said intragranular composition is prepared by roller compaction. As used in this disclosure, a lubricant refers to a pharmaceutical excipient added to a tablet formulation which reduces friction at the tablet's surface. In embodiments, the lubricant can reduce friction between a tablet's surface and processing equipment, e.g., between a tablet's surface and the wall of a die cavity in which a tablet is formed. Therefore, a lubricant can reduce friction between a die wall and the granules of the formulation as the tablet is formed and ejected. Pharmaceutically acceptable lubricants are non-toxic and pharmacologically inactive substances. Further, the lubricants can be water soluble or water insoluble, although they preferably are water soluble in the presently disclosed drug delivery systems.

In an embodiment, the lubricant comprises or is magnesium stearate. In some embodiments, the lubricant comprises or is sodium stearyl fumarate. In some embodiments, the lubricant comprises or is polyethylene glycol (e.g., PEG8K).

In an aspect, the lubricant can comprise or can be selected from, for example, a fatty acid, a fatty acid salt, a fatty acid ester, talc, a glyceride ester, a metal silicate, or any combination thereof. In embodiments, the lubricant can comprise or can be selected from magnesium stearate, stearic acid, magnesium silicate, aluminum silicate, isopropyl myristate, sodium oleate, sodium stearoyl lactate, sodium stearoyl fumarate, titanium dioxide, or combinations thereof. Examples of water soluble lubricants include but are not limited to leucine, sodium lauryl sulfate, sucrose stearate, boric acid, sodium acetate, sodium oleate, sodium stearyl fumarate, and PEG. In another aspect, the total concentration of the lubricant in the solid pharmaceutical composition can be from 0.05 wt % to 5 wt %, 0.1 wt % to 3 wt %, 1 wt % to 2 wt %, or about 1.5 wt % or about 2.5 wt %. In another aspect, the total concentration of the lubricant in the solid pharmaceutical composition can be from 0.05 wt % to about 5 wt %, from about 1 wt % to about 5 wt %, or about 2.5%.

### Osmotic Agents

The drug, or certain salt forms of the drug, may itself function as an osmotic agent, and a drug formulation with such a drug, or salt form thereof, optionally may include one or more additional pharmaceutically acceptable osmotic agents that are pharmacologically inactive substances. Pharmaceutical excipients for the erdafitinib solid pharmaceutical composition may include one or more osmotic agents. The one or more osmotic agents can be present in the solid pharmaceutical composition as a component of the intragranular solid composition, the extragranular solid composition, or both the intragranular and extragranular solid compositions. Examples of osmotic agents include but are limited to inorganic salts and carbohydrates, such as urea, potassium chloride, sodium chloride, sucrose, fructose, lactose, and mannitol.

In an embodiment, the drug itself, in particular erdafitinib lactate, in particular erdafitinib mono L-lactate, functions as an osmotic agent.

### Methods of Preparing Tablet Compositions

In one aspect, provided is a process for making a solid pharmaceutical composition, such as a tablet, particularly a minitablet. In some embodiments, the process comprises preparing an intragranular solid composition comprising erdafitinib L-lactate and at least one intragranular pharmaceutical excipient. In some embodiments, the intragranular pharmaceutical excipient comprises a binder. The binder may be any binder as described herein. For instance, the binder may be hydroxypropyl methyl cellulose. In some embodiments, the process comprises combining the intragranular solid composition with at least one extragranular pharmaceutical excipient to form a blend. In some embodiments, the intragranular excipient and the extragranular excipient do not comprise a common excipient. In some embodiments, the extragranular excipient comprises one or more of a binder, filler, glidant, and/or lubricant. Each of the extragranular binder, filler, glidant, and/or lubricant may be any binder, filler, glidant, and/or lubricant as described herein, respectively. In some embodiments, the extragranular binder comprises vinyl pyrrolidone-vinyl acetate (PVP VA). In some embodiments, the extragranular filler comprises microcrystalline cellulose, silicified microcrystalline cellulose, or a combination thereof. In some embodiments, the extragranular filler comprises microcrystalline cellulose. In some embodiments, the extragranular filler comprises a combination of microcrystalline cellulose and silicified microcrystalline cellulose. In some embodiments, the extragranular glidant may be any glidant as described herein. In some embodiments, the extragranular glidant comprises colloidal silicon dioxide (hydrophilic). In some embodiments, the extragranular lubricant may be any lubricant as described herein. In some embodiments, the extragranular lubricant comprises magnesium stearate. In some embodiments, the extragranular lubricant comprises sodium stearyl fumarate in some embodiments, the extragranular lubricant comprises polyethylene glycol. In some embodiments, the process comprises tableting the blend to form the solid pharmaceutical composition. In some embodiments, tableting is characterized by an ejection force below about 1000 N.

### Osmotic Drug Delivery Systems

Drug delivery systems particularly suitable for the effective release of drug formulations containing erdafitinib, such as those described in detail above, are described herein. These particular systems utilize osmotic pressure to drive controlled release of the drug through one or more apertures in the system housing. The terms “aperture” and “orifice” may be used interchangeably with “opening.”

In certain embodiments, the system includes a water-permeable, drug-impermeable polymer component that forms the housing. For example, the polymer component may be formed of a biocompatible elastomeric composition, such as a silicone or a thermoplastic polyurethane composition, that has the desired mechanical properties (e.g., soft, elastically flexible). The polymer component may have a tubular structure, for example in a dual lumen tube.

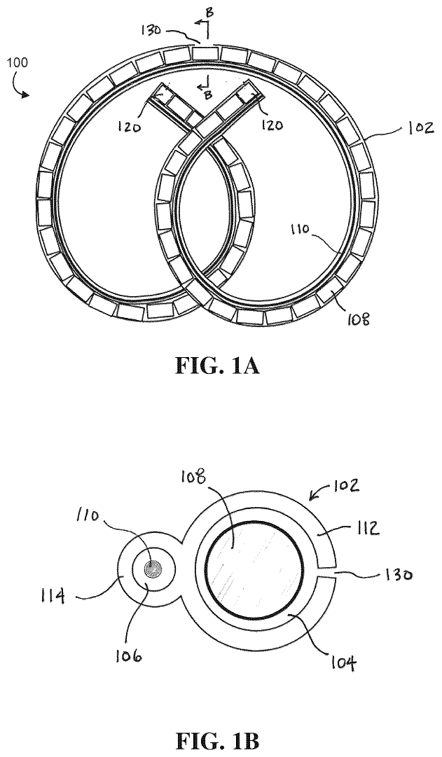

In one aspect, as shown in FIG. 1A-1B, a drug delivery system 100 includes a flexible device body 102 that defines a drug reservoir lumen 104 and a retention frame lumen 106. The drug reservoir lumen 104 contains a plurality of solid drug tablets 108 arranged end-to-end within the drug reservoir lumen. In some embodiments, including as illustrated, the drug tablets 108 have circular flat end faces and a cylindrical side wall. The retention frame lumen 106 houses an elastic wire, or retention frame, 110. The retention frame may be a superelastic alloy, such as nitinol. Silicone spacers 120 are secured in the ends of the drug reservoir lumen 104, for example using a silicone adhesive, to close/plug the drug reservoir lumen 104 at its ends and retain the drug tablets 108 therein.

As shown in FIG. 1B, the device body 102 includes a tubular wall structure 112 that defines the drug reservoir lumen 104 and a smaller tubular wall structure 114 that defines the retention frame lumen 106. The wall structures 112, 114 and lumens 104, 106 are substantially cylindrical as shown, although other variations of the shapes are possible.

In a preferred embodiment, the tubular wall structures 112, 114 are formed of silicone (MED-4750) and the tubular wall structure 112 has a wall thickness of 0.1 mm to 0.5 mm, or 0.15 mm to 0.4 mm, or 0.18 mm to 0.3 mm, or 0.2 mm. In some embodiments, the tubular wall structure 112 that defines the drug reservoir lumen 104 may be formed of another elastomeric material that is impermeable to the drug. As used herein, the term “impermeable to the drug” or “drug-impermeable” refers to the housing being substantially impermeable to the solubilized drug, e.g., erdafitinib, such that no substantial amount of the solubilized drug can diffuse therethrough over the therapeutic period in which the system is located in vivo.

The device body 102 includes an aperture 130 extending through the sidewall of tubular wall structure 112 that defines the drug reservoir lumen 104. In this illustrated embodiment, the device body has a single drug-release aperture. In exemplary embodiments, the diameter of the aperture is between about 20 μm and about 500 μm, such as between about 25 μm and about 300 μm, and more particularly between about 30 μm and about 200 μm. In one example, the aperture has a diameter between about 100 μm and about 200 μm, such as about 150 μm. In some other embodiments, the device body may have two or more apertures.

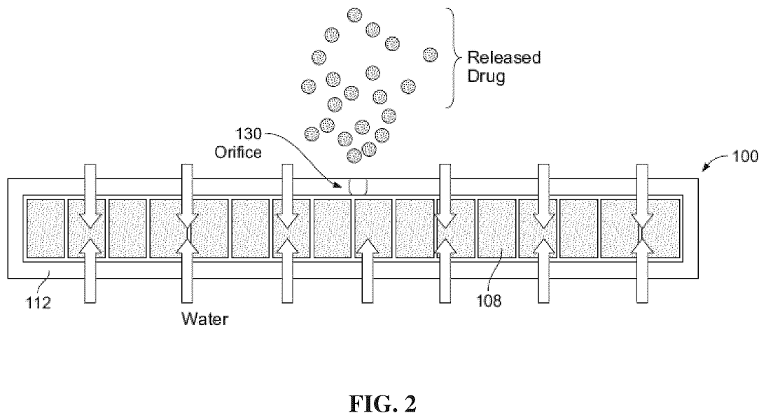

As illustrated in FIG. 2, the drug delivery system 100 in vivo operates as an osmotic pump. Water, or urine, from the patient's body diffuses through tubular wall structure 112 to contact and solubilize the drug tablets 108, creating an osmotic pressure to drive the solubilized drug from the drug delivery system through aperture (orifice) 130.

In some embodiments, the system includes a single aperture, i.e., a through-hole, located in a side wall of the tubular housing.

In some embodiments, the system is a closed structure configured to release the drug through the transient formation of microchannels defined between and at the interface of two surfaces, and/or of two components, in the system, as described in U.S. Pat. No. 9,814,671, which is incorporated by reference herein in its entirety.

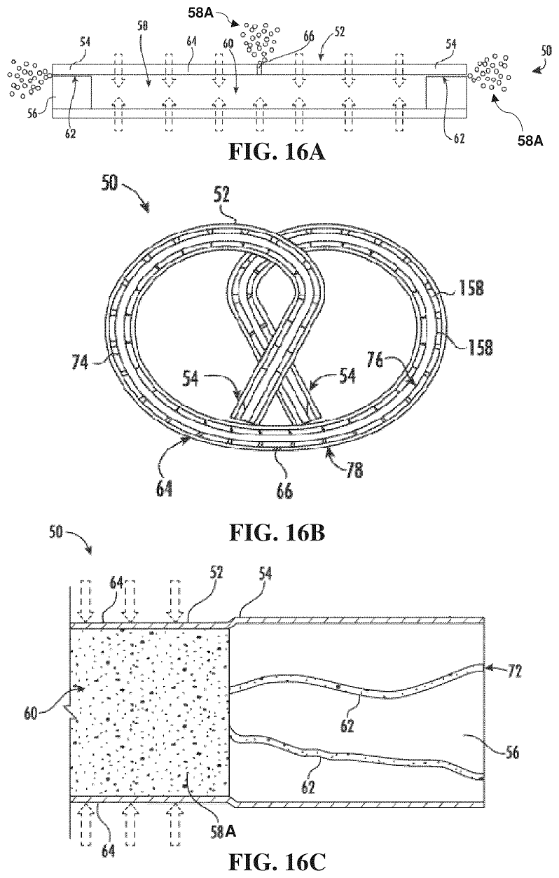

In some particular embodiments, the system includes both a through-hole in the tubular housing and housing structures configured to release the drug through the transient formation of microchannels defined between and at the interface of two surfaces, and/or of two components, as described in U.S. Pat. No. 11,020,575, which is incorporated by reference herein in its entirety. In some embodiments, the microchannels form in response to hydrostatic pressure that accumulates in the water-permeable body due to osmotically driven water influx. When the hydrostatic pressure increases above a certain threshold, the microchannels form, thereby forcing at least a portion of drug out of the device and relieving the hydrostatic pressure accumulation in the drug reservoir. The microchannel may collapse at least partially as the hydrostatic pressure has been relieved. The process repeats itself until all or a substantial portion of the drug has been released, or the osmotically driven water influx is insufficient to continue the process. As illustrated in FIG. 16A and FIG. 16C, drug delivery device 50 includes a device body 52 having a water-permeable wall portion 64 bounding a reservoir 60 (also referred to herein as a “reservoir lumen” or a “drug reservoir lumen”) containing a payload 58, such as a drug formulation (e.g., mini-tablets). The water-permeable wall portion 64 may generally be configured to permit water to enter the device and contact the drug formulation (i.e., payload) 58 located in the reservoir 60, to facilitate release of the fluidized drug 58A from the device. For example, osmotically driven water influx into the reservoir 60 may generate a pressure within the reservoir 60 that drives release of the fluidized drug 58A from the reservoir 60 via one or more mechanisms. For example, in embodiments described herein, release of the fluidized drug 58A from the device may occur through one or more preformed sidewall orifices 66 and/or through the transient formation of one or more microchannels 62 leading to an opening in an end of the device. For example, the device 50 may further include an elastic portion 54 that surrounds a restraining plug 56, and controls release of the drug from the device by the transient formation of one or more microchannels 62 between 54 and 56. As indicated by the dashed line arrows, water diffuses through the water-permeable wall 64 of the body 52 and enters the drug reservoir 60, forming fluidized drug 58A, which for example may be an aqueous solution comprising the drug provided in the payload 58 initially loaded in the reservoir 60. Hydrostatic pressure in the reservoir 60 causes the fluidized drug 58A to be pushed out of the reservoir 60 between the elastic portion 54 and the restraining plug 56, through microchannels 62 that are formed therebetween, for example by elastic deformation of one or both of the interfacing surfaces. In certain embodiments described herein, combinations of these release mechanisms are employed to provide the desired drug release profile.

As illustrated in FIG. 16B, the device 50 of FIG. 16A is shown in a plan view, in a relatively expanded shape suited for retention in the body, e.g., in the urinary bladder. The device includes a water-permeable body 52 having a drug reservoir portion 78 and a retention frame portion 76. The retention frame portion 76 may include a retention frame 74 that is deformable between a relatively expanded shape and a relatively lower-profile shape suitable for insertion into the bladder via a catheter. In some embodiments, the retention frame 74 includes or consists of an elastic wire. For example, the retention frame 74 is an elastic wire formed from a superelastic alloy, such as nitinol. The drug reservoir lumen may be loaded with a number of drug units 158 in a serial arrangement. The drug units may be tablets, such as mini-tablets. As used herein, the term “drug reservoir portion” refers to the portion of the device that forms and defines the “drug reservoir” or “drug reservoir lumen”. For the purposes of this disclosure, the term “retention shape” generally denotes any shape suited for retaining the device in the bladder, including but not limited to a coiled or “pretzel” shape as shown in FIG. 16B. The retention shape enables the device to resist becoming entrained in urine and excreted when the patient voids. The terms “relatively expanded shape”, “relatively higher-profile shape” may be used interchangeably with “retention shape.”

Referring again to FIG. 2, the device body 102 may be formed by a molding or extrusion process, or an additive manufacturing process. In some embodiments, where the drug constituent and device constituent are produced separately and then combined, the drug constituent (e.g., tablets) is loaded into the drug reservoir lumen and then the drug reservoir lumen is closed at its ends, e.g., with a silicone spacer and/or adhesive. In some other embodiments, the drug constituent and device constituent are produced together, such as in an additive manufacturing process, e.g., a 3-D printing process as known in the art.

In embodiments, the systems are configured for intravesical insertion and retention in a patient. In a preferred embodiment, the system is elastically deformable between a low-profile deployment shape (e.g., a relatively straightened shape) suited for insertion through the urethra of a patient and into the patient's bladder and a relatively expanded retention shape (e.g., pretzel shape, bi-oval coil shape, S-shape, etc.) suited for retention within the bladder. For example, the housing or tube of the system may have two opposing free ends, which are directed away from one another when the system is in a low-profile deployment shape and which are directed toward one another when the system is in a relatively expanded retention shape. The relatively expanded shape may include a pair of overlapping coils, sometime referred to as a “pretzel” shape. In particular embodiments, the ends of the elongated system generally lie within the boundaries of a bi-oval-like expanded retention shape.

In the bladder, the system should be compliant (e.g., easily flexed, soft feeling) during detrusor muscle contraction in order to avoid or mitigate discomfort and irritation to the patient. For example, the system may be configured for tolerability based on bladder characteristics and design considerations described in U.S. Pat. No. 11,065,426, which is incorporated herein by reference.

In some embodiments, the system includes retention frame lumen, and in certain of these embodiments, the retention frame lumen includes a retention frame, i.e., an elastic wire, such a nitinol wire. In certain other embodiments, the retention frame lumen is filled with a shape set elastic polymer. In some other embodiments, the system does not include a retention frame lumen or a retention frame or wire. Instead, the material of the housing is configured to be elastically deformable between the straightened shape and the retention shape, in the absence of a retention frame or wire. For example, in certain embodiments, the tubular housing is thermally shape set to have the coiled or other retention shape.

In embodiments, the tubular housing may be formed of a water permeable material. In a preferred embodiment, as described above with reference to the erdafitinib solid formulations, the drug is in a solid form (e.g., a tablet or plurality of tablets) and the tubular body is water permeable to permit in vivo solubilization of the drug while in the drug reservoir lumen. In some other embodiments, the solid formulation may be in a flowable particulate form, such as beads, granules, powder, or the like.

In some embodiments, the material for the wall structure of the present systems is selected from silicone and suitable thermoplastic polyurethane (TPU)-based materials, known in the art. In some embodiments, the material for the wall structure is platinum cured silicone elastomer.

In one aspect, the system housing is in the form of an annulus, i.e., a cylindrical tube. In one embodiment, an inner diameter of the cylindrical tube may be from 1.0 mm to 3.0 mm, or from 2.0 mm to 3.0 mm, or from 2.2 mm to 2.8 mm, or 2.6 mm to 2.7 mm, or 2.64 mm. In one embodiment, an outer diameter of the cylindrical tube is from about 2.0 mm to about 4.1 mm. In one embodiment, a thickness of the wall structure (the annulus) of the cylindrical tube is from about 0.2 mm to about 1.0 mm.

In embodiments, the systems described herein are configured to release a therapeutically effective amount of the drug, where the rate of the release of the drug from the drug delivery system is zero order over at least 36 hours. In one embodiment, the rate of the release of the drug from the drug delivery system is essentially zero order over at least 7 days. In embodiments, the system is configured to release a therapeutically effective amount of the drug over a period from 2 days to 6 months, e.g., from 2 days to 90 days, from 7 days to 30 days, or from 7 days to 14 days. Desirably, the rate of the release of the drug from the drug delivery system is zero order over at least 7 days, e.g., from 7 to 14 days, or longer, such as up to 3 months or 90 days. In certain embodiments, the system is configured to begin release of the drug after a lag time.

In some embodiments, the system is configured to release the erdafitinib at an average rate of 1 mg/day to 10 mg/day, depending on the desired treatment regimen. In some embodiments, the system is configured to release the erdafitinib at an average rate of 1 mg/day to 2 mg/day. In some embodiments, the system is configured to release the erdafitinib at an average rate of 4 mg/day to 6 mg/day. In some embodiments, the system is configured to release the erdafitinib at an average rate of 2 mg/day or 4 mg/day. In some embodiments, the system is configured to release the erdafitinib with a zero order release profile.

The osmotic drug delivery system may be loaded with a solid form of the erdafitinib salt, such as the tablets described throughout this disclosure. For example, the system may have a drug reservoir lumen configured to hold in an elongated form several of the disclosed drug tablets in an end-to-end serial arrangement. In some embodiments, the system holds from about 10 to 100 cylindrical drug tablets (e.g, 44 tablets), such as mini-tablets, which may be serially loaded in the drug reservoir lumen. In some embodiments, the system holds from about 10 to 100 cylindrical drug tablets (e.g., from about 40 to about 50 tablets), such as mini-tablets, which may be serially loaded in the drug reservoir lumen. In some embodiments, each mini-tablet has a mass of from about 20 mg to about 30 mg, or of from about 22 mg to about 24 mg. In some embodiments, the formulation comprises mini-tablets having a total length of from about 14 cm to about 16 cm, of about 14.5 cm to about 15.5 cm, or of about 14.7 cm to about 14.8 cm. In some embodiments, the total tablet mass loaded within the system is from about 900 mg to about 1000 mg. In some embodiments, the total tablet mass loaded within the system is from about 920 mg to about 965 mg. In some embodiments, the total tablet mass loaded within the system is from about 920 mg to about 950 mg. In some embodiments, the system comprises from about 500 mg to about 750 mg of erdafitinib (free base equivalent).

In some embodiments, the drug delivery system comprises: a dual lumen system comprising silicone tubing with a 0.2 mm wall thickness, wherein the dual lumen system comprises (i) a retention frame lumen (“small lumen”) enclosing a wireform as a retentive feature, and (ii) a drug reservoir lumen (“large lumen”) having a 2.64 mm inner-diameter filled with a plurality of mini-tablets, wherein the silicone tubing surrounding the drug reservoir lumen comprises a single 150 μm orifice, and wherein the drug reservoir lumen is sealed by two end plugs (e.g., parylene coated end plugs) which are sealed to the silicone tubing surrounding the drug reservoir lumen (e.g., using silicone adhesive) and are capable of forming transient microchannels. The plurality of mini-tablets may have, for instance, a total mini-tablet length of about 15.0 cm and a total tablet mass of about 932 mg (e.g., 620 mg of erdafitinib (free base equivalent) when the mini-tablets comprise 80 wt % erdafitinib mono-L-lactate, 18 wt % microcrystalline cellulose, 0.5 wt % hydrophilic colloidal silicon dioxide, and 1.5 wt % magnesium stearate). The plurality of mini-tablets may have, for instance, a total mini-tablet length of about 15.0 cm and a total tablet mass of about 932 mg (e.g., 620 mg of erdafitinib (free base equivalent) when the mini-tablets comprise 80 wt % erdafitinib mono-L-lactate, intragranular; 18 wt % microcrystalline cellulose, intragranular; 0.5 wt % hydrophilic colloidal silicon dioxide, such as 0.3 wt % intragranular, plus 0.2 wt % extragranular; and 1.5 wt % magnesium stearate, such as 0.75 wt % intragranular and 0.75 wt % extragranular). In some embodiments, the drug delivery system exists in two shapes, one having a wireform in a low-profile (e.g., straightened) deployment shape, and another having a relatively expanded (e.g., pretzel) retention shape. In some embodiments, the drug delivery system is Prototype 4.

In some embodiments, the drug delivery system comprises: a dual lumen system comprising silicone tubing with a 0.2 mm wall thickness, wherein the dual lumen system comprises (i) a retention frame lumen (“small lumen”) enclosing a wireform as a retentive feature, and (ii) a drug reservoir lumen (“large lumen”) having a 2.64 mm inner-diameter filled with a plurality of mini-tablets, wherein the silicone tubing surrounding the drug reservoir lumen comprises a single 150 μm orifice, and wherein the drug reservoir lumen is sealed by two end plugs (e.g., parylene coated end plugs) which are sealed to the silicone tubing surrounding the drug reservoir lumen (e.g., using silicone adhesive) and are capable of forming transient microchannels. The plurality of mini-tablets may have, for instance, a total mini-tablet length of about 15.0 cm and a total tablet mass of about 961 mg (e.g., 726 mg of erdafitinib (free base equivalent) when the mini-tablets comprise 90.8 wt % erdafitinib mono-L-lactate, 4.5 wt % polyethylene glycol (e.g., PEG8K), and 4.7 wt % polyvinylpyrrolidone). The plurality of mini-tablets may have, for instance, a total mini-tablet length of about 15.0 cm and a total tablet mass of about 961 mg (e.g., 726 mg of erdafitinib (free base equivalent) when the mini-tablets comprise 90.8 wt % erdafitinib mono-L-lactate, intragranular; 4.5 wt % polyethylene glycol (e.g., PEG8K), extragranular; and 4.7 wt % polyvinylpyrrolidone, intragranular). In some embodiments, the drug delivery system exists in two shapes, one having a wireform in a low-profile (e.g., straightened) deployment shape, and another having a relatively expanded (e.g., pretzel) retention shape. In some embodiments, the drug delivery system is Prototype 5.

Also encompassed by this disclosure are variations of Prototype 4 and Prototype 5, wherein the mini-tablets may comprise any pharmaceutical formulation as described herein. In some embodiments, Prototype 4 comprises mini-tablets comprising 20 wt % water insoluble excipients. In some embodiments, Prototype 5 comprises mini-tablets comprising only water soluble excipients. In some embodiments, Prototype 4 or Prototype 5 may comprise any pharmaceutical formulation as described herein, including concept 1, concept 2, concept 3, concept 4, or concept 5.

As discussed herein with reference to the erdafitinib pharmaceutical formulations, the drug may be provided in a solid form suitable for being loaded within the drug reservoir lumen of the system. In some embodiments, the individual drug tablets may have essentially any selected shape and dimension that fits within the systems described herein. In one embodiment, the drug units are sized and shaped such that the drug reservoir lumens in the housings are substantially filled by a select number of the drug tablets. Each tablet may have a cross-sectional shape that substantially corresponds to a cross-sectional shape of the drug reservoir lumen of a particular housing. For example, the tablets may be substantially cylindrical in shape for positioning in a substantially cylindrical drug reservoir lumen.

In one embodiment, the drug units (tablets) are shaped to align in a row when the system is in its deployment configuration. For example, each drug unit may have a cross-sectional shape that corresponds to the cross-sectional shape of the drug reservoir lumens in the housing, and each drug unit may have end face shapes that correspond to the end faces of adjacent drug units. The interstices or breaks between drug units can accommodate deformation or movement of the system, such as during deployment, while permitting the individual drug units to retain their solid form. Thus, the drug delivery system may be relatively flexible or deformable despite being loaded with a solid drug, as each drug unit may be permitted to move with reference to adjacent drug units.

In embodiments, the drug units are “mini-tablets” that are suitably sized and shaped for insertion through a natural lumen of the body, such as the urethra. For the purpose of this disclosure, the term “mini-tablet” generally indicates a solid drug unit that is substantially cylindrical in shape, having end faces and a side face that is substantially cylindrical. The mini-tablet has a diameter, extending along the end face, in the range of about 1.0 to about 3.2 mm, such as between about 1.5 and about 3.1 mm. The mini-tablet has a length, extending along the side face, in the range of about 1.7 mm to about 4.8 mm, such as between about 2.0 mm and about 4.5 mm. The friability of the tablet may be less than about 2%.

### Methods of Drug Delivery and Treatment

The systems and methods disclosed herein may be adapted for use in humans or for use in veterinary or livestock applications. Accordingly, the term “patient” may refer to a human or other mammalian subject. In an embodiment, the patient is a human subject. The patient may be a cancer patient.

In certain embodiments, methods of treatment of urothelial cancers, such as bladder cancers, are provided herein. In certain embodiments, use of a drug delivery system as described herein for the manufacture of a medicament for the treatment of urothelial cancers, such as bladder cancers, are provided herein. In certain embodiments, a drug delivery system as described herein for use in the treatment of urothelial cancers, such as bladder cancers, are provided herein. In certain embodiments, erdafitinib for use in a drug delivery system as described herein for the treatment of urothelial cancers, such as bladder cancers, are provided herein. The methods or uses may include locally delivering or administering erdafitinib (such as in any of the formulations described herein) into the bladder of a patient in need of treatment, in particular a cancer patient, in an amount effective for the treatment of bladder cancer (e.g., from about 1-10 mg/day, as described herein). For example, the treatment may be effective at treating muscle invasive bladder cancer (MIBC), non-muscle invasive bladder cancer (NMIBC), and/or bacillus Calmette-Guerin (BCG)-naïve bladder cancer. In an aspect the patient, in particular a human, is a BCG-experienced bladder or NMIBC or MIBC cancer patient. In an aspect the patient, in particular a human, is a BCG-naïve bladder or NMIBC or MIBC cancer patient. In an aspect the patient, in particular a human, is a recurrent, bacillus Calmette-Guerin (BCG)-experienced high-risk papillary-only NMIBC (high-grade Ta/Tl) cancer patient, refusing or ineligible for radical cystectomy (RCy) In an aspect the patient, in particular a human, is a recurrent, BCG-experienced high-risk papillary-only NMIBC (high-grade Ta/Ti) cancer patient, scheduled for RCy. In an aspect the patient, in particular a human, is a recurrent, intermediate-risk NMIBC (Ta and Ti) cancer patient with a previous history of only low-grade disease. In an aspect the patient, in particular a human, is a MIBC cancer patient scheduled for RCy who has refused or is ineligible for cisplatin-based neoadjuvant chemotherapy.

In certain embodiments, the urothelial cancers as described herein are susceptible to an FGFR2 genetic alteration and/or an FGFR3 genetic alteration.

As used herein, “FGFR genetic alteration” refers to an alteration in the wild type FGFR gene, including, but not limited to, FGFR fusion genes, FGFR mutations, FGFR amplifications, or any combination thereof, in particular FGFR fusion genes, FGFR mutations, or any combination thereof. In certain embodiments, the FGFR2 or FGFR3 genetic alteration is an FGFR gene fusion. “FGFR fusion” or “FGFR gene fusion” refers to a gene encoding a portion of FGFR (e.g., FGRF2 or FGFR3) and one of the herein disclosed fusion partners, or a portion thereof, created by a translocation between the two genes. The terms “fusion” and “translocation” are used interchangeable herein. The presence of one or more of the following FGFR fusion genes in a biological sample from a patient can be determined using the disclosed methods or uses or by methods known to those of ordinary skill in the art: FGFR3-TACC3, FGFR3-BATAP2L1, FGFR2-BICC1, FGFR2-CASP7, or any combination thereof. In certain embodiments, FGFR3-TACC3 is FGFR3-TACC3 variant 1 (FGFR3-TACC3 V1) or FGFR3-TACC3 variant 3 (FGFR3-TACC3 V3). Table A provides the FGFR fusion genes and the FGFR and fusion partner exons that are fused. The sequences of the individual FGFR fusion genes are disclosed in Table A2. The underlined sequences correspond to either FGFR3 or FGFR2, the other sequences represent the fusion partners.

TABLE A

| Fusion Gene | FGFR Exon | Partner Exon |
|---|---|---|
| FGFR2 | | |
| FGFR2-BICC1 | 19 | 3 |
| FGFR2-CASP7 | 19 | 4 |
| FGFR3 | | |
| FGFR3-BAIAP2L1 | 18 | 2 |
| FGFR3-TACC3 V1 | 18 | 11 |
| FGFR3-TACC3 V3 | 18 | 10 |

TABLE A2

| | |
|---|---|
| FGFR3-TACC3 V1 | >ATGGGCGCCCCTGCCTGCGCCCTCGCGCTCTGCGTGGCCGTGGCCATCGTGGCC |
| (2850 base pairs) | GGCGCCTCCTCGGAGTCCTTGGGGACGGAGCAGCGCGTCGTGGGGCGAGCGGCA |
| (SEQ ID NO: 33) | GAAGTCCCGGGCCCAGAGCCCGGCCAGCAGGAGCAGTTGGTCTTCGGCAGCGGG |
| | GATGCTGTGGAGCTGAGCTGTCCCCCGCCCGGGGGTGGTCCCATGGGGCCCACTG |
| | TCTGGGTCAAGGATGGCACAGGGCTGGTGCCCTCGGAGCGTGTCCTGGTGGGGC |
| | CCCAGCGGCTGCAGGTGCTGAATGCCTCCCACGAGGACTCCGGGGCCTACAGCT |
| | GCCGGCAGCGGCTCACGCAGCGCGTACTGTGCCACTTCAGTGTGCGGGTGACAG |
| | ACGCTCCATCCTCGGGAGATGACGAAGACGGGGAGGACGAGGCTGAGGACACA |
| | GGTGTGGACACAGGGGCCCCTTACTGGACACGGCCCGAGCGGATGGACAAGAAG |
| | CTGCTGGCCGTGCCGGCCGCCAACACCGTCCGCTTCCGCTGCCCAGCCGCTGGCA |
| | ACCCCACTCCCTCCATCTCCTGGCTGAAGAACGGCAGGGAGTTCCGCGGCGAGC |
| | ACCGCATTGGAGGCATCAAGCTGCGGCATCAGCAGTGGAGCCTGGTCATGGAAA |
| | GCGTGGTGCCCTCGGACCGCGGCAACTACACCTGCGTCGTGGAGAACAAGTTTG |
| | GCAGCATCCGGCAGACGTACACGCTGGACGTGCTGGAGCGCTCCCCGCACCGGC |
| | CCATCCTGCAGGCGGGGCTGCCGGCCAACCAGACGGCGGTGCTGGGCAGCGACG |
| | TGGAGTTCCACTGCAAGGTGTACAGTGACGCACAGCCCCACATCCAGTGGCTCA |
| | AGCACGTGGAGGTGAATGGCAGCAAGGTGGGCCCGGACGGCACACCCTACGTTA |
| | CCGTGCTCAAGACGGGGGCGCTAACACCACCGACAAGGAGCTAGAGGTTCTCT |
| | CCTTGCACAACGTCACCTTTGAGGACGCCGGGGAGTACACCTGCCTGGCGGGCA |
| | ATTCTATTGGGTTTTCTCATCACTCTGCGTGGCTGGTGGTGCTGCCAGCCGAGGA |
| | GGAGCTGGTGGAGGCTGACGAGGCGGGCAGTGTGTATGCAGGCATCCTCAGCTA |
| | CGGGGTGGGCTTCTTCCTGTTCATCCTGGTGGTGGCGGCTGTGACGCTCTGCCGC |
| | CTGCGCAGCCCCCCCAAGAAAGGCCTGGGCTCCCCCACCGTGCACAAGATCTCCC |
| | GCTTCCCGCTCAAGCGACAGGTGTCCCTGGAGTCCAACGCGTCCATGAGCTCCAA |
| | CACACCACTGGTGCGCATCGCAAGGCTGTCCTCAGGGGAGGGCCCCACGCTGGC |
| | CAATGTCTCCGAGCTCGAGCTGCCTGCCGACCCCAAATGGGAGCTGTCTCGGGCC |
| | CGGCTGACCCTGGGCAAGCCCCTTGGGGAGGGCTGCTTCGGCCAGGTGGTCATG |
| | GCGGAGGCCATCGGCATTGACAAGGACCGGGCCGCCAAGCCTGTCACCGTAGCC |
| | GTGAAGATGCTGAAAGACGATGCCACTGACAAGGACCTGTCGGACCTGGTGTCT |
| | GAGATGGAGATGATGAAGATGATCGGGAAACACAAAAACATCATCAACCTGCTG |
| | GGCGCCTGCACGCAGGGCGGGCCCCTGTACGTGCTGGTGGAGTACGCGGCCAAG |
| | GGTAACCTGCGGGAGTTTCTGCGGGCGCGGCGGCCCCCGGGCCTGGACTACTCCT |
| | TCGACACCTGCAAGCCGCCCGAGGAGCAGCTCACCTTCAAGGACCTGGTGTCCTG |
| | TGCCTACCAGGTGGCCCGGGGCATGGAGTACTTGGCCTCCCAGAAGTGCATCCAC |
| | AGGGACCTGGCTGCCCGCAATGTGCTGGTGACCGAGGACAACGTGATGAAGATC |
| | GCAGACTTCGGGCTGGCCCGGGACGTGCACAACCTCGACTACTACAAGAAGACG |
| | ACCAACGGCCGGCTGCCCGTGAAGTGGATGGCGCCTGAGGCCTTGTTTGACCGA |
| | GTCTACACTCACCAGAGTGACGTCTGGTCCTTTGGGGTCCTGCTCTGGGAGATCT |
| | TCACGCTGGGGGGCTCCCCGTACCCCGGCATCCCTGTGGAGGAGCTCTTCAAGCT |
| | GCTGAAGGAGGGCCACCGCATGGACAAGCCCGCCAACTGCACACACGACCTGTA |
| | CATGATCATGCGGGAGTGCTGGCATGCCGCGCCCTCCCAGAGGCCCACCTTCAAG |
| | CAGCTGGTGGAGGACCTGGACCGTGTCCTTACCGTGACGTCCACCGACGTAAAG |
| | GCGACACAGGAGGAGAACCGGGAGCTGAGGAGCAGGTGTGAGGAGCTCCACGG |
| | GAAGAACCTGGAACTGGGGAAGATCATGGACAGGTTCGAAGAGGTTGTGTACCA |
| | GGCCATGGAGGAAGTTCAGAAGCAGAAGGAACTTTCCAAAGCTGAAATCCAGAA |
| | AGTTCTAAAAGAAAAAGACCAACTTACCACAGATCTGAACTCCATGGAGAAGTC |
| | CTTCTCCGACCTCTTCAAGCGTTTTGAGAAACAGAAAGAGGTGATCGAGGGCTAC |
| | CGCAAGAACGAAGAGTCACTGAAGAAGTGCGTGGAGGATTACCTGGCAAGGATC |
| | ACCCAGGAGGGCCAGAGGTACCAAGCCCTGAAGGCCCACGCGGAGGAGAAGCT |
| | GCAGCTGGCAAACGAGGAGATCGCCCAGGTCCGGAGCAAGGCCCAGGCGGAAG |
| | CGTTGGCCCTCCAGGCCAGCCTGAGGAAGGAGCAGATGCGCATCCAGTCGCTGG |
| | AGAAGACAGTGGAGCAGAAGACTAAAGAGAACGAGGAGCTGACCAGGATCTGC |
| | GACGACCTCATCTCCAAGATGGAGAAGATCTGA |
| FGFR3-TACC3 V3 | >ATGGGCGCCCCTGCCTGCGCCCTCGCGCTCTGCGTGGCCGTGGCCATCGTGGCC |
| (2955 base pairs) | GGCGCCTCCTCGGAGTCCTTGGGGACGGAGCAGCGCGTCGTGGGGCGAGCGGCA |
| (SEQ ID NO: 34 | GAAGTCCCGGGCCCAGAGCCCGGCCAGCAGGAGCAGTTGGTCTTCGGCAGCGGG |
| | GATGCTGTGGAGCTGAGCTGTCCCCCGCCCGGGGGTGGTCCCATGGGGCCCACTG |
| | TCTGGGTCAAGGATGGCACAGGGCTGGTGCCCTCGGAGCGTGTCCTGGTGGGGC |
| | CCCAGCGGCTGCAGGTGCTGAATGCCTCCCACGAGGACTCCGGGGCCTACAGCT |
| | GCCGGCAGCGGCTCACGCAGCGCGTACTGTGCCACTTCAGTGTGCGGGTGACAG |
| | ACGCTCCATCCTCGGGAGATGACGAAGACGGGGAGGACGAGGCTGAGGACACA |
| | GGTGTGGACACAGGGGCCCCTTACTGGACACGGCCCGAGCGGATGGACAAGAAG |
| | CTGCTGGCCGTGCCGGCCGCCAACACCGTCCGCTTCCGCTGCCCAGCCGCTGGCA |
| | ACCCCACTCCCTCCATCTCCTGGCTGAAGAACGGCAGGGAGTTCCGCGGCGAGC |
| | ACCGCATTGGAGGCATCAAGCTGCGGCATCAGCAGTGGAGCCTGGTCATGGAAA |
| | GCGTGGTGCCCTCGGACCGCGGCAACTACACCTGCGTCGTGGAGAACAAGTTTG |
| | GCAGCATCCGGCAGACGTACACGCTGGACGTGCTGGAGCGCTCCCCGCACCGGC |
| | CCATCCTGCAGGCGGGGCTGCCGGCCAACCAGACGGCGGTGCTGGGCAGCGACG |
| | TGGAGTTCCACTGCAAGGTGTACAGTGACGCACAGCCCCACATCCAGTGGCTCA |
| | AGCACGTGGAGGTGAATGGCAGCAAGGTGGGCCCGGACGGCACACCCTACGTTA |
| | CCGTGCTCAAGACGGCGGGCGCTAACACCACCGACAAGGAGCTAGAGGTTCTCT |
| | CCTTGCACAACGTCACCTTTGAGGACGCCGGGGAGTACACCTGCCTGGCGGGCA |
| | ATTCTATTGGGTTTTCTCATCACTCTGCGTGGCTGGTGGTGCTGCCAGCCGAGGA |
| | GGAGCTGGTGGAGGCTGACGAGGCGGGCAGTGTGTATGCAGGCATCCTCAGCTA |
| | CGGGGTGGGCTTCTTCCTGTTCATCCTGGTGGTGGCGGCTGTGACGCTCTGCCGC |
| | CTGCGCAGCCCCCCCAAGAAAGGCCTGGGCTCCCCCACCGTGCACAAGATCTCCC |
| | GCTTCCCGCTCAAGCGACAGGTGTCCCTGGAGTCCAACGCGTCCATGAGCTCCAA |
| | CACACCACTGGTGCGCATCGCAAGGCTGTCCTCAGGGGAGGGCCCCACGCTGGC |
| | CAATGTCTCCGAGCTCGAGCTGCCTGCCGACCCCAAATGGGAGCTGTCTCGGGCC |
| | CGGCTGACCCTGGGCAAGCCCCTTGGGGAGGGCTGCTTCGGCCAGGTGGTCATG |
| | GCGGAGGCCATCGGCATTGACAAGGACCGGGCCGCCAAGCCTGTCACCGTAGCC |
| | GTGAAGATGCTGAAAGACGATGCCACTGACAAGGACCTGTCGGACCTGGTGTCT |
| | GAGATGGAGATGATGAAGATGATCGGGAAACACAAAAACATCATCAACCTGCTG |
| | GGCGCCTGCACGCAGGGCGGGCCCCTGTACGTGCTGGTGGAGTACGCGGCCAAG |
| | GGTAACCTGCGGGAGTTTCTGCGGGCGCGCGGCCCCGGGCCTGGACTACTCCT |
| | TCGACACCTGCAAGCCGCCCGAGGAGCAGCTCACCTTCAAGGACCTGGTGTCCTG |
| | TGCCTACCAGGTGGCCCGGGGCATGGAGTACTTGGCCTCCCAGAAGTGCATCCAC |
| | AGGGACCTGGCTGCCCGCAATGTGCTGGTGACCGAGGACAACGTGATGAAGATC |
| | GCAGACTTCGGGCTGGCCCGGGACGTGCACAACCTCGACTACTACAAGAAGACG |
| | ACCAACGGCCGGCTGCCCGTGAAGTGGATGGCGCCTGAGGCCTTGTTTGACCGA |
| | GTCTACACTCACCAGAGTGACGTCTGGTCCTTTGGGGTCCTGCTCTGGGAGATCT |
| | TCACGCTGGGGGGCTCCCCGTACCCCGGCATCCCTGTGGAGGAGCTCTTCAAGCT |
| | GCTGAAGGAGGGCCACCGCATGGACAAGCCCGCCAACTGCACACACGACCTGTA |
| | CATGATCATGCGGGAGTGCTGGCATGCCGCGCCCTCCCAGAGGCCCACCTTCAAG |
| | CAGCTGGTGGAGGACCTGGACCGTGTCCTTACCGTGACGTCCACCGACGTGCCAG |
| | GCCCACCCCCAGGTGTTCCCGCGCCTGGGGGCCCACCCCTGTCCACCGGACCTAT |
| | AGTGGACCTGCTCCAGTACAGCCAGAAGGACCTGGATGCAGTGGTAAAGGCGAC |
| | ACAGGAGGAGAACCGGGAGCTGAGGAGCAGGTGTGAGGAGCTCCACGGGAAGA |
| | ACCTGGAACTGGGGAAGATCATGGACAGGTTCGAAGAGGTTGTGTACCAGGCCA |
| | TGGAGGAAGTTCAGAAGCAGAAGGAACTTTCCAAAGCTGAAATCCAGAAAGTTC |
| | TAAAAGAAAAAGACCAACTTACCACAGATCTGAACTCCATGGAGAAGTCCTTCT |
| | CCGACCTCTTCAAGCGTTTTGAGAAACAGAAAGAGGTGATCGAGGGCTACCGCA |
| | AGAACGAAGAGTCACTGAAGAAGTGCGTGGAGGATTACCTGGCAAGGATCACCC |
| | AGGAGGGCCAGAGGTACCAAGCCCTGAAGGCCCACGCGGAGGAGAAGCTGCAG |
| | CTGGCAAACGAGGAGATCGCCCAGGTCCGGAGCAAGGCCCAGGCGGAAGCGTTG |
| | GCCCTCCAGGCCAGCCTGAGGAAGGAGCAGATGCGCATCCAGTCGCTGGAGAAG |
| | ACAGTGGAGCAGAAGACTAAAGAGAACGAGGAGCTGACCAGGATCTGCGACGA |
| | CCTCATCTCCAAGATGGAGAAGATCTGA |
| FGFR3-BAIAP2L1 | >ATGGGCGCCCCTGCCTGCGCCCTCGCGCTCTGCGTGGCCGTGGCCATCGTGGCC |
| (3765 base pairs) | GGCGCCTCCTCGGAGTCCTTGGGGACGGAGCAGCGCGTCGTGGGGCGAGCGGCA |
| (SEQ ID NO: 35) | GAAGTCCCGGGCCCAGAGCCCGGCCAGCAGGAGCAGTTGGTCTTCGGCAGCGGG |
| | GATGCTGTGGAGCTGAGCTGTCCCCCGCCCGGGGGTGGTCCCATGGGGCCCACTG |
| | TCTGGGTCAAGGATGGCACAGGGCTGGTGCCCTCGGAGCGTGTCCTGGTGGGGC |
| | CCCAGCGGCTGCAGGTGCTGAATGCCTCCCACGAGGACTCCGGGGCCTACAGCT |
| | GCCGGCAGCGGCTCACGCAGCGCGTACTGTGCCACTTCAGTGTGCGGGTGACAG |
| | ACGCTCCATCCTCGGGAGATGACGAAGACGGGGAGGACGAGGCTGAGGACACA |
| | GGTGTGGACACAGGGGCCCCTTACTGGACACGGCCCGAGCGGATGGACAAGAAG |
| | CTGCTGGCCGTGCCGGCCGCCAACACCGTCCGCTTCCGCTGCCCAGCCGCTGGCA |
| | ACCCCACTCCCTCCATCTCCTGGCTGAAGAACGGCAGGGAGTTCCGCGGCGAGC |
| | ACCGCATTGGAGGCATCAAGCTGCGGCATCAGCAGTGGAGCCTGGTCATGGAAA |
| | GCGTGGTGCCCTCGGACCGCGGCAACTACACCTGCGTCGTGGAGAACAAGTTTG |
| | GCAGCATCCGGCAGACGTACACGCTGGACGTGCTGGAGCGCTCCCCGCACCGGC |
| | CCATCCTGCAGGCGGGGCTGCCGGCCAACCAGACGGCGGTGCTGGGCAGCGACG |
| | TGGAGTTCCACTGCAAGGTGTACAGTGACGCACAGCCCCACATCCAGTGGCTCA |
| | AGCACGTGGAGGTGAATGGCAGCAAGGTGGGCCCGGACGGCACACCCTACGTTA |
| | CCGTGCTCAAGTCCTGGATCAGTGAGAGTGTGGAGGCCGACGTGCGCCTCCGCCT |
| | GGCCAATGTGTCGGAGCGGGACGGGGGCGAGTACCTCTGTCGAGCCACCAATTT |
| | CATAGGCGTGGCCGAGAAGGCCTTTTGGCTGAGCGTTCACGGGCCCCGAGCAGC |
| | CGAGGAGGAGCTGGTGGAGGCTGACGAGGGGGGCAGTGTGTATGCAGGCATCCT |
| | CAGCTACGGGGTGGGCTTCTTCCTGTTCATCCTGGTGGTGGCGGCTGTGACGCTC |
| | TGCCGCCTGCGCAGCCCCCCCAAGAAAGGCCTGGGCTCCCCCACCGTGCACAAG |
| | ATCTCCCGCTTCCCGCTCAAGCGACAGGTGTCCCTGGAGTCCAACGCGTCCATGA |
| | GCTCCAACACACCACTGGTGCGCATCGCAAGGCTGTCCTCAGGGGAGGGCCCCA |
| | CGCTGGCCAATGTCTCCGAGCTCGAGCTGCCTGCCGACCCCAAATGGGAGCTGTC |
| | TCGGGCCCGGCTGACCCTGGGCAAGCCCCTTGGGGAGGGCTGCTTCGGCCAGGT |
| | GGTCATGGCGGAGGCCATCGGCATTGACAAGGACCGGGCCGCCAAGCCTGTCAC |
| | CGTAGCCGTGAAGATGCTGAAAGACGATGCCACTGACAAGGACCTGTCGGACCT |
| | GGTGTCTGAGATGGAGATGATGAAGATGATCGGGAAACACAAAAACATCATCAA |
| | CCTGCTGGGCGCCTGCACGCAGGGCGGGCCCCTGTACGTGCTGGTGGAGTACGC |
| | GGCCAAGGGTAACCTGCGGGAGTTTCTGCGGGCGCGGCGGCCCCCGGGCCTGGA |
| | CTACTCCTTCGACACCTGCAAGCCGCCCGAGGAGCAGCTCACCTTCAAGGACCTG |
| | GTGTCCTGTGCCTACCAGGTGGCCCGGGGCATGGAGTACTTGGCCTCCCAGAAGT |
| | GCATCCACAGGGACCTGGCTGCCCGCAATGTGCTGGTGACCGAGGACAACGTGA |
| | TGAAGATCGCAGACTTCGGGCTGGCCCGGGACGTGCACAACCTCGACTACTACA |
| | AGAAGACGACCAACGGCCGGCTGCCCGTGAAGTGGATGGCGCCTGAGGCCTTGT |
| | TTGACCGAGTCTACACTCACCAGAGTGACGTCTGGTCCTTTGGGGTCCTGCTCTG |
| | GGAGATCTTCACGCTGGGGGGCTCCCCGTACCCCGGCATCCCTGTGGAGGAGCTC |
| | TTCAAGCTGCTGAAGGAGGGCCACCGCATGGACAAGCCCGCCAACTGCACACAC |
| | GACCTGTACATGATCATGCGGGAGTGCTGGCATGCCGCGCCCTCCCAGAGGCCC |
| | ACCTTCAAGCAGCTGGTGGAGGACCTGGACCGTGTCCTTACCGTGACGTCCACCG |
| | ACAATGTTATGGAACAGTTCAATCCTGGGCTGCGAAATTTAATAAACCTGGGGA |
| | AAAATTATGAGAAAGCTGTAAACGCTATGATCCTGGCAGGAAAAGCCTACTACG |
| | ATGGAGTGGCCAAGATCGGTGAGATTGCCACTGGGTCCCCCGTGTCAACTGAACT |
| | GGGACATGTCCTCATAGAGATTTCAAGTACCCACAAGAAACTCAACGAGAGTCT |
| | TGATGAAAATTTTAAAAAATTCCACAAAGAGATTATCCATGAGCTGGAGAAGAA |
| | GATAGAACTTGACGTGAAATATATGAACGCAACTCTAAAAAGATACCAAACAGA |
| | ACACAAGAATAAATTAGAGTCTTTGGAGAAATCCCAAGCTGAGTTGAAGAAGAT |
| | CAGAAGGAAAAGCCAAGGAAGCCGAAACGCACTCAAATATGAACACAAAGAAA |
| | TTGAGTATGTGGAGACCGTTACTTCTCGTCAGAGTGAAATCCAGAAATTCATTGC |
| | AGATGGTTGCAAAGAGGCTCTGCTTGAAGAGAAGAGGCGCTTCTGCTTTCTGGTT |
| | GATAAGCACTGTGGCTTTGCAAACCACATACATTATTATCACTTACAGTCTGCAG |
| | AACTACTGAATTCCAAGCTGCCTCGGTGGCAGGAGACCTGTGTTGATGCCATCAA |
| | AGTGCCAGAGAAAATCATGAATATGATCGAAGAAATAAAGACCCCAGCCTCTAC |
| | CCCCGTGTCTGGAACTCCTCAGGCTTCACCCATGATCGAGAGAAGCAATGTGGTT |
| | AGGAAAGATTACGACACCCTTTCTAAATGCTCACCAAAGATGCCCCCCGCTCCTT |
| | CAGGCAGAGCATATACCAGTCCCTTGATCGATATGTTTAATAACCCAGCCACGGC |
| | TGCCCCGAATTCACAAAGGGTAAATAATTCAACAGGTACTTCCGAAGATCCCAGT |
| | TTACAGCGATCAGTTTCGGTTGCAACGGGACTGAACATGATGAAGAAGCAGAAA |
| | GTGAAGACCATCTTCCCGCACACTGCGGGCTCCAACAAGACCTTACTCAGCTTTG |
| | CACAGGGAGATGTCATCACGCTGCTCATCCCCGAGGAGAAGGATGGCTGGCTCT |
| | ATGGAGAACACGACGTGTCCAAGGCGAGGGGTTGGTTCCCGTCGTCGTACACGA |
| | AGTTGCTGGAAGAAAATGAGACAGAAGCAGTGACCGTGCCCACGCCAAGCCCCA |
| | CACCAGTGAGAAGCATCAGCACCGTGAACTTGTCTGAGAATAGCAGTGTTGTCAT |
| | CCCCCCACCCGACTACTTGGAATGCTTGTCCATGGGGGCAGCTGCCGACAGGAG |
| | AGCAGATTCGGCCAGGACGACATCCACCTTTAAGGCCCCAGCGTCCAAGCCCGA |
| | GACCGCGGCTCCTAACGATGCCAACGGGACTGCAAAGCCGCCTTTTCTCAGCGG |
| | AGAAAACCCCTTTGCCACTGTGAAACTCCGCCCGACTGTGACGAATGATCGCTCG |
| | GCACCCATCATTCGATGA |
| FGFR2-BICC1 | >ATGGTCAGCTGGGGTCGTTTCATCTGCCTGGTCGTGGTCACCATGGCAACCTTGT |
| (4989 base pairs) | CCCTGGCCCGGCCCTCCTTCAGTTTAGTTGAGGATACCACATTAGAGCCAGAAGA |
| (SEQ ID NO: 36) | GCCACCAACCAAATACCAAATCTCTCAACCAGAAGTGTACGTGGCTGCGCCAGG |
| | GGAGTCGCTAGAGGTGCGCTGCCTGTTGAAAGATGCCGCCGTGATCAGTTGGACT |
| | AAGGATGGGGTGCACTTGGGGCCCAACAATAGGACAGTGCTTATTGGGGAGTAC |
| | TTGCAGATAAAGGGCGCCACGCCTAGAGACTCCGGCCTCTATGCTTGTACTGCCA |
| | GTAGGACTGTAGACAGTGAAACTTGGTACTTCATGGTGAATGTCACAGATGCCAT |
| | CTCATCCGGAGATGATGAGGATGACACCGATGGTGCGGAAGATTTTGTCAGTGA |
| | GAACAGTAACAACAAGAGAGCACCATACTGGACCAACACAGAAAAGATGGAAA |
| | AGCGGCTCCATGCTGTGCCTGCGGCCAACACTGTCAAGTTTCGCTGCCCAGCCGG |
| | GGGGAACCCAATGCCAACCATGCGGTGGCTGAAAAACGGGAAGGAGTTTAAGCA |
| | GGAGCATCGCATTGGAGGCTACAAGGTACGAAACCAGCACTGGAGCCTCATTAT |
| | GGAAAGTGTGGTCCCATCTGACAAGGGAAATTATACCTGTGTAGTGGAGAATGA |
| | ATACGGGTCCATCAATCACACGTACCACCTGGATGTTGTGGAGCGATCGCCTCAC |
| | CGGCCCATCCTCCAAGCCGGACTGCCGGCAAATGCCTCCACAGTGGTCGGAGGA |
| | GACGTAGAGTTTGTCTGCAAGGTTTACAGTGATGCCCAGCCCCACATCCAGTGGA |
| | TCAAGCACGTGGAAAAGAACGGCAGTAAATACGGGCCCGACGGGCTGCCCTACC |
| | TCAAGGTTCTCAAGGCCGCCGGTGTTAACACCACGGACAAAGAGATTGAGGTTC |
| | TCTATATTCGGAATGTAACTTTTGAGGACGCTGGGGAATATACGTGCTTGGCGGG |
| | TAATTCTATTGGGATATCCTTTCACTCTGCATGGTTGACAGTTCTGCCAGCGCCTG |
| | GAAGAGAAAAGGAGATTACAGCTTCCCCAGACTACCTGGAGATAGCCATTTACT |
| | GCATAGGGGTCTTCTTAATCGCCTGTATGGTGGTAACAGTCATCCTGTGCCGAAT |
| | GAAGAACACGACCAAGAAGCCAGACTTCAGCAGCCAGCCGGCTGTGCACAAGCT |
| | GACCAAACGTATCCCCCTGCGGAGACAGGTAACAGTTTCGGCTGAGTCCAGCTCC |
| | TCCATGAACTCCAACACCCCGCTGGTGAGGATAACAACACGCCTCTCTTCAACGG |
| | CAGACACCCCCATGCTGGCAGGGGTCTCCGAGTATGAACTTCCAGAGGACCCAA |
| | AATGGGAGTTTCCAAGAGATAAGCTGACACTGGGCAAGCCCCTGGGAGAAGGTT |
| | GCTTTGGGCAAGTGGTCATGGCGGAAGCAGTGGGAATTGACAAAGACAAGCCCA |
| | AGGAGGCGGTCACCGTGGCCGTGAAGATGTTGAAAGATGATGCCACAGAGAAAG |
| | ACCTTTCTGATCTGGTGTCAGAGATGGAGATGATGAAGATGATTGGGAAACACA |
| | AGAATATCATAAATCTTCTTGGAGCCTGCACACAGGATGGGCCTCTCTATGTCAT |
| | AGTTGAGTATGCCTCTAAAGGCAACCTCCGAGAATACCTCCGAGCCCGGAGGCC |
| | ACCCGGGATGGAGTACTCCTATGACATTAACCGTGTTCCTGAGGAGCAGATGACC |
| | TTCAAGGACTTGGTGTCATGCACCTACCAGCTGGCCAGAGGCATGGAGTACTTGG |
| | CTTCCCAAAAATGTATTCATCGAGATTTAGCAGCCAGAAATGTTTTGGTAACAGA |
| | AAACAATGTGATGAAAATAGCAGACTTTGGACTCGCCAGAGATATCAACAATAT |
| | AGACTATTACAAAAAGACCACCAATGGGCGGCTTCCAGTCAAGTGGATGGCTCC |
| | AGAAGCCCTGTTTGATAGAGTATACACTCATCAGAGTGATGTCTGGTCCTTCGGG |
| | GTGTTAATGTGGGAGATCTTCACTTTAGGGGGCTCGCCCTACCCAGGGATTCCCG |
| | TGGAGGAACTTTTTAAGCTGCTGAAGGAAGGACACAGAATGGATAAGCCAGCCA |
| | ACTGCACCAACGAACTGTACATGATGATGAGGGACTGTTGGCATGCAGTGCCCTC |
| | CCAGAGACCAACGTTCAAGCAGTTGGTAGAAGACTTGGATCGAATTCTCACTCTC |
| | ACAACCAATGAGATCATGGAGGAAACAAATACGCAGATTGCTTGGCCATCAAAA |
| | CTGAAGATCGGAGCCAAATCCAAGAAAGATCCCCATATTAAGGTTTCTGGAAAG |
| | AAAGAAGATGTTAAAGAAGCCAAGGAAATGATCATGTCTGTCTTAGACACAAAA |
| | AGCAATCGAGTCACACTGAAGATGGATGTTTCACATACAGAACATTCACATGTA |
| | ATCGGCAAAGGTGGCAACAATATTAAAAAAGTGATGGAAGAAACCGGATGCCAT |
| | ATCCACTTTCCAGATTCCAACAGGAATAACCAAGCAGAAAAAAGCAACCAGGTA |
| | TCTATAGCGGGACAACCAGCAGGAGTAGAATCTGCCCGAGTTAGAATTCGGGAG |
| | CTGCTTCCTTTGGTGCTGATGTTTGAGCTACCAATTGCTGGAATTCTTCAACCGGT |
| | TCCTGATCCTAATTCCCCCTCTATTCAGCATATATCACAAACGTACAATATTTCAG |
| | TATCATTTAAACAGCGTTCCCGAATGTATGGTGCTACTGTCATAGTACGAGGGTC |
| | TCAGAATAACACTAGTGCTGTGAAGGAAGGAACTGCCATGCTGTTAGAACATCTT |
| | GCTGGGAGCTTAGCATCAGCTATTCCTGTGAGCACACAACTAGATATTGCAGCTC |
| | AACATCATCTCTTTATGATGGGTCGAAATGGGAGCAACATCAAACATATCATGCA |
| | GAGAACAGGTGCTCAGATCCACTTTCCTGATCCCAGTAATCCACAAAAGAAATCT |
| | ACCGTCTACCTCCAGGGCACCATTGAGTCTGTCTGTCTTGCAAGGCAATATCTCA |
| | TGGGTTGTCTTCCTCTTGTGTTGATGTTTGATATGAAGGAAGAAATTGAAGTAGA |
| | TCCACAATTCATTGCGCAGTTGATGGAACAGCTTGATGTCTTCATCAGTATTAAA |
| | CCAAAGCCCAAACAGCCAAGCAAGTCTGTGATTGTGAAAAGTGTTGAGCGAAAT |
| | GCCTTAAATATGTATGAAGCAAGGAAATGTCTCCTCGGACTTGAAAGCAGTGGG |
| | GTTACCATAGCAACCAGTCCATCCCCAGCATCCTGCCCTGCCGGCCTGGCATGTC |
| | CCAGCCTGGATATCTTAGCTTCAGCAGGCCTTGGACTCACTGGACTAGGTCTTTT |
| | GGGACCCACCACCTTATCTCTGAACACTTCAACAACCCCAAACTCACTCTTGAAT |
| | GCTCTTAATAGCTCAGTCAGTCCTTTGCAAAGTCCAAGTTCTGGTACACCCAGCC |
| | CCACATTATGGGCACCCCCACTTGCTAATACTTCAAGTGCCACAGGTTTTTCTGCT |
| | ATACCACACCTTATGATTCCATCTACTGCCCAAGCCACATTAACTAATATTTTGTT |
| | GTCTGGAGTGCCCACCTATGGGCACACAGCTCCATCTCCCCCTCCTGGCTTGACT |
| | CCTGTTGATGTCCATATCAACAGTATGCAGACCGAAGGCAAAAAAATCTCTGCTG |
| | CTTTAAATGGACATGCACAGTCTCCAGATATAAAATATGGTGCAATATCCACTTC |
| | ATCACTTGGAGAAAAAGTCCTGAGTGCAAATCACGGGGATCCGTCCATCCAGAC |
| | AAGTGGGTCTGAGCAGACATCTCCCAAATCAAGCCCCACTGAAGGTTGTAATGA |
| | TGCTTTTGTTGAAGTAGGCATGCCTCGAAGTCCTTCCCATTCTGGGAATGCTGGT |
| | GACTTGAAACAGATGATGTGTCCCTCCAAGGTTTCCTGTCCCAAAAGGCAGACA |
| | GTGGAACTATTGCAAGGCACGAAAAACTCACACTTACACAGCACTGACAGGTTG |
| | CTCTCAGACCCTGAACTGAGTGCTACCGAAAGCCCTTTGGCTGACAAGAAGGCTC |
| | CAGGGAGTGAGCGCGCTGCAGAGAGGGCAGCAGCTGCCCAGCAAAACTCCGAA |
| | AGGGCCCACCTTGCTCCACGGTCATCATATGTCAACATGCAGGCATTTGACTATG |
| | AACAGAAGAAGCTATTAGCCACCAAAGCTATGTTAAAGAAACCAGTGGTGACGG |
| | AGGTCAGAACGCCCACAAATACCTGGAGTGGCCTGGGTTTTTCTAAATCCATGCC |
| | AGCTGAAACTATCAAGGAGTTGAGAAGGGCCAATCATGTGTCCTATAAGCCCAC |
| | AATGACAACCACTTATGAGGGCTCATCCATGTCCCTTTCACGGTCCAACAGTCGT |
| | GAGCACTTGGGAGGTGGAAGCGAATCTGATAACTGGAGAGACCGAAATGGAATT |
| | GGACCTGGAAGTCATAGTGAATTTGCAGCTTCTATTGGCAGCCCTAAGCGTAAAC |
| | AAAACAAATCAACGGAACACTATCTCAGCAGTAGCAATTACATGGACTGCATTT |
| | CCTCGCTGACAGGAAGCAATGGCTGTAACTTAAATAGCTCTTTCAAAGGTTCTGA |
| | CCTCCCTGAGCTCTTCAGCAAACTGGGCCTGGGCAAATACACAGATGTTTTCCAG |
| | CAACAAGAGATCGATCTTCAGACATTCCTCACTCTCACAGATCAGGATCTGAAGG |
| | AGCTGGGAATAACTACTTTTGGTGCCAGGAGGAAAATGCTGCTTGCAATTTCAGA |
| | ACTAAATAAAAACCGAAGAAAGCTTTTTGAATCGCCAAATGCACGCACCTCTTTC |
| | CTGGAAGGTGGAGCGAGTGGAAGGCTACCCCGTCAGTATCACTCAGACATTGCT |
| | AGTGTCAGTGGCCGCTGGTAG |
| FGFR2-CASP7 | >ATGGTCAGCTGGGGTCGTTTCATCTGCCTGGTCGTGGTCACCATGGCAACCTTGT |
| (3213 base pairs) | CCCTGGCCCGGCCCTCCTTCAGTTTAGTTGAGGATACCACATTAGAGCCAGAAGA |
| (SEQ ID NO: 37) | GCCACCAACCAAATACCAAATCTCTCAACCAGAAGTGTACGTGGCTGCGCCAGG |
| | GGAGTCGCTAGAGGTGCGCTGCCTGTTGAAAGATGCCGCCGTGATCAGTTGGACT |
| | AAGGATGGGGTGCACTTGGGGCCCAACAATAGGACAGTGCTTATTGGGGAGTAC |
| | TTGCAGATAAAGGGCGCCACGCCTAGAGACTCCGGCCTCTATGCTTGTACTGCCA |
| | GTAGGACTGTAGACAGTGAAACTTGGTACTTCATGGTGAATGTCACAGATGCCAT |
| | CTCATCCGGAGATGATGAGGATGACACCGATGGTGCGGAAGATTTTGTCAGTGA |
| | GAACAGTAACAACAAGAGAGCACCATACTGGACCAACACAGAAAAGATGGAAA |
| | AGCGGCTCCATGCTGTGCCTGCGGCCAACACTGTCAAGTTTCGCTGCCCAGCCGG |
| | GGGGAACCCAATGCCAACCATGCGGTGGCTGAAAAACGGGAAGGAGTTTAAGCA |
| | GGAGCATCGCATTGGAGGCTACAAGGTACGAAACCAGCACTGGAGCCTCATTAT |
| | GGAAAGTGTGGTCCCATCTGACAAGGGAAATTATACCTGTGTAGTGGAGAATGA |
| | ATACGGGTCCATCAATCACACGTACCACCTGGATGTTGTGGAGCGATCGCCTCAC |
| | CGGCCCATCCTCCAAGCCGGACTGCCGGCAAATGCCTCCACAGTGGTCGGAGGA |
| | GACGTAGAGTTTGTCTGCAAGGTTTACAGTGATGCCCAGCCCCACATCCAGTGGA |
| | TCAAGCACGTGGAAAAGAACGGCAGTAAATACGGGCCCGACGGGCTGCCCTACC |
| | TCAAGGTTCTCAAGGCCGCCGGTGTTAACACCACGGACAAAGAGATTGAGGTTC |
| | TCTATATTCGGAATGTAACTTTTGAGGACGCTGGGGAATATACGTGCTTGGCGGG |
| | TAATTCTATTGGGATATCCTTTCACTCTGCATGGTTGACAGTTCTGCCAGCGCCTC |
| | GAAGAGAAAAGGAGATTACAGCTTCCCCAGACTACCTGGAGATAGCCATTTACT |
| | GCATAGGGGTCTTCTTAATCGCCTGTATGGTGGTAACAGTCATCCTGTGCCGAAT |
| | GAAGAACACGACCAAGAAGCCAGACTTCAGCAGCCAGCCGGCTGTGCACAAGCT |
| | GACCAAACGTATCCCCCTGCGGAGACAGGTAACAGTTTCGGCTGAGTCCAGCTCC |
| | TCCATGAACTCCAACACCCCGCTGGTGAGGATAACAACACGCCTCTCTTCAACGG |
| | CAGACACCCCCATGCTGGCAGGGGTCTCCGAGTATGAACTTCCAGAGGACCCAA |
| | AATGGGAGTTTCCAAGAGATAAGCTGACACTGGGCAAGCCCCTGGGAGAAGGTT |
| | GCTTTGGGCAAGTGGTCATGGCGGAAGCAGTGGGAATTGACAAAGACAAGCCCA |
| | AGGAGGCGGTCACCGTGGCCGTGAAGATGTTGAAAGATGATGCCACAGAGAAAG |
| | ACCTTTCTGATCTGGTGTCAGAGATGGAGATGATGAAGATGATTGGGAAACACA |
| | AGAATATCATAAATCTTCTTGGAGCCTGCACACAGGATGGGCCTCTCTATGTCAT |
| | AGTTGAGTATGCCTCTAAAGGCAACCTCCGAGAATACCTCCGAGCCCGGAGGCC |
| | ACCCGGGATGGAGTACTCCTATGACATTAACCGTGTTCCTGAGGAGCAGATGACC |
| | TTCAAGGACTTGGTGTCATGCACCTACCAGCTGGCCAGAGGCATGGAGTACTTGG |
| | CTTCCCAAAAATGTATTCATCGAGATTTAGCAGCCAGAAATGTTTTGGTAACAGA |
| | AAACAATGTGATGAAAATAGCAGACTTTGGACTCGCCAGAGATATCAACAATAT |
| | AGACTATTACAAAAAGACCACCAATGGGCGGCTTCCAGTCAAGTGGATGGCTCC |
| | AGAAGCCCTGTTTGATAGAGTATACACTCATCAGAGTGATGTCTGGTCCTTCGGG |
| | GTGTTAATGTGGGAGATCTTCACTTTAGGGGGCTCGCCCTACCCAGGGATTCCCG |
| | TGGAGGAACTTTTTAAGCTGCTGAAGGAAGGACACAGAATGGATAAGCCAGCCA |
| | ACTGCACCAACGAACTGTACATGATGATGAGGGACTGTTGGCATGCAGTGCCCTC |
| | CCAGAGACCAACGTTCAAGCAGTTGGTAGAAGACTTGGATCGAATTCTCACTCTC |
| | ACAACCAATGAGATGGCAGATGATCAGGGCTGTATTGAAGAGCAGGGGGTTGAG |
| | GATTCAGCAAATGAAGATTCAGTGGATGCTAAGCCAGACCGGTCCTCGTTTGTAC |
| | CGTCCCTCTTCAGTAAGAAGAAGAAAAATGTCACCATGCGATCCATCAAGACCA |
| | CCCGGGACCGAGTGCCTACATATCAGTACAACATGAATTTTGAAAAGCTGGGCA |
| | AATGCATCATAATAAACAACAAGAACTTTGATAAAGTGACAGGTATGGGCGTTC |
| | GAAACGGAACAGACAAAGATGCCGAGGCGCTCTTCAAGTGCTTCCGAAGCCTGG |
| | GTTTTGACGTGATTGTCTATAATGACTGCTCTTGTGCCAAGATGCAAGATCTGCTT |
| | AAAAAAGCTTCTGAAGAGGACCATACAAATGCCGCCTGCTTCGCCTGCATCCTCT |
| | TAAGCCATGGAGAAGAAAATGTAATTTATGGGAAAGATGGTGTCACACCAATAA |
| | AGGATTTGACAGCCCACTTTAGGGGGGATAGATGCAAAACCCTTTTAGAGAAAC |
| | CCAAACTCTTCTTCATTCAGGCTTGCCGAGGGACCGAGCTTGATGATGGCATCCA |
| | GGCCGACTCGGGGCCCATCAATGACACAGATGCTAATCCTCGATACAAGATCCC |
| | AGTGGAAGCTGACTTCCTCTTCGCCTATTCCACGGTTCCAGGCTATTACTCGTGG |
| | AGGAGCCCAGGAAGAGGCTCCTGGTTTGTGCAAGCCCTCTGCTCCATCCTGGAGG |
| | AGCACGGAAAAGACCTGGAAATCATGCAGATCCTCACCAGGGTGAATGACAGAG |
| | TTGCCAGGCACTTTGAGTCTCAGTCTGATGACCCACACTTCCATGAGAAGAAGCA |
| | GATCCCCTGTGTGGTCTCCATGCTCACCAAGGAACTCTACTTCAGTCAATAG |

FGFR genetic alterations include FGFR single nucleotide polymorphism (SNP). “FGFR single nucleotide polymorphism” (SNP) refers to a FGFR2 or FGFR3 gene in which a single nucleotide differs among individuals. In certain embodiments, the FGFR2 or FGFR3 genetic alteration is an FGFR3 gene mutation. In particular, “FGFR single nucleotide polymorphism” (SNP) refers to a FGFR3 gene in which a single nucleotide differs among individuals. The presence of one or more of the following FGFR SNPs in a biological sample from a patient can be determined by methods known to those of ordinary skill in the art or methods disclosed in WO 2016/048833, FGFR3 R248C, FGFR3 S249C, FGFR3 G370C, FGFR3 Y373C, or any combination thereof. The sequences of the FGFR SNPs are provided in Table B.

TABLE B

| FGFR3 mutant | Sequence |
|---|---|
| FGFR3 R248C | TCGGACCGCGGCAACTACACCTGCGTCGTGGAGAACAAGTTTGGCAGCA |
| | TCCGGCAGACGTACACGCTGGACGTGCTGGAG(T)GCTCCCCGCACCGGC |
| | CCATCCTGCAGGCGGGGCTGCCGGCCAACCAGACGGCGGTGCTGGGCAG |
| | CGACGTGGAGTTCCACTGCAAGGTGTACAGTGACGCACAGCCCCACATC |
| | CAGTGGCTCAAGCACGTGGAGGTGAATGGCAGCAAGGTGGGCCCGGACG |
| | GCACACCCTACGTTACCGTGCTCA |
| | (SEQ ID NO: 1) |
| FGFR3 S249C | GACCGCGGCAACTACACCTGCGTCGTGGAGAACAAGTTTGGCAGCATCC |
| | GGCAGACGTACACGCTGGACGTGCTGGGTGAGGGCCCTGGGGCGGCGCG |
| | GGGGTGGGGGCGGCAGTGGCGGTGGTGGTGAGGGAGGGGGTGGCCCCT |
| | GAGCGTCATCTGCCCCCACAGAGCGCT(G)CCCGCACCGGCCCATCCTGCA |
| | GGCGGGGCTGCCGGCCAACCAGACGGCGGTGCTGGGCAGCGACGTGGAG |
| | TTCCACTGCAAGGTGTACAGTGACGCACAGCCCCACATCCAGTGGCTCAA |
| | GCACGTGGAGGTGAATGGCAGCAAGGTGGGCCCGGACGGCACACCCTAC |
| | GTTACCGTGCTCAAGGTGGGCCACCGTGTGCACGT |
| | (SEQ ID NO: 2) |
| FGFR3 G370C | GCGGGCAATTCTATTGGGTTTTCTCATCACTCTGCGTGGCTGGTGGTGCT |
| | GCCAGCCGAGGAGGAGCTGGTGGAGGCTGACGAGGCG(T)GCAGTGTGTA |
| | TGCAGGCATCCTCAGCTACGGGGTGGGCTTCTTCCTGTTCATCCTGGTGG |
| | TGGCGGCTGTGACGCTCTGCCGCCTGCGCAGCCCCCCCAAGAAAGGCCT |
| | GGGCTCCCCCACCGTGCACAAGATCTCCCGCTTCCCG |
| | (SEQ ID NO: 3) |
| FGFR3 Y373C* | CTAGAGGTTCTCTCCTTGCACAACGTCACCTTTGAGGACGCCGGGGAGTA |
| | CACCTGCCTGGCGGGCAATTCTATTGGGTTTTCTCATCACTCTGCGTGGCT |
| | GGTGGTGCTGCCAGCCGAGGAGGAGCTGGTGGAGGCTGACGAGGCGGGC |
| | AGTGTGT(G)TGCAGGCATCCTCAGCTACGGGGGGGCTTCTTCCTGTTCA |
| | TCCTGGTGGTGGCGGCTGTGACGCTCTGCCGCCTGCGCAGCCCCCCCAAG |
| | AAAGGCCTGGGCTCCCCCACCGTGCACAAGATCTCCCGCTTCCCGCTCAA |
| | GC |
| | (SEQ ID NO: 4) |

Sequences correspond to nucleotides 920-1510 of FGFR3 (Genebank ID # NM_000142.4).
Nucleotides in bold underline represent the SNP.
*Sometimes mistakenly referred to as Y375C in the literature.

In certain embodiments, the methods of or uses for treating a urothelial carcinoma as described herein comprise, consist of, or consist essentially of administering the drug delivery system as described herein to a patient that has been diagnosed with a urothelial carcinoma as described herein and harbors at least one FGFR2 genetic alteration and/or FGFR3 genetic alteration (i.e., one or more FGFR2 genetic alteration, one or more FGFR3 genetic alteration, or a combination thereof). In certain embodiments, the FGFR2 genetic alteration and/or FGFR3 genetic alteration is an FGFR3 gene mutation, FGFR2 gene fusion, or FGFR3 gene fusion. In some embodiments, the FGFR3 gene mutation is R248C, S249C, G370C, Y373C, or any combination thereof. In still further embodiments, the FGFR2 or FGFR3 gene fusion is FGFR3-TACC3, FGFR3-BAIAP2L1, FGFR2-BICC1, FGFR2-CASP7, or any combination thereof.

Also described herein are methods or uses of treating a urothelial carcinoma as described herein comprising, consisting of, or consisting essential of: (a) evaluating a biological sample from a patient with a urothelial carcinoma as described herein for the presence of one or more FGFR gene alterations, in particular one or more FGFR2 or FGFR3 gene alterations; and (b) administering a drug delivery system as described herein to the patient if one or more FGFR gene alterations, in particular one or more FGFR2 or FGFR3 gene alterations, is present in the sample.

The following methods for evaluating a biological sample for the presence of one or more FGFR genetic alterations apply equally to any of the above disclosed methods of treatment and uses.

Suitable methods for evaluating a biological sample for the presence of one or more FGFR genetic alterations are described herein and in WO 2016/048833 and U.S. patent application Ser. No. 16/723,975, which are incorporated herein in their entireties. For example, and without intent to be limiting, evaluating a biological sample for the presence of one or more FGFR genetic alterations can comprise any combination of the following steps: isolating RNA from the biological sample; synthesizing cDNA from the RNA; and amplifying the cDNA (preamplified or non-preamplified). In some embodiments, evaluating a biological sample for the presence of one or more FGFR genetic alterations can comprise: amplifying cDNA from the patient with a pair of primers that bind to and amplify one or more FGFR genetic alterations; and determining whether the one or more FGFR genetic alterations are present in the sample. In some aspects, the cDNA can be pre-amplified. In some aspects, the evaluating step can comprise isolating RNA from the sample, synthesizing cDNA from the isolated RNA, and pre-amplifying the cDNA.

Suitable primer pairs for performing an amplification step include, but are not limited to, those disclosed in WO 2016/048833, as exemplified below in Table C:

TABLE C

| Target | Forward Primer | Reverse Primer 5′-3′ |
|---|---|---|
| FGFR3-TACC3 | GACCTGGACCGTGTCCTTACC | CTTCCCCAGTTCCAGGTTCTT |
| V1 | (SEQ ID NO: 5) | (SEQ ID NO: 6) |
| FGFR3-TACC3 | AGGACCTGGACCGTGTCCTT | TATAGGTCCGGTGGACAGGG |
| V3 | (SEQ ID NO: 7) | (SEQ ID NO: 8) |
| FGFR3- | CTGGACCGTGTCCTTACCGT | GCAGCCCAGGATTGAACTGT |
| BAIAP2L1 | (SEQ ID NO: 9) | (SEQ ID NO: 10) |
| FGFR2-BICC1 | TGGATCGAATTCTCACTCTCA | GCCAAGCAATCTGCGTATTTG |
| | CA | (SEQ ID NO: 12) |
| | (SEQ ID NO: 11) | |
| FGFR2-CASP7 | GCTCTTCAATACAGCCCTGAT | ACTTGGATCGAATTCTCACTCT |
| | CA | CA |
| | (SEQ ID NO: 13) | (SEQ ID NO: 14) |
| FGFR2-CCDC6 | TGGATCGAATTCTCACTCTCA | GCAAAGCCTGAATTTTCTTGA |
| | CA | ATAA |
| | (SEQ ID NO: 15) | (SEQ ID NO: 16) |
| FGFR3 R248C | GCATCCGGCAGACGTACA | CCCCGCCTGCAGGAT |
| | (SEQ ID NO: 17) | (SEQ ID NO: 18) |
| FGFR3 S249C | GCATCCGGCAGACGTACA | CCCCGCCTGCAGGAT |
| | (SEQ ID NO: 19) | (SEQ ID NO: 20) |
| FGFR3 G370C | AGGAGCTGGTGGAGGCTGA | CCGTAGCTGAGGATGCCTG |
| | (SEQ ID NO: 21) | (SEQ ID NO: 22) |
| FGFR3 Y373C | CTGGTGGAGGCTGACGAG | AGCCCACCCCGTAGCT |
| | (SEQ ID NO: 23) | (SEQ ID NO: 24) |
| FGFR3 R248C | GTCGTGGAGAACAAGTTTGGC | GTCTGGTTGGCCGGCAG |
| | (SEQ ID NO: 25) | (SEQ ID NO: 26) |
| FGFR3 S249C | GTCGTGGAGAACAAGTTTGGC | GTCTGGTTGGCCGGCAG |
| | (SEQ ID NO: 27) | (SEQ ID NO: 28) |
| FGFR3 G370C | AGGAGCTGGTGGAGGCTGA | CCGTAGCTGAGGATGCCTG |
| | (SEQ ID NO: 29) | (SEQ ID NO: 30) |
| FGFR3 Y373C | GACGAGGCGGGCAGTG | GAAGAAGCCCACCCCGTAG |
| | (SEQ ID NO: 31) | (SEQ ID NO: 32) |

The presence of one or more FGFR genetic alterations can be evaluated at any suitable time point including upon diagnosis, following tumor resection, following first-line therapy, during clinical treatment, or any combination thereof.

The methods and uses can further comprise evaluating the presence of one or more FGFR genetic alterations in the biological sample before the administering step.

The diagnostic tests and screens are typically conducted on a biological sample selected from blood, lymph fluid, bone marrow, a solid tumor sample, or any combination thereof. In certain embodiments, the biological sample is a solid tumor sample. In certain embodiments, the biological sample is a blood sample.

Methods of identification and analysis of genetic alterations and up-regulation of proteins are known to a person skilled in the art Screening methods could include, but are not limited to, standard methods such as reverse-transcriptase polymerase chain reaction (RT PCR) or in-situ hybridization such as fluorescence in situ hybridization (FISH).

Identification of an individual carrying a genetic alteration in FGFR, in particular an FGFR genetic alteration as described herein, may mean that the patient would be particularly suitable for treatment with erdafitinib. Tumors may preferentially be screened for presence of a FGFR variant prior to treatment. The screening process will typically involve direct sequencing, oligonucleotide microarray analysis, or a mutant specific antibody. In addition, diagnosis of tumor with such genetic alteration could be performed using techniques known to a person skilled in the art and as described herein such as RT-PCR and FISH.

In addition, genetic alterations of, for example FGFR, can be identified by direct sequencing of, for example, tumor biopsies using PCR and methods to sequence PCR products directly as hereinbefore described. The skilled artisan will recognize that all such well-known techniques for detection of the over expression, activation or mutations of the aforementioned proteins could be applicable in the present case.

In screening by RT-PCR, the level of mRNA in the tumor is assessed by creating a cDNA copy of the mRNA followed by amplification of the cDNA by PCR. Methods of PCR amplification, the selection of primers, and conditions for amplification, are known to a person skilled in the art. Nucleic acid manipulations and PCR are carried out by standard methods, as described for example in Ausubel, F. M. et al., eds. (2004) Current Protocols in Molecular Biology, John Wiley & Sons Inc., or Innis, M. A. et al., eds. (1990) PCR Protocols: a guide to methods and applications. Academic Press, San Diego. Reactions and manipulations involving nucleic acid techniques are also described in Sambrook et al., (2001), 3rd Ed, Molecular Cloning: A Laboratory Manual, Cold Spring Harbor Laboratory Press. Alternatively, a commercially available kit for RT-PCR (for example Roche Molecular Biochemicals) may be used, or methodology as set forth in U.S. Pat. Nos. 4,666,828; 4,683,202; 4,801,531, 5,192,659, 5,272,057, 5,882,864, and 6,218,529 and incorporated herein by reference. An example of an in-situ hybridization technique for assessing mRNA expression would be fluorescence in-situ hybridization (FISH) (see Angerer (1987) Meth. Enzymol., 152: 649).

Generally, in situ hybridization comprises the following major steps: (1) fixation of tissue to be analyzed; (2) prehybridization treatment of the sample to increase accessibility of target nucleic acid, and to reduce nonspecific binding. (3) hybridization of the mixture of nucleic acids to the nucleic acid in the biological structure or tissue; (4) post-hybridization washes to remove nucleic acid fragments not bound in the hybridization, and (5) detection of the hybridized nucleic acid fragments. The probes used in such applications are typically labelled, for example, with radioisotopes or fluorescent reporters. Preferred probes are sufficiently long, for example, from about 50, 100, or 200 nucleotides to about 1000 or more nucleotides, to enable specific hybridization with the target nucleic acid(s) under stringent conditions. Standard methods for carrying out FISH are described in Ausubel, F. M. et al., eds. (2004) Current Protocols in Molecular Biology, John Wiley & Sons Inc and Fluorescence In Situ Hybridization: Technical Overview by John M. S. Bartlett in Molecular Diagnosis of Cancer, Methods and Protocols, 2nd ed., ISBN: 1-59259-760-2; March 2004, pps. 077-088; Series. Methods in Molecular Medicine.

Methods for gene expression profiling are described by (DePrimo et al. (2003), BMC Cancer, 3:3). Briefly, the protocol is as follows: double-stranded cDNA is synthesized from total RNA Using a (dT)24 oligomer (SEQ ID NO: 38: tttttttttt tttttttttt tttt) for priming first-strand cDNA synthesis, followed by second strand cDNA synthesis with random hexamer primers. The double-stranded cDNA is used as a template for in vitro transcription of cRNA using biotinylated ribonucleotides. cRNA is chemically fragmented according to protocols described by Affymetrix (Santa Clara, CA, USA), and then hybridized overnight on Human Genome Arrays.

Alternatively, the protein products expressed from the mRNAs may be assayed by immunohistochemistry of tumor samples, solid phase immunoassay with microtitre plates, Western blotting, 2-dimensional SDS-polyacrylamide gel electrophoresis, ELISA, flow cytometry and other methods known in the art for detection of specific proteins. Detection methods would include the use of site-specific antibodies. The skilled person will recognize that all such well-known techniques for detection of upregulation of FGFR or detection of FGFR variants or mutants could be applicable in the present case.

Abnormal levels of proteins such as FGFR can be measured using standard enzyme assays, for example, those assays described herein. Activation or overexpression could also be detected in a tissue sample, for example, a tumor tissue, by measuring the tyrosine kinase activity with an assay such as that from Chemicon International. The tyrosine kinase of interest would be immunoprecipitated from the sample lysate and its activity measured.

Alternative methods for the measurement of the over expression or activation of FGFR including the isoforms thereof, include the measurement of microvessel density. This can for example be measured using methods described by Orre and Rogers (Int J Cancer (1999), 84(2) 101-8). Assay methods also include the use of markers.

Therefore, all of these techniques could also be used to identify tumors particularly suitable for treatment with the drug delivery systems of the invention.

According to certain embodiments, FGFR2 and/or FGFR3 genetic alterations can be identified using commercially available kits including, but not limiting to, a QIAGEN Therascreen® FGFR RGQ RT-PCR kit.

In certain embodiments, a method of administering a drug to a patient includes inserting a drug delivery system as described herein into a patient and permitting the drug to be released from the system. For example, the system may include any features, or combinations of features, described herein. In certain embodiments, a release profile of the drug is substantially independent of pH over a pH range of 5 to 7.

In certain embodiments, permitting the drug to be released from the system includes permitting water to be imbibed through the water permeable wall bounding the drug reservoir lumen to contact and solubilize the drug formulation and generate an osmotic pressure within the drug reservoir lumen; and then permitting the solubilized drug to be released from the system through an aperture in the system, driven by the osmotic pressure. That is, in certain embodiments, elution of drug from the system occurs following dissolution of the drug within the system. Bodily fluid, e.g., urine, enters the system, contacts the drug and solubilizes the drug, and thereafter the dissolved drug is pumped out of the system by osmotic pumping through one or more apertures in fluid communication with the drug reservoir lumen.

In certain embodiments, the inserting comprises deploying the system through the patient's urethra and into the patient's urinary bladder. The system may release drug for several days, weeks, months, or more after the insertion procedure has ended. In one embodiment, deploying the drug delivery system in the patient includes inserting the system into a body cavity or lumen of the patient via a deployment instrument. For example, the system may be deployed through a deployment instrument, such as a catheter or cystoscope, positioned in a natural lumen of the body, such as the urethra, or into a body cavity, such as the bladder. The deployment instrument typically is removed from the body lumen while the drug delivery system remains in the bladder or other body cavity for a prescribed treatment period.

In one example, the system is deployed by passing the drug delivery system through a deployment instrument and releasing the system from the deployment instrument into the bladder. In embodiments, the system assumes a retention shape, such as an expanded or higher profile shape, once the system emerges from the deployment instrument into the bladder. The deployment instrument may be a commercially available system or a system specially adapted for the present drug delivery systems. In one embodiment, deploying the drug delivery system in the patient includes (i) elastically deforming the system into the relatively straightened shape; (ii) inserting the system through the patient's urethra, through the lumen of an insertion catheter, for example driven by a stylet; and (iii) releasing the system from the insertion catheter into the patient's bladder such that it assumes a coiled retention shape.

Once deployed in vivo, the system subsequently releases the drug (e.g., erdafitinib) for the treatment of one or more conditions or diseases, locally to tissues at the deployment site. The release is controlled to release the drug in an effective amount over an extended period. In certain embodiments, the system resides in the bladder releasing the drug over a predetermined period, such as two weeks, three weeks, four weeks, six weeks, two months, three months, or more.

The deployed system releases a desired quantity of drug over a desired, predetermined period. In embodiments, the system can deliver the desired dose of drug over an extended period, such as from 2 days to 90 days (e.g., 3, 5, 7, 10, 14, 20, 21, 25, 28, 30, 40, 45, 50, 60, 70, 80 or 90 days), from 1 month to 6 months (e.g., 6 weeks, 1 month, 2 months, 3 months, 4 months, or 5 months) or more. The rate of delivery and dosage of the drug can be selected depending upon the drug being delivered and the disease or condition being treated. In one embodiment, a rate of release of the drug from the drug delivery system is zero order over at least 36 hours. In one embodiment, a rate of the release of the drug from the drug delivery system is essentially zero order over at least 7 days, two weeks, three weeks, four weeks, a month, two months, three months or more.

Subsequently, the system may be retrieved from the bladder through the urethra using a cystoscope or catheter. If needed, a new drug-loaded system may subsequently be inserted, during a same office procedure as the retrieval, or at a later time.

The present disclosure may be further understood with reference to the following non-limiting examples.

### Enumerated Embodiments

1. A drug delivery system comprising:
an elongated body configured for intravesical insertion into a patient; and
a drug formulation located within the elongated body, the drug formulation comprising erdafitinib (N-(3,5-dimethoxyphenyl)-N′-(1-methylethyl)-N-[3-(1-methyl-1H-pyrazol-4-yl)quinoxalin-6-yl]ethane-1,2-diamine) or a pharmaceutically acceptable salt thereof,
wherein the drug delivery system is configured to release the erdafitinib from one or more openings in the elongated body.

2. The drug delivery system of embodiment 1, wherein the drug formulation comprises a salt form of the erdafitinib.

3. The drug delivery system of embodiment 2, wherein the salt form comprises an L-lactate salt of erdafitinib.

4. The drug delivery system of embodiment 1, wherein erdafitinib is present as a mono L-lactate salt.

5. The drug delivery system of any one of embodiments 1 to 4, wherein the elongated body comprises a biocompatible elastomer.

6. The drug delivery system of embodiment 5, wherein the biocompatible elastomer comprises silicone or a thermoplastic polyurethane.

7. The drug delivery system of any one of embodiments 1 to 6, wherein at least one of the one or more openings in the elongated body is located in a sidewall of the elongated body.

8. The drug delivery system of any one of embodiments 1 to 7, wherein at least one of the one or more openings in the elongated body is located at a first end and/or at an opposing second end of the elongated body.

9. The drug delivery system of any one of embodiments 1 to 6, having a single opening, which is located in a sidewall of the elongated body at position between a first end and an opposing second end of the elongated body.

10. The drug delivery system of any one of embodiments 1 to 9, wherein the drug formulation comprises at least one pharmaceutical excipient.

11. The drug delivery system of embodiment 10, wherein the at least one pharmaceutical excipient comprises or is selected from a solubilizer, a binder, a diluent (filler), a wetting agent, a disintegrant, a glidant, a lubricant, a formaldehyde scavenger, an osmotic agent, or any combination thereof.

12. The drug delivery system of embodiment 10, wherein the at least one pharmaceutical excipient comprises or is selected from a binder, a diluent (filler), a glidant, a lubricant, or any combination thereof.

13. The drug delivery system of any one of embodiments 1 to 12, wherein the erdafitinib is present in the drug formulation in a concentration of from 60 wt % to 80 wt %.

14. The drug delivery system of embodiment 13, wherein the erdafitinib is present in the drug formulation in a concentration of 70 wt %.

15. The drug delivery system of any one of embodiments 1 to 14, wherein the drug formulation is in the form of a plurality of mini-tablets.

16. The drug delivery system of any one of embodiments 1 to 15, wherein the drug delivery system is configured to release the erdafitinib by osmotic pressure through one or more openings in the elongated body.

17. The drug delivery system of any one of embodiments 1 to 16, wherein the elongated body comprises an annular wall structure defining a drug reservoir lumen in which the drug formulation is disposed.

18. The drug delivery system of embodiment 17, wherein the one or more openings in the elongated body comprise a single aperture in the annular wall structure, and the elongated body is configured to release the erdafitinib through the aperture and/or through microchannels transiently formed at one or both end regions of the annular wall structure.

19. An intravesical drug delivery system comprising:
an elongated body configured for intravesical insertion into a patient; and
a drug formulation disposed in the elongated body, the drug formulation comprising an L-lactate salt of erdafitinib (N-(3,5-dimethoxyphenyl)-N′-(1-methylethyl)-N-[3-(1-methyl-1H-pyrazol-4-yl)quinoxalin-6-yl]ethane-1,2-diamine),
wherein the drug delivery system is configured to release the erdafitinib from one or more openings in the elongated body, driven by osmotic pressure.

20. The system of any one of embodiments 1 to 19, wherein the system is configured to release the erdafitinib at an average rate of 1 mg/day to 10 ng/day.

21. The system of any one of embodiments 1 to 19, wherein the system is configured to release the erdafitinib at an average rate of 1 mg/day to 6 mg/day.

22. The system of any one of embodiments 1 to 19, wherein the system is configured to release the erdafitinib at an average rate of 2 mg/day to 4 mg/day.

23. The system of any one of embodiments 1 to 19, wherein the system is configured to release the erdafitinib at an average rate of 4 mg/day.

24. The system of any one of embodiments 1 to 19, wherein the system is configured to release the erdafitinib at an average rate of 2 mg/day.

25. The system of any one of embodiments 1 to 24, wherein the system comprises 500 mg of the erdafitinib (free base equivalent).

26. The system of any one of embodiments 1 to 25, wherein the system is elastically deformable between a relatively straightened deployment shape suited for insertion through the urethra of a patient and into the patient's bladder and a retention shape suited to retain the system within the bladder.

27. The system of any one of embodiments 1 to 26, wherein the system is elastically deformable and comprises a tube having two opposing free ends, which are directed away from one another when the system is in a low-profile deployment shape and which are directed toward one another when the system is in a relatively expanded retention shape.

28. The system of any one of embodiments 1 to 27, wherein the system comprises an elastically deformable elongated body having two opposing free ends which lie within the boundaries of a bi-oval-like expanded retention shape.

29. The system of any one of embodiments 1 to 28, wherein the elongated body further comprises a retention frame lumen.

30. The system of embodiment 29, further comprising a nitinol wire disposed in the retention frame lumen.

31. A method of intravesical administration of erdafitinib, comprising:
deploying a drug delivery system into the bladder of a patient; and
releasing erdafitinib from the drug delivery system by osmotic pressure.

32. A method of intravesical administration of erdafitinib, comprising.
deploying the system of any one of embodiments 1 to 30 into the bladder of a patient; and
releasing the erdafitinib from the system.

33. The method of embodiment 31 or 32, wherein the releasing the erdafitinib from the system comprises releasing the erdafitinib from a drug reservoir lumen through the one or more openings driven by osmotic pressure in the drug reservoir lumen.

34. The method of any one of embodiments 31 to 33, wherein the system is elastically deformed into a low-profile deployment shape and inserted through the urethra and into the patient's bladder, and then assumes a relatively expanded retention shape within the bladder.

35. The method of any one of embodiments 31 to 34, wherein erdafitinib is released into the bladder at an average rate of 1 mg/day to 10 mg/day over a period ranging from 7 days to 90 days.

36. A method of treating non-muscle invasive bladder cancer (NMIBC) or muscle invasive bladder cancer (MIBC) in a cancer patient, comprising:
inserting a drug delivery system into the bladder of the patient; and
locally delivering into the bladder of the patient a therapeutically effective amount of erdafitinib from the drug delivery system, driven by osmotic pressure.

37. A method of treating non-muscle invasive bladder cancer (NMIBC) or muscle invasive bladder cancer (MIBC) in a cancer patient, comprising:
inserting into the bladder of the patient the system of any one of embodiments 1 to 30, and
locally delivering a therapeutically effective amount of erdafitinib from the system into the bladder of the patient.

38. The method of embodiment 36 or 37, wherein the locally delivering erdafitinib comprises releasing the erdafitinib from the system at a release rate from about 1 mg/day to about 6 mg/day, such as 2 to 4 mg/day.

39. The method of any one of embodiments 36 to 38, wherein the system is maintained in the patient's bladder for up to 90 days, and then, optionally, replaced with another erdafitinib-releasing system.

40. A method of treating (i) recurrent, non-muscle-invasive or muscle-invasive urothelial carcinoma of the bladder, (ii) high- or intermediate-risk papillary urothelial carcinoma of the bladder, or (iii) muscle-invasive urothelial carcinoma of the bladder staged cT2-T3a in a cancer patient, comprising:
inserting a drug delivery system into the bladder of the patient; and
locally delivering a therapeutically effective amount of erdafitinib from the drug delivery system into the bladder of the patient, in particular driven by osmotic pressure.

41. A method of treating (i) recurrent, non-muscle-invasive or muscle-invasive urothelial carcinoma of the bladder, (ii) high- or intermediate-risk papillary urothelial carcinoma of the bladder, or (iii) muscle-invasive urothelial carcinoma of the bladder staged cT2-T3a in a cancer patient, comprising:
inserting into the bladder of the patient the system of any one of embodiments 1 to 30; and
locally delivering a therapeutically effective amount of erdafitinib from the system into the bladder of the patient.

42. The method of embodiment 40 or 41, wherein the patient undergoes transurethral resection of bladder tumor (TURBT) to reduce the total tumor(s) size to less than or equal to 3 cm, prior to the locally delivering the erdafitinib into the bladder.

43. The method of any one of embodiments 40 to 42, wherein the locally delivering of erdafitinib comprises releasing the erdafitinib from the system at a release rate from about 1 mg/day to about 6 mg/day, such as 2 to 4 mg/day.

44. The method of any one of embodiments 40 to 43, wherein the system is maintained in the patient's bladder for up to 90 days, and then, optionally, replaced with another erdafitinib-releasing system.

45. A method of treating a Bacillus Calmette-Guerin (BCG) experienced patient having recurrent high-grade Ta/T1 urothelial carcinoma of the bladder within 18 months of completion of prior BCG therapy, comprising.
inserting a drug delivery system into the bladder of the patient; and
locally delivering a therapeutically effective amount of erdafitinib from the drug delivery system into the bladder of the patient.

46. A method of treating a Bacillus Calmette-Guerin (BCG) experienced patient having recurrent high-grade Ta/Tl urothelial carcinoma of the bladder within 18 months of completion of prior BCG therapy, comprising:
locally delivering a therapeutically effective amount of erdafitinib from the system into the bladder of the patient, in particular from a system of any one of embodiments 1 to 30 inserted into the bladder of the patient.

47. The method of embodiment 45 or 46, wherein the locally delivering of erdafitinib comprises releasing the erdafitinib from the system at a release rate from about 1 mg/day to about 6 mg/day, such as 2 to 4 mg/day.

48. The method of any one of embodiments 45 to 47, wherein the system is maintained in the patient's bladder for up to 90 days, and then, optionally, replaced with another erdafitinib-releasing system.

49. The method of any one of embodiments 36 to 48, wherein the patient harbors at least one FGFR2 genetic alteration and/or FGFR3 genetic alteration.

50. Erdafitinib lactate salt, in particular erdafitinib L-lactate.

51. A pharmaceutical composition comprising erdafitinib lactate salt, in particular erdafitinib L-lactate, and one or more excipients.

52. The pharmaceutical composition of embodiment 51, wherein the composition is in the form of a tablet, particularly a mini-tablet.

### EXAMPLES

### Example 1. Screening of Materials and API Forms for Erdafitinib Release

A number of polymeric materials were tested to determine their suitability as a material of construction of an elastic system body for release of various erdafitinib formulations. The materials included silicone and several thermoplastic polyurethane (TPUs) manufactured by Lubrizol Life Science (Bethlehem, PA). The results are set forth in the Table 1.

TABLE 1
In Vitro Permeation

| | | Erdafitinib | | Erdafitinib |
|---|---|---|---|---|
| | Erdafitinib | free base + | Erdafitinib | L-lactate |
| Material | free base | HP-β-CD | HCl salt | salt |
| Silicone (MED-4750) | X | X | X | X |
| TPU (PC-3575A) | X | X | X | X |
| TPU (AR-62A) | X | X | X | X |
| TPU (HP-60D-35) | ◯ | ◯ | ◯ | ◯ |
| TPU (AC-4075A) | X | X | X | X |
| TPU (TT-1074A) | X | Δ | X | X |
| TPU (HP-93A-100) | ◯ | ◯ | ◯ | ◯ |
| TPU (EG-80A) | Δ | ◯ | Δ | Δ |
| EVA (CoTran 9712) | Δ | X | Δ | ◯ |

In Table 1, “O” is permeable, “A” is practically impermeable, and “X” is impermeable. This information may be useful in the selection of osmotic system housing materials of construction and designs that may be used with various possible erdafitinib forms/formulations.

### Example 2. Sample Ninitablet Formulations

Table 2 illustrates selected aspects and embodiments of the minitablet formulation for use with the disclosed drug delivery systems. The blend of Table 2 is for tablets with a target tablet weight of 20 mg, and drug load of 70 wt %.

TABLE 2

| | Weighed Mass (g) | Wt % |
|---|---|---|
| Erdafitinib granulate | 3692.000 | 71.43% |
| Copovidone (e.g. Kollidon VA64) | 667.280 | 12.91% |
| Microcrystalline cellulose | 667.280 | 12.91% |
| (e.g., Avicel PH105) | | |
| colloidal silica (e.g., Aerosil 200P) | 12.920 | 0.25% |
| Magnesium stearate | 129.220 | 2.5% |
| Total | 5168.700 | 100.00% |

The erdafitinib granulate was composed of 98 wt % of erdafitinib mono-L-lactate and 2 wt % of HPMC (e.g. HPMC 290 15 mPa·s).

Preparation: All compounds were screened through a 600 μm sieve. Erdafitinib granulate, copovidone, microcrystalline cellulose, and colloidal silica were weighed and mixed for 10 minutes. The blend was screened through a 600 un sieve. Magnesium stearate was added and the blend was mixed for 5 minutes. The resulting blend was tableted.

### Example 3. Erdafitinib Metabolism and Pharmacokinetic Properties for Intravesical Delivery

Erdafitinib was determined to be sufficiently stable in freshly collected human, minipig and rat urine at 37° C. for 6 hours, indicating that drug is stable in urine between void cycles.

In vitro protein binding indicated erdafitinib to be mainly present in free form in urine. Percent erdafitinib free in rat, minipig and human urine was assessed to be 84%, 97% and 95%, respectively and concentration independent. Erdafitinib found to bind to AGP less than to albumin. Albuminuria/proteinuria had minimal effect on % free form in urine.

Erdafitinib in vitro binding to normal/tumor bladder tissues showed significant free fraction Free fraction of erdafitinib in minipig bladder tissue was 79%, 2 times higher than in rat (33%) and human (39%). In tumor tissue, free fraction was 40%.

Bladder perfusion studies in pig and rat showed good partitioning from urine into the bladder, and particularly into the urothelium. Low systemic bioavailability (˜5% in rat and 12% in minipig) after localized bladder dosing was observed.

Based on these studies, erdafitinib is stable in urine and present in high free fraction, which therefore should lead to desired exposures to bladder tumors. Erdatitinib appears to have favorable drug metabolism and pharmacokinetic properties for intravesical administration.

### Example 4: Pharmacokinetics (PK) and Pharmacodynamics (PD) of Single-Dose Intravesical Erdafitinib Administration in Rats Bearing Orthoropic Bladder Tumors

The objective of Example 4 was to compare the PK and PD effects of localized bladder versus oral administration of erdafitinib in nude rats bearing human UM-UC-1 bladder xenografts. Animals were given a single oral dose (20 mg/kg erdafitinib in 10% weight per volume (w/v) HP-β-CD solution) or 1-hour intravesical instillation (6 mg/kg erdafitinib in 10% w/v HP-β-CD solution) of erdafitinib into the bladder. Extracellular signal-regulated kinase (ERK)1/2 phosphorylation was assessed as a PD marker for FGFR kinase inhibition in tumors at various time points post administration/installation. PK analysis of tumor and plasma samples was carried out at 2, 7, 48, and 120 hours after a single 6 mg/kg intravesical administration or a 20 mg/kg oral dose of erdafitinib. Additionally, a group of nude rats bearing subcutaneous (s.c.) tumors (UM-UC-1) were given erdafitinib orally and concentrations were measured in plasma and tumor at 2, 7, 48, and 120 hours post dose.

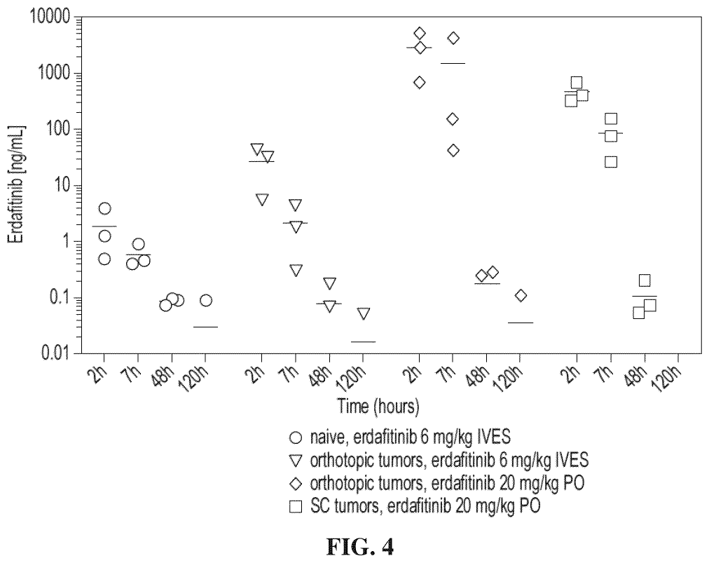

Intravesical administration resulted in mean erdafitinib exposure levels that were comparable to that of a 20 mg/kg oral dose (Table 3). Roughly 2-fold lower exposure levels were detected at 2 and 7 hours in subcutaneous (s.c.) tumors from rats dosed orally compared with orthotopic tumors from rats dosed orally, however it reflected the lower commensurate plasma exposures observed in the same group of rats (FIG. 4).

TABLE 3
Plasma and tumor exposure levels in naïve, UM-UC-1 orthotopic, or UM-UC-1 s.c.
tumor-bearing nude rats following a single oral or intravesical dose of erdafitinib.

| | | | | | | |
|---|---|---|---|---|---|---|
| Tumor | Dose | | | Plasma concentration | Tumor concentration | Tumor: |
| site | (mg/kg) | Route | N | (ng/g) | (ng/g) | plasma ratio^{c} |

Time post dose (hours)

| | | | | 2 | 7 | 2 | 7 | 2 | 7 |
|---|---|---|---|---|---|---|---|---|---|
| s.c. | 20 | p.o. | 3 | 468.7 | 86.4 | 1,242.3 | 453.7 | 2.7 | 5.2 |
| | | | | (187.7) | (64.8) | (321.7) | (60.1) | | |
| Bladder | 20 | p.o. | 3 | 2,850.7 | 1,454.8 | 3,321 | 983.3 | 1.2 | 0.7 |
| | | | | (2,189.1) | (2,352.1) | (1,625.3) | (661.8) | | |
| Bladder | 6 | IVES | 3 | 26.6 | 2.1 | 3,148.7 | 2,050.7 | 118.4 | 957.2 |
| | | | | (19.1) | (2.0) | (3,314.3) | (2,802.1) | | |
| Naïve | 6 | IVES | 3 | 1.9 | 0.6 | — | — | — | — |
| | | | | (1.8) | (0.3) | | | | |

HP-β-CD, hydroxypropyl β-cyclodextrin; IVES, intravesical (bladder instillation); LLOQ, lower limit of quantitation; PO or p.o., oral.
Values are mean with standard deviation in parentheses. Mean exposures for 48- and 120-hour timepoints were low (<35 ng/g). Erdafitinib was dosed as a 10% w/v HP-β-CD solution.
^{a}LLOQ serum = 0.05 ng/mL
^{b}LLOQ tumor = 2 ng/g
^{c}Average = tumor concentration (ng/g)/plasma concentration (ng/ml)

The effects of a single 20 mg/kg oral or 6 mg/kg intravesical dose of erdafitinib on ERK1/2 phosphorylation in orthotopic bladder UM-UC-1 tumors was assessed at various timepoints by capillary immunoblotting. Proteins from tumor sample lysates collected 2, 7, 48, and 120 hours after treatment with erdafitinib or vehicle were separated by capillary electrophoresis and probed with antibodies detecting phosphorylated (p)ERKI/2 and total ERK1/2. The signals for pERK1/2 were divided by the total ERK1/2 signal in the same sample and the mean of the pERK1/2 values for the corresponding vehicle-treated samples was set at a relative value of 1. At each timepoint, the pERK/ERK ratio for each tumor sample was divided by the mean pERK/ERK ratio derived from the corresponding control samples. There was 1 exception, as there were no vehicle-treated samples at the 120-hour timepoint, the pERK/ERK ratios for the erdafitinib-treated samples at the 120-hour timepoint were divided by the mean of the 48-hour vehicle-treated group.

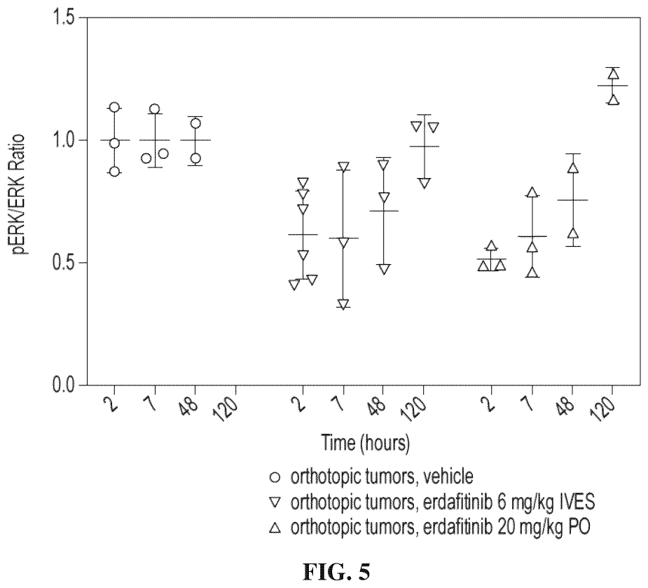

The 6 mg/kg intravesical and 20 mg/kg oral doses of erdafitinib both resulted in a statistically significant decrease in ERK1/2 phosphorylation in UM-UC-1 tumors at 2 hours post dosing (FIG. 5, Table 4). Although not statistically significant, pERK levels were also lower in the erdafitinib-treated tumors compared to vehicle treated tumors at 7 and 48 hours, whereas by 120 hours the levels of pERK 1/2 were comparable to those of vehicle-treated rats.

TABLE 4
Levels of pERK/ERK in UMUC1 orthotopic tumors following a single
oral or intravesical dose of erdafitinib.

| Time | | | | | | |
|---|---|---|---|---|---|---|
| post dose | Route of | Dose | | | | |
| (hours) | administration | (mg/kg) | N | pERK/ERK^{a} | | p-value^{b} |
| 2 | IVES | 0 | 3 | 1 | (0.13) | NA |
| 2 | IVES | 6 | 6 | 0.62 | (0.18) | 0.0102 |
| 2 | p.o. | 20 | 3 | 0.52 | (0.05) | 0.0063 |
| 7 | IVES | 0 | 3 | 1 | (0.11) | NA |
| 7 | IVES | 6 | 3 | 0.60 | (0.28) | 0.0841 |
| 7 | p.o. | 20 | 3 | 0.61 | (0.17) | 0.0893 |
| 48 | IVES | 0 | 2 | 1 | (0.10) | NA |
| 48 | IVES | 6 | 3 | 0.71 | (0.22) | 0.2682 |
| 48 | p.o. | 20 | 2 | 0.76 | (0.19) | 0.4065 |
| 120 | IVES | 6 | 3 | 0.98 | (0.13)^{c} | 0.9630 |
| 120 | p.o. | 20 | 2 | 1.22 | (0.07)^{c} | 0.1823 |

ANOVA, analysis of variance; ERK, extracellular signal-regulated kinase; IVES, intravesical; NA, not applicable; pERK, phosphorylated extracellular signal-regulated kinase; p.o., oral.
The “zero dose” animals were dosed with vehicle.
^{a}Ratio of pERK/ERK mean group values divided by the mean of the vehicle-treated group.
Standard deviation between brackets.
^{b}One-way ANOVA, Dunnett's test for multiple comparison.
^{c}Values divided by mean of the 48-hour vehicle-treated group.

Overall, these data demonstrate that intravesical administration of erdafitinib provides adequate tumor PK/PD while dramatically reducing exposures in plasma, thereby decreasing potential for on-target, off-tumor toxicities compared to oral therapy.

### Example 5: Continuous Perfusion Studies in Orthotopic Bladder Cancer Model

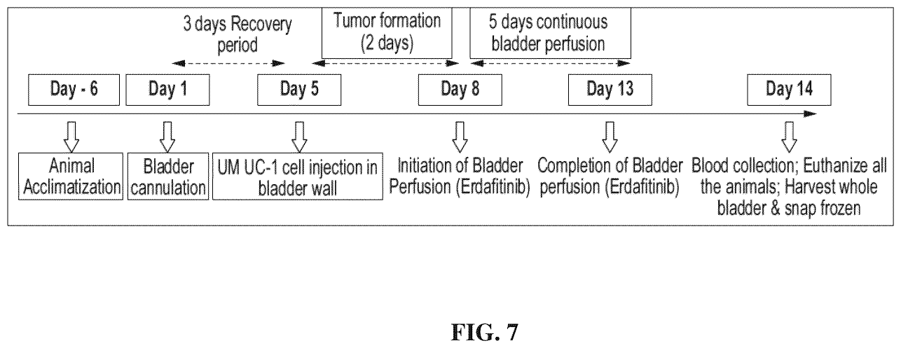

Perfusion studies: The bladders of the study animals were cannulated on Day 1 and the animals were allowed to recover for 3 days. On Day 5, UM-UC-1 cells (2×10⁶ cells) were injected into the lateral wall of each bladder. Following a 2-day tumor growth period, erdafitinib was perfused continuously for 5 days, followed by necropsy within 24 hours. Based on the in vitro results, target urine concentrations of 0.5, 1.0, and 5.0 μg/mL were used in the perfusion experiments. The study design is shown in FIG. 7. Body weight, daily urine production, and daily water consumption were recorded. At necropsy, a plasma sample, bladder photographs, and bladder weight measurements were recorded. Post necropsy total bladder weight, which is comprised of normal bladder tissue plus urothelial tumor, was used to determine the effect of erdafitinib on tumor growth.

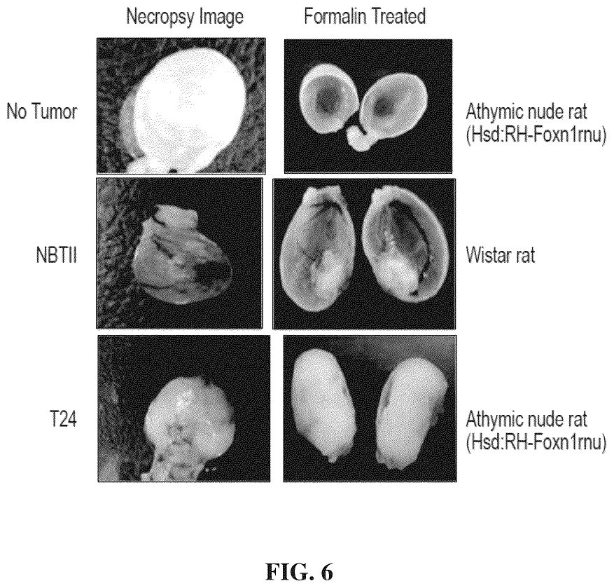

Human-derived tumor cells, when implanted into the bladder wall of athymic rats, grew rapidly. Within 7 days of implantation, the tumor occupied the majority of the urothelial surface and increased total bladder weight up to 7-fold (FIG. 6). Thus, total bladder weight is an accurate measure of drug response.

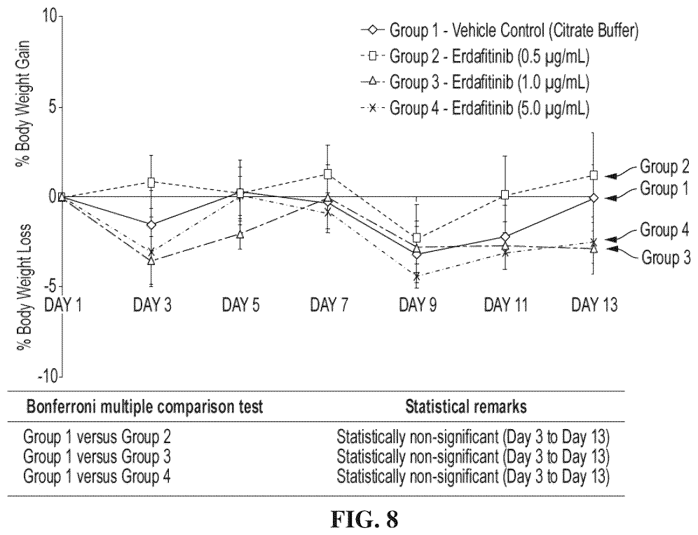

Five days of continuous erdafitinib perfusion at nominal urine concentrations of 0.5, 1.0, and 5.0 μg/mL was generally well tolerated. Body weight changes during the study are shown in FIG. 8. An initial small reduction in body weight, <5% (Day 1-3), was seen in most groups including vehicle control due to the impact of the bladder cannulation surgery. Minimal body weight changes were noted after intra-bladder tumor cell injection on Day 5. Transfer to metabolism cages and initiation of the bladder perfusions resulted in a second small body weight decrease unrelated to perfusate drug concentration. Based on cage side observations, no visible signs of abnormal behavior or clinical symptoms were observed in any of the treatment groups.

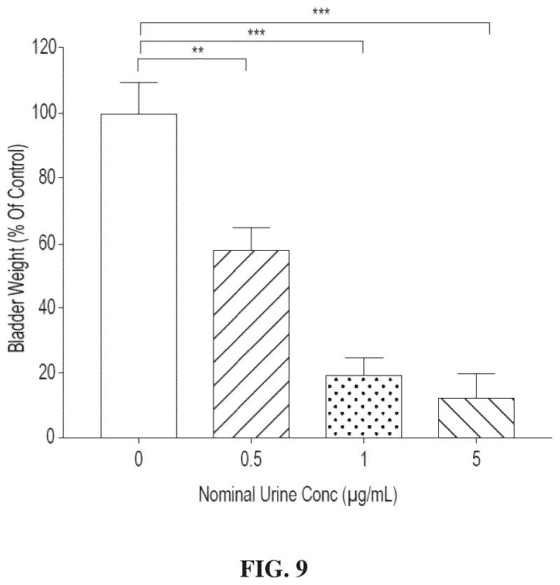

The percent reduction in relative tumor weight between the control group and the drug perfusion groups was determined as an initial measure of efficacy. Bladder tissue and tumor samples were also submitted for analysis of FGFR signaling activity by determining the phosphorylated fibroblast growth factor receptor substrate (FRS)2a levels and the pERK to ERK ratio. Additional urine, plasma, and bladder samples were collected to determine erdafitinib concentration using established liquid chromatography—tandem mass spectrometry (LC-MS/MS) methods. The mean bladder weight change in animals receiving different erdafitinib concentrations is shown in FIG. 9. A significant dose-related decrease in bladder weight was observed in the erdafitinib treatment groups (**p<0.01 for erdafitinib at 0.5 μg/mL, ***p<0.001 for erdafitinib at 1.0 and at 5 μg/mL nominal urine concentrations) when compared to the vehicle control group

### Example 6: Dose-Response Evaluation of Erdafitinib in Bladder-Perfused Athymic Rats with RT-112 Implanted into the Bladder Wall

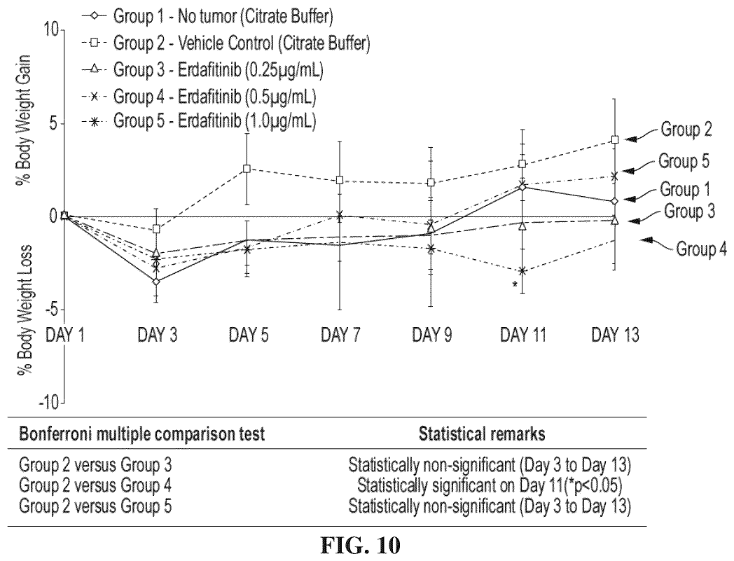

Perfusion studies: The experimental study design was the same as described in Example 5 (FIG. 7), except that the perfusions were continued until the time of necropsy on Day 14. Six days of continuous perfusion treatment with erdafitinib at the nominal urine concentrations of 0.25, 0.5, and 1.0 μg/mL was well tolerated during the experimental period Body weight changes observed during the study are shown in FIG. 10. Mild body weight loss was observed during study conduct, with maximum mean values ranging up to −3% in the erdafitinib-treated group on Day 11.

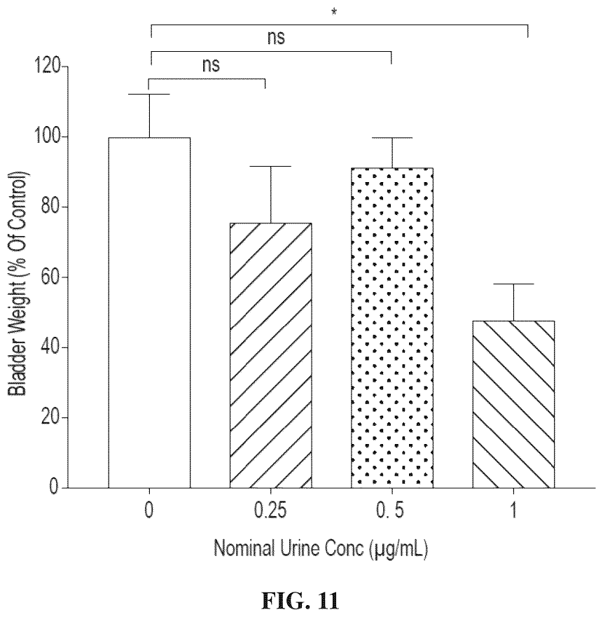

The effect of intravesical erdafitinib exposure on tumor growth as determined by changes in total bladder weight is shown in FIG. 11. Mean bladder weights trended lower with increasing erdafitinib concentration, but decreases were not statistically significant for the 0.25 and 0.5 μg/mL dose groups relative to vehicle control animals. A significant bladder weight reduction (*p<0.05) was observed in animals receiving the 1.0 μg/mL perfusate concentration compared to vehicle control.

Dose-response evaluation of erdafitinib (0.25-5 μg/mL) in bladder-perfused athymic rats with UM-UC-1 or RT-112 cell lines implanted into the bladder wall demonstrated that the dosing regimen of erdafitinib was generally well tolerated. Significant dose-dependent bladder weight decreases were observed in the erdafitinib treatment groups when compared to the vehicle control group, demonstrating the bladder perfusion of erdafitinib reduced tumor growth.

### Example 7: Intravesical Pharmacokinetic and Distribution Studies in Rats and Minipigs

Systemic and urinary bladder PK studies were conducted following single intravesical (bolus) administration of erdafitinib in solution (HP-β-CD) formulation to rats and minipigs. These studies were initially conducted to determine drug exposures in bladder tissue, urine, and plasma. Additionally, bladder tissue was evaluated for gross and microscopy examination to determine whether there were any local effects of the drug or formulation in the study. The goal of Example 7 was to determine the feasibility of erdafitinib intravesical therapy with bladder installation of erdafitinib.

Single intravesical dose PK in rats: Systemic and urinary bladder PK of erdafitinib was determined in female Sprague-Dawley rats after intravesical administration of erdafitinib solution at 2, 6, and 18 mg per kg body weight. Rats were kept under anesthesia using isoflurane (2-4%), a catheter was introduced into the urethra to the bladder, and erdafitinib solution (10% w/v HP-β-CD in citrate buffer pH 5.5) was instilled to the rat bladder via this catheter. Solution formulations were prepared at various strengths in order to administer 0.5 mL volume to each rat bladder to dose 2, 6, and 18 mg/kg. Corresponding doses in nominal amount of drug were 0.5, 1.5, and 4.5 mg, respectively After 1 hour of post-installation, rats were transferred to metabolic cages for collection of samples for PK determination. Blood samples were taken from the tail vein at 24, 48, 72, 96, and 168 hours after completing the 1-hour contact time of compound after dosing (3 rats per time point). At each blood sampling time point, bladder samples were taken from each rat for drug analysis. In addition, bladders collected at 96 hours in the 18 mg/kg dose (high-dose) group were microscopically evaluated. Urine collections were limited to initial 0-6 hours post dose from all rats.

In plasma, almost all samples were below the quantification limit (0.02 ng/mL) for the 2 and 6 mg/kg dose group. For the 18 mg/kg dose group, some measurable concentrations were observed at 24, 48, and 72 hours (between 0.0303 and 0.106 ng/mL), but at 96 and 168 hours all samples were below the quantification limit. In the bladder, concentrations could be measured up to 72 hours for the 2 and 6 mg/kg dose group, and up to 168 hours for the 18 mg/kg dose group. Concentrations were highest at the 24-hour time point and declined thereafter. No dose-linearity could be observed. Exposures were similar between the tested doses. The percentage of compound eliminated as unchanged drug in the urine (in the first 6 hours), amounted to 23.5%, 19%, and 50.3% for the 2, 6, and 18 mg/kg dose group.

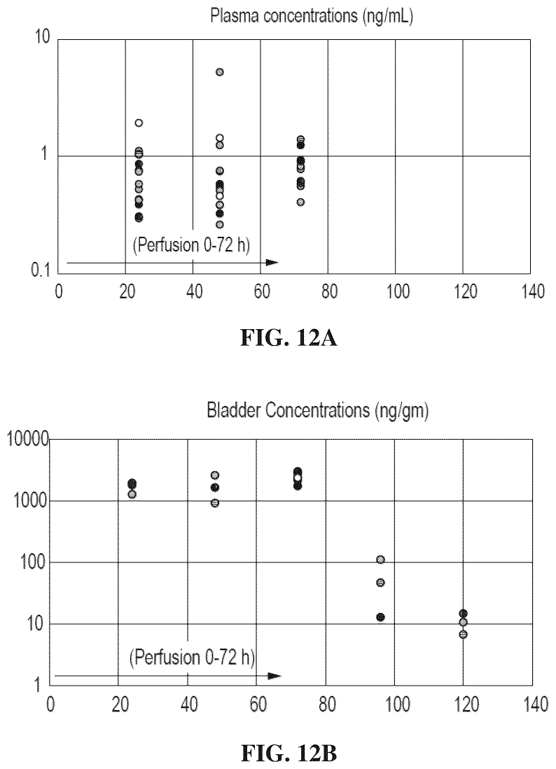

Systemic and bladder PK of continuous intravesical erdafitinib in rats: Systemic and bladder PK of erdafitinib was determined in female Sprague-Dawley rats after continuous intravesical infusion of an aqueous solution of erdafitinib. Rat bladders (5 groups of rats, n=3/group) were surgically catheterized under anesthesia and the catheter was externalized, tunneled subcutaneously, and connected to a vascular access harness (VAH) in the neck. Rats were transferred to individual metabolic cages with free access to food and water during the 1-week post-surgery recovery period. On the study day erdafitinib solution (0.1 mg/mL, 0.1 mL/hour, citrate buffer pH 5.5 containing 5% w/v HP-β-CD) was perfused through rat bladders via catheter for over 72 hours. The first group (n=3) was sacrificed at 24 hours post perfusion and 2 of the 4 remaining groups were sacrificed at 48 and 72 hours, post perfusion. Perfusion was stopped at 72 hours for the last 2 groups, and these groups were sacrificed at 96 and 120 hours to determine the drug elimination phase from bladder. Plasma and bladder samples were collected at all time points. Urine was collected from the third group during the 48-72 hours perfusion period for drug analysis. Plasma concentrations in rats following 72-hour bladder perfusion of erdafitinib solution (0.1 mg/mL, 0.1 mL/hour, cumulative dose 0.72 mg) are shown in FIG. 12A There were no levels detected (below quantification limit; 0.2 ng/mL) in samples up to 120 hours (i.e., additional 48 hours) after stopping the perfusion at 72 hours. Bladder levels in rats following 72-hour bladder perfusion of erdafitinib solution (0.1 mg/mL, 0.1 mL/hour, cumulative dose 0.72 mg) are shown in FIG. 12B. Rat bladders perfused with 0.1 mg/mL erdafitinib solution did not show changes, and this formulation strength was considered tolerable. Average daily urine concentration was measured at about 10,000 ng/mL for urine collected during the perfusion interval of 48-72 hours.

TABLE 5
Plasma and bladder systemic exposure following bladder perfusion
of erdafitinib.

| Parameter | Plasma | Bladder | Concentration | Plasma | Bladder |
|---|---|---|---|---|---|
| AUC₀₋₂₄ₕ | 8.09 | 20,200 | C₂₄ₕ (ng/mL) | 0.674 | 1,680 |
| (ng · h/mL) | | | | | |
| AUC₀₋₄₈ₕ | 27.2 | 61,100 | C₄₈ₕ (ng/mL) | 0.916 | 1,730 |
| (ng · h/mL) | | | | | |
| AUC₀₋₇₂ₕ | 48.1 | 111,000 | C₇₂ₕ (ng/mL) | 0.829 | 2,420^{a} |
| (ng h/mL) | | | | | |
| AUC₀₋₉₆ₕ | 48.1 | 126,000 | C₉₆ₕ (ng/mL) | BQL | 56.9 |
| (ng · h/mL) | | | | | |
| AUC₀₋₁₂₀ₕ | 48.1 | 127,000 | C₁₂₀ₕ (ng/mL) | BQL | 10.7 |
| (ng · h/mL) | | | | | |

AUC₀₋ₓₕ, area under the plasma concentration − time curve from time of administration until x hours; Cₓₕ, plasma concentration at time x; BQL, below quantification limit.
^{a}Average daily urine concentrations at this time point was about 10,000 ng/mL.

Results indicated marked bladder tissue uptake of erdafitinib and maintenance of high bladder levels with minimal systemic exposure upon continuous slow-rate perfusion of erdafitinib.

Systemic and bladder PK of continuous intravesical erdantinib in pig: Systemic and bladder PK of erdafitinib was assessed in five female pigs (Domestic Yorkshire Crossbred swine) after continuous intravesical infusion of an aqueous solution of erdafitinib. On Day −7 a catheter was surgically placed in the bladder of each animal. The distal end of the catheter was anchored to a subcutaneous site and attached to a vascular access port (VAP). The port was secured, and the animals were allowed to recover. Each animal was subsequently fitted with a portable infusion pump that was attached to the bladder catheter via the VAP. Dose formulations (22.5 μg/mL erdafitinib solution in 50 mM citrate buffer pH 6.0) were prepared every day and sterile filtered daily and analyzed to confirm the concentration. Dose formulation was perfused into the bladder at a constant rate of 12.5 mL/hour for 6 consecutive days for 2 animals and 8 consecutive days for 3 animals. All excreted urine was collected in 24-hour intervals through Day 6 or 8. Blood samples were collected daily on study Days 1 to 8. Bladder tissue samples were collected from each animal at necropsy. Samples obtained from all animals were analyzed for erdafitinib using qualified LC-MS/MS methods. Based on the formulation analysis on all days, the average daily administered dose for each animal ranged from 7.06 to 7.56 mg and overall mean dose was 7.3 mg/day. Based on the average bodyweight (pre dose), administered dose was 0.22 mg/kg/day.

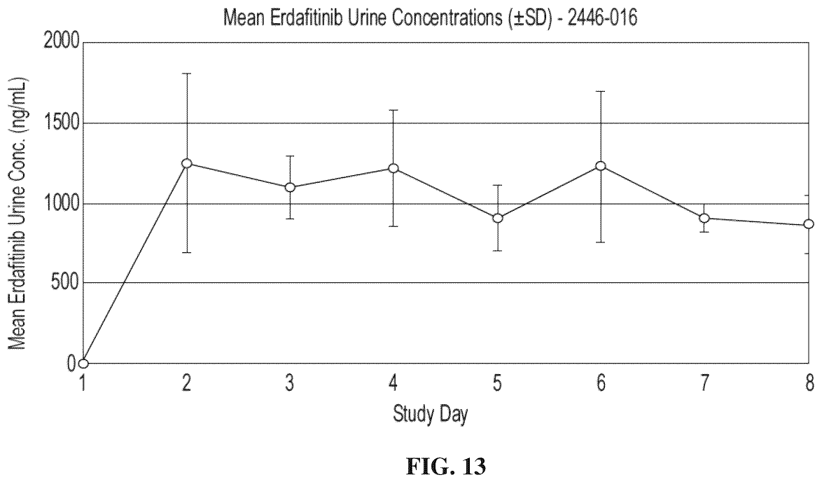

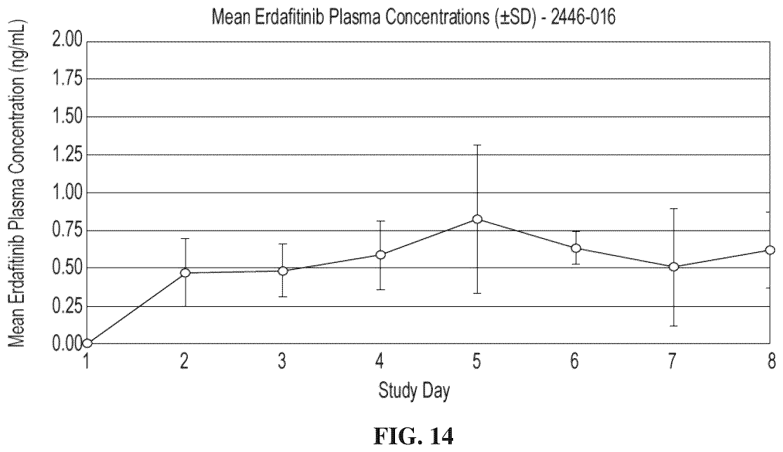

Mean (±SD) erdafitinib urine concentrations ranged from 1,255±554 to 873±179 ng/mL on Days 2 to 8 (FIG. 13). Mean (±SD) erdafitinib plasma concentrations on Days 2 to 8 are presented in FIG. 14 and averaged (±SD) 0.622±0.250 to 0.828±0.487 ng/mL. Over the 7-day period of this study, the average (±SD) daily urine output was 966±253 mL. Although inter- and intra-animal variation in daily urine production was observed, no significant trends in urine production were observed over the treatment period. Erdafitinib urinary recoveries were relatively consistent in all animals, averaging 910±812 to 1,135±760 ng on Days 2 to 8. Daily erdafitinib recoveries averaged 15.7%±5.67% of the average daily amount of erdafitinib administered.

Mean erdafitinib concentrations in full thickness bladder tissues were measured on Day 6 and Day 8 (end of perfusion) and the values ranged from 315 to 998 ng/g and 346 to 2,688 ng/g, respectively. Erdafitinib concentration was measured in urothelium and underlying tissue layers (i.e., muscle). These data suggest a >10-fold concentration of erdafitinib in the urothelial layer of the bladder when compared to the underlying tissue layers, suggestive that the drug was mainly retained in the urothelium.

TABLE 6
Erdafitinib bladder tissue concentrations in pig following bladder perfusion of
erdafitinib for 7 days.

| | | | | | | | Mean bladder |
|---|---|---|---|---|---|---|---|
| | | | | | | | conc/mean |
| | | | | | | Mean | urine conc |
| | Erdafitinib concentration (ng/g) | | | | Mean | bladder | ratio by |
| | Bladder tissue | | | | urine | conc/mean | perfusion |
| Bladder | | Overall | Urothel | Rem | conc | urine conc | duration |
| tissue | Mean | mean | layer | layer | (ng/m L) | ratio | Day 8 |
| Animal 1001, Day 8 | | | | | | | |
| Dome | 2,620 | 1,913 | 4,520 | 147 | 991.9 | 1.93 | 2.33 |
| Lateral wall | 1,850 | | 3,290 | 198 | | | |
| Trigone | 1,270 | | 2,330 | 66.4 | | | |
| Animal 1002, Day 8 | | | | | | | |
| Dome | 2,527 | 2,688 | 5,170 | 737 | 578.9 | 4.64 | |
| Lateral wall | 4,127 | | 11,600 | 382 | | | |
| Trigone | 1,411 | | 174 | 1,740 | | | |
| Animal 1003, Day 8 | | | | | | | |
| Dome | 469 | 346 | 277 | 25.1 | 849.1 | 0.41 | |
| Lateral wall | 422 | | 612 | 37.4 | | | |
| Trigone | 145 | | 227 | 19.7 | | | |
| Animal 1004, Day 8 | | | | | | | |
| Dome | 1,240 | 998 | 6,900 | 299 | 1,044 | 0.96 | 0.60 |
| Lateral wall | 1,193 | | 7,490 | 298 | | | |
| Trigone | 561 | | 1,390 | 118 | | | |
| Animal 1005, Day 8 | | | | | | | |
| Dome | 334 | 315 | 1,420 | 125 | 1,268 | 0.25 | 1.30 |
| Lateral wall | 372 | | 2,170 | 111 | | | |
| Trigone | 238 | | 439 | 38.6 | | | |

conc, concentration; Rem layer, bladder underlying tissue layer; Urothel layer, bladder urothelium.

### Example 8: Stability and Protein Binding Studies

Stability in urine: Urine was spiked with erdafitinib at 1, 3, and 5 μg/mL and incubated (in triplicates) at 37° C. for 6 hours as part of the equilibrium dialysis study. At the end of 6-hours post-dialysis incubation, drug was analyzed, and the recovery was calculated against the spiked concentrations. The percent recovery of erdafitinib in the study was ranging from 89% to 98% in human urine, 90% to 95% in rat urine, and 92% to 93% in minipig urine, indicating that erdafitinib is stable in urine. These results suggest that erdafitinib will remain stable in urine in the bladder to provide exposure to tumor and bladder tissue.

### Example 9: Prototype Development

Based upon experiments, including animal studies, release rates of 1 mg/day, 2 mg/day 4 mg/day, and 6 mg/day were selected for further development. Designs enabling 30-day and 90-day use durations were evaluated. The 30-day designs were engineered to provide higher drug release rates that also exceeded the device 90-day payload capacity. The minimum target release rate was defined as the rate required to yield average erdafitinib urine concentrations of 1 g/mL. Higher release rates were also evaluated to increase tumor exposures and to assess local tolerability and systemic exposure liabilities. Additional performance metrics included urine pH, urine volume, and urine composition independence.

Erdafitinib exhibits significant pH-dependent solubility over the normal urine pH range of 5.5 to 7. As a result, different drug formats and minitablet excipient combinations were evaluated to minimize the effect of urine pH and composition on system release rate.

A factorial design-based screen was first completed to evaluate the complete range of possible release rates and pH effects. Approximately 900 combinations of device polymers and erdafitinib drug formats were tested using powder packed, short core systems, which were 2-cm versions known to accurately scale to the full-length 15-cm design. The drug formats evaluated included erdafitinib free base, erdafitinib HCl salt, erdafitinib L-lactate salt, and erdafitinib free base plus HP-β-CD.

### Material Screening

In general, materials that are impermeable to erdafitinib, the API, are suitable for use in the osmotic system. Platinum cured silicone, thermoplastic polyurethanes (TPUs), and ethylene vinyl acetate (EVA) materials were screened (Table 7). The test articles were filled with formulated API (powder or tablets), sealed, placed in foil pouches, and gamma irradiated (nominal 35 kGy). The systems were placed into simulated urine (pH 6.8), stored at 37° C., and sampled periodically. The amount of API in each sample was determined by high-performance liquid chromatography (HPLC) analysis.

TABLE 7
Materials screened.

| | | Test | | |
|---|---|---|---|---|
| | Raw material | article | | |
| Material | manufacturer | form | Hardness | Description |
| Silicone | NuSil | Tube | 50A | High-consistency silicone |
| | | | | elastomer, platinum cured, |
| | | | | MED-4750 |
| TPU | Lubrizol | Tube | 70A | Carbothane ™ Aliphatic, |
| | | | | PC-3575A |
| | | | 62A | Tecothane ™ Soft, AR-62A |
| | | | 42D | Tecophilic ™, HP-60D-35 |
| | | | 77A | Carbothane ™ Aromatic, |
| | | | | AC-4075A |
| | | | | Tecothane ™, TT-1074A |
| | | | 75A | Tecophilic ™, HP-93A-100 |
| | | | 83A | Tecoflex ™, EG-80A |
| | | | 72A | |
| EVA | 3M | Film | NA | CoTran ™, 9712, 0.05 mm |
| | | | | thick film |

EVA, ethylene vinyl acetate;
NA, not available;
TPU, thermoplastic polyurethane

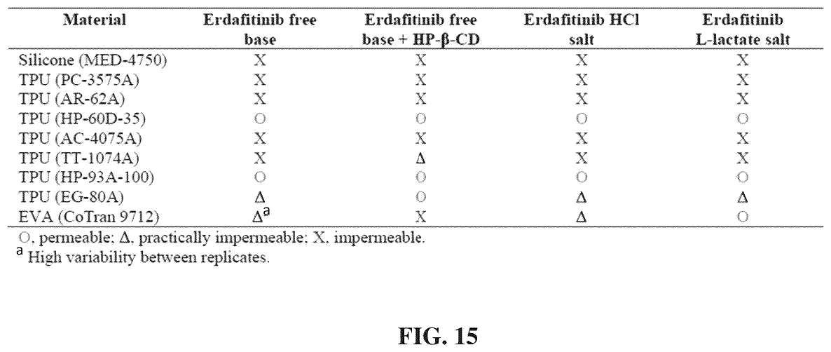

Permeability screening results are shown in FIG. 15. Materials that were suitable for use in an osmotic system were impermeable to the API. Silicone is used as the tubing material for the osmotic system and is also suitable as an osmotic tubing material for all 4 erdafitinib forms. Based on its impermeable properties, silicone was selected as the material for further development of an osmotic prototype.

### Osmotic Systems

Silicone tubing was selected for osmotic systems, and 4 API forms were screened with and without additional osmotic agents capable of producing constant water flux through silicone tubing for at least 30 days (e.g., sodium chloride, potassium chloride, potassium bicarbonate, sodium phosphate dibasic dihydrate, sodium sulfate, and sodium L-(+)-tartrate dihydrate). Short core osmotic systems were tested with the 4 API forms alone and with added osmotic agents. The HCl salt form was not tested with sodium chloride or potassium chloride due to the common ion between the API and the osmotic agent. The short core system that showed most promising release rates was the L-lactate salt API form, with no added osmotic agent.

TABLE 8
Summary of prototypes tested in minipigs.

| Prototype | Group | Prototype description |
|---|---|---|
| 4 | 4 | Osmotic (orifice + end plugs), 0.2 mm wall, |
| | | erdafitinib L-lactate, tablets |
| | | (20 wt % water-insoluble excipients) |
| 5 | 5 | Osmotic (orifice + end plugs), 0.2 mm wall, |
| | | erdafitinib L-lactate, tablets |
| | | (water soluble excipients) |

Osmotic Systems with Erdafitinib L-Lactate Salt

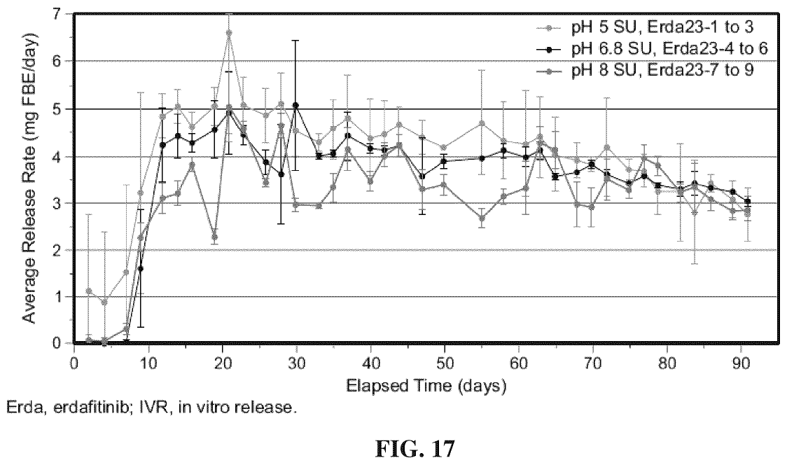

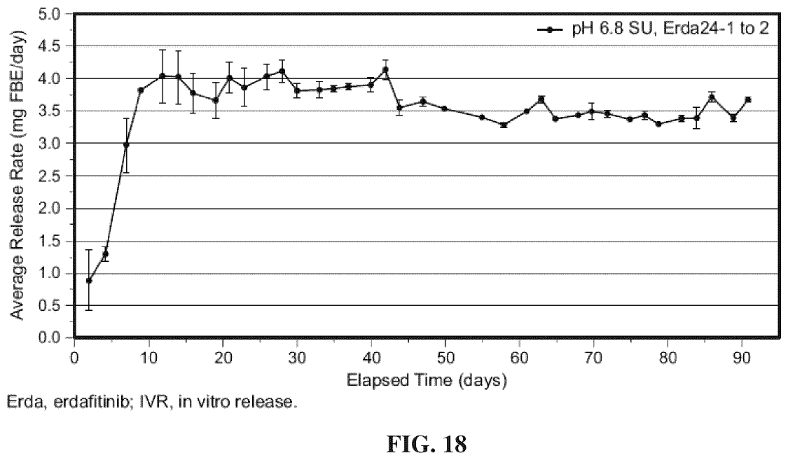

Two osmotic system prototypes with erdafitinib L-lactate salt were tested in minipigs (Prototype 4). Osmotic (orifice+end plugs), 0.2 mm wall, erdafitinib L-lactate, tablets (20% insoluble); and Prototype 5: Osmotic (orifice+end plugs), 0.2 mm wall, erdafitinib L-lactate, tablets (soluble)); see FIGS. 16A and 16B). The drug constituent formulations are described in Table 9. Both prototypes contained a dual lumen silicone tube (2.64 mm large lumen ID, 0.2 mm wall thickness), a 150 μm orifice, 2 end plugs, a 15 cm drug core, and a wireform as the retentive feature. Table 10 describes the drug constituent payload for each prototype. IVR testing was performed with Prototype 4 using simulated urine at 3 different pH ranges as the release media: pH 5, 6.8, and 8 (n=3 systems tested in each media). Prototype 5 was tested in simulated urine at pH 6.8 (n=2 systems tested in IVR). The IVR profile for Prototype 4 showed good agreement between pH 5, 6.8, and 8 simulated urine, and nearly zero-order release over the 90-day duration (FIG. 17). The IVR profile for Prototype 5 was tested in pH 6.8 simulated urine, with only 2 replicates (FIG. 18). This prototype also showed approximately zero-order release over the 90-day duration.

TABLE 9
Erdafitinib mono L-lactate minitablet compositions (drug constituents).

| | | | |
|---|---|---|---|
| | (Prototype 4) | (Prototype 5) | |
| Component | % w/w | 0% w/w | Function |

Intragranular

| | | | |
|---|---|---|---|
| JNJ42756493-AFK | 80 | 90.8 | API |
| (erdafitinib mono L-lactate) | | | |
| Microcrystalline cellulose | 18 | NA | Binder |
| (Avicel PH 105) | | | |
| Polyvinylpyrrolidone | NA | 4.7 | Binder |
| Colloidal silicon dioxide | 0.3 | NA | Glidant |
| (Aerosil 200) | | | |
| Magnesium stearate | 0.75 | NA | Lubricant |
| (Ligamed MF-2V) | | | |

Extragranular

| | | | |
|---|---|---|---|
| Colloidal silicon dioxide | 0.2 | NA | Glidant |
| (Aerosil 200) | | | |
| Magnesium stearate | 0.75 | NA | Lubricant |
| (Ligamed MF-2V) | | | |
| Polyethylene glycol (PEG8k) | NA | 4.5 | Lubricant |
| Total | 100 | 100 | |

API, active pharmaceutical ingredient; NA, not applicable; w/w, weight per weight.

TABLE 10
Final system parameters.

| Parameter | Prototype 4 | Prototype 5 |
|---|---|---|
| Tablet mass | 932 mg | 961 mg |
| API mass | 620 mg FBE | 726 mg FBE |

FBE, free base equivalents.

### Pharmacokinetic Evaluation in Minipigs

Osmotic design released erdafitinib at rates designed to provide the target urine concentration of 1 μg/mL or greater in minipigs. The zero-order systems demonstrated in vitro release rates (defined as release at a constant rate for at least 30 days) of up to 2 mg/day (>90-day duration). Inter- and intra-device release rate variance was lowest with the zero-order systems.

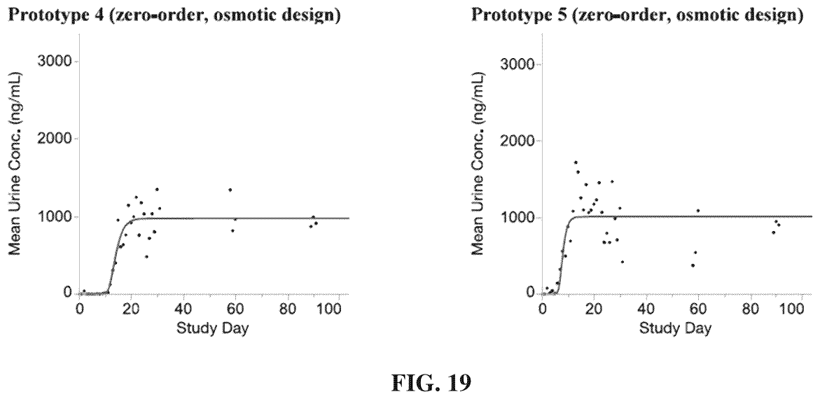

Based on the short core data, a series of 200 full-length systems were developed and tested confirming the short core results. A subset of these were tested in minipigs to determine the in vivo release rate characteristics of the osmotic design and to obtain the release rates required to achieve target urine concentrations. The in vivo results largely confirmed the in vitro findings and demonstrated 2 to 4 mg/day release rate in vitro was sufficient to maintain target erdafitinib urine concentrations. FIGS. 17 and 18 summarize the in vitro release characteristics of representative osmotic systems selected for minipig testing. FIG. 19 summarizes the urine concentration versus time profiles of the same systems. Prototypes 4, and 5 were representative zero-order osmotic designs, utilizing osmotic-based release.

### Example 10: Chemical Properties, Stability, Formulation, and Device Development

Erdafitinib was assigned the number JNJ-42756493 with suffix -AAA denoting the free base form and suffix -AFK denoting the mono L-lactate salt.

A summary of solid-state properties of both the free base and L-lactate salt is provided in Table 11. The physical stability of the L-lactate salt was evaluated by differential scanning calorimetry (DSC), X-ray powder diffraction (XRPD), thermogravimetric analysis (TGA), and infrared (IR) after storage in open dish at different conditions for 6 weeks. The product was found to be crystallographically stable. No evidence of dissociation or form conversion was found (Table 12).

TABLE 11
Overview solid state properties free base and L-lactate salt
of JNJ-42756493.

| Parameter | Free base (AAA) | mono L-lactate salt (AFK) |
|---|---|---|
| Melting point | 141° C. | 173.4° C. |
| TGA | No weight loss between | No weight loss between RT |
| | RT and 141° C., the | and 140° C., then a 16% |
| | melting temperature | weight loss indicative of |
| | | dissociation/decomposition |
| DVS | Non-hygroscopic | Non-hygroscopic |
| XRPD | Crystalline | Crystalline |

DVS, dynamic vapor sorption; RT, room temperature; TGA, thermogravimetric analysis; XRPD, X-ray powder diffraction.

TABLE 12
Solid state stability summary of mono L-lactate salt
JNJ-42756493-AFK.

| | TGA weight loss | | | DSC Max |
|---|---|---|---|---|
| Condition | % w/w <135° C. | XRPD | IR | (° C.) |
| 0 days | 0.2 | Cryst., Ref | Cryst., Ref | 173.4 |
| RT/<5% RH | 0.0 | ~Ref | ~Ref | 173.8 |
| RT/56% RH | 0.1 | ~Ref | ~Ref | 174.2 |
| RT/75% RH | 0.0 | ~Ref | ~Ref | 173.9 |
| 50° C. | 0.0 | ~Ref | ~Ref | 174.2 |
| 40° C./75% RH | 0.0 | ~Ref | ~Ref | 174.3 |

Cryst.Ref., crystalline reference; DSC, differential scanning calorimetry; IR, infrared; Max, maximum; RH, relative humidity; RT, room temperature; TGA, thermogravimetric analysis; w/w, weight per weight; XRPD, X-ray powder diffraction.

### Formulation Device Development

Formulation development was focused on erdafitinib L-lactate minitablet concepts reference is made to Table 13 for an exemplary concept).

TABLE 13
JNJ-42756493-AFK (erdafitinib mono L-lactate)
minitablet composition.

| Component | % w/w | Function |
|---|---|---|
| API granulate | 71.43 (70.00, 1.43) | API, |
| (erdafitinib L-lactate, HPMC E15) | | Binder |
| Kollidon VA64 | 12.91 | Binder |
| MCC - Avicel PH105 | 12.91 | Binder |
| Colloidal silicon dioxide (Aerosil 200) | 0.25 | Glidant |
| Magnesium stearate | 2.5 | Lubricant |
| Total | 100 | |

API, active pharmaceutical ingredient, w/w, weight per weight.

The osmotic silicone system (osmotic pressure driven, with orifice) was evaluated.

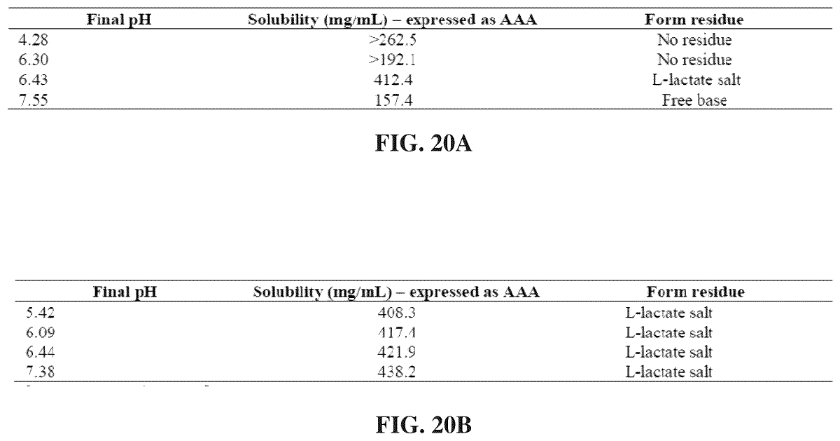

The L-lactate salt has an unexpected very high solubility over a broad pH range and unexpectedly has osmotic properties. Solubility of the L-lactate salt as function of pH in water (FIG. 20A) and simulated urine (FIG. 20B) at 37° C. is shown.

### Example 11: Synthesis of Erdafitinib L-Lactate

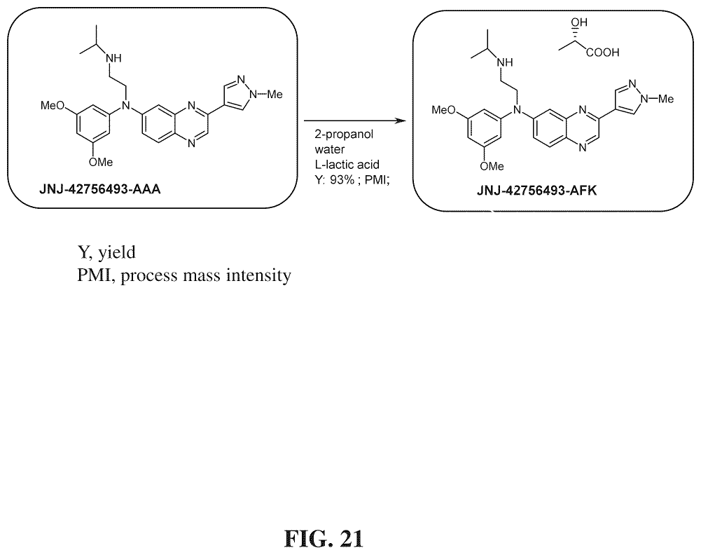

Synthesis of Erdafitinib L-lactate salt: Erdafitinib mono L-lactate salt was successfully produced via salt formation of erdafitinib free base with solid mono L-lactic acid (FIG. 21). Crystallization was performed with seeding with API.

### Procedure 1

TABLE 14
Material list:

| Material | MW | Ratio | |
|---|---|---|---|
| JNJ-42756493-AAA | 446.55 | 1 | mol |
| L-lactic acid | 90.08 | 1.02 | mol/mol |
| Isopropanol (IPA) | 60.1 | 1.006 | L/mol |
| Water | 18.02 | 0.11178 | L/mol |
| Isopropanol (IPA) | 60.1 | 1.1178 | L/mol |
| JNJ-42756493-AFK | 536.63 | 5.4 | g/mol |
| Isopropanol (IPA) | 60.1 | 0.54 | L/mol |

Procedure with Wet Milling:

### Dissolve API

Added erdafitinib base to reactor.

Added 1.02 mol L-lactic acid/mol erdafitinib base.

Added 1.006 L Isopropanol/mol erdafitinib base to the reactor

Added 0.11178 L water/mol erdafitinib base to the reactor.

Dissolved at 70° C. and hold. Visually confirmed complete dissolution.

Set receiver vessel to jacket temperature 80° C.

Performed polish filtration and kept polish filter at least at 70° C.

Rinsed filter with 1.1178 L Isopropanol/mol erdafitinib base.

Waited for until temperature in receiver reactor stabilized to 80° C.

### Establish Seeding Conditions

Cooled to 69° C. at 0.3° C./min

Waited until temperature stabilized

Seeded with 1 m % seed material (5.4 g/mol)

Waited for 4 hours

### Cooling to Wet Milling Conditions

Cooled to 20° C. at 0.2° C./min

Waited for 1 hour

Wet milled the suspension with high shear mill, configuration 2P-4M for 60 min (CDMP scale)

Heated reactor to 35° C. at 0.2° C./min

Held for 30 minutes

Cooled to 20° C. at 0.2° C./min

Heated reactor to 40° C. at 0.2° C./min

Held for 30 minutes

Cooled to 20° C. at 0.2° C./min

### Final Cooling and Filtration

Cooled to 5° C. at 0.2° C./min

Held at 5° C. for >3 hours before filtration—HOLD Point

### Cake Washing and Drying

Washed wet cake with isopropanol: 0.54 L Isopropanol/mol JNJ-42756493-AFK (erdafitinib mono L-lactate salt).

Washed via reactor to slightly precool the wash solvent

Washed as soon as possible after mother liquor removal

Dried solids in vacuum oven at 40° C. for >24 hours.

### Procedure 2

TABLE 15
Material list:

| Material | MW | Ratio | |
|---|---|---|---|
| JNJ-42756493-AAA | 446.55 | 1 | mol |
| (erdafitinib base) | | | |
| L-lactic acid | 90.08 | 1.02 | mol/mol |
| Isopropanol (IPA) | 60.1 | 1.006 | L/mol |
| Water | 18.02 | 0.11178 | L/mol |
| Isopropanol (IPA) | 60.1 | 1.1178 | L/mol |
| JNJ-42756493-AFK (micronized) | 536.63 | 24.39 | g/mol |
| Isopropanol (IPA) | 60.1 | 0.54 | L/mol |

### Procedure:

### Dissolve API

Added erdafitinib base to reactor

Added 1.02 mol L-lactic acid/mol erdafitinib base

Added 1.006 L Isopropanol/mol erdafitinib base to the reactor

Added 0.11178 L water/mol erdafitinib base to the reactor

Dissolved at 70° C. and hold. Visually confirmed complete dissolution

Set receiver vessel to jacket temperature 80° C.

Performed polish filtration and kept polish filter at least at 70° C.

Rinsed filter with 1.1178 L Isopropanol/mol erdafitinib base

Waited for until temperature in receiver reactor to stabilize to 80° C.

### Establish Seeding Conditions

Cooled to 63° C. at 0.3° C./min

Waited until temperature stabilized

Seeded with 4.5 m % micronized seed material (24.39 g/mol).

Waited for 4 hours

### Final Cooling and Filtration

Cooled to 5° C. at 0.2° C./min

Held at 5° C. for >3 hours before filtration—HOLD Point

### Cake Washing and Drying

Washed wet cake with Isopropanol: 0.54 L Isopropanol/mol JNJ-42756493-AFK (erdafitinib mono L-lactate).

Washed via reactor to slightly precool the wash solvent

Washed as soon as possible after mother liquor removal

Dried solids in vacuum oven at 40° C. for >24 hours.

### Example 12: Minitablets with Erdafitinib Lactate

### Manufacturing Process

The manufacturing process of the minitablets can be described as follows: First, a binder solution was created by dissolving hydroxypropyl methyl cellulose 2910 (HPMC) in purified water until a clear solution was obtained without lumps. Then, screened drug substance was transferred into the granulator and fluid bed granulation (FBG) was performed: the content was warmed up while fluidizing, the complete binder solution was sprayed upon the ingredients, and finally the granulate was dried after spraying while fluidizing. After FBG, the dried granules were screened using a suitable screen. Subsequently, the screened extragranular excipients were added to the granulate and blended to a homogeneous blend for 10 minutes. Then, screened magnesium stearate was added to the blend and blended for 5 minutes. The final blend was then compressed into core tablets using a suitable tablet press, and the tablets were passed through a deduster and metal detector.

### DoE Study:

A Design of Experiments study was performed A main compression force of 10 kN was used for the manufacturing of the DoE-concepts. The hardness, weight variation (% RSD weight, correlated with tablet defects) and ejection force of the different concepts was studied.

From this DoE study, it can be concluded that: 1) SMCC was preferred as a filler, 2) PVP VA, colloidal silicon dioxide (hydrophilic), and magnesium stearate were preferred as binder, glidant and lubricant, respectively; 3) MCC was preferred as an additional filler/binder.

TABLE 16
Exemplary minitablet compositions:

| | | | | | | |
|---|---|---|---|---|---|---|
| Component | Function | Concept 1 | Concept 2 | Concept 3 | Concept 4 | Concept 5 |

Intragranular (wt %)

| | | | | | | |
|---|---|---|---|---|---|---|
| JNJ-42756493-AFK | Drug | 70.00 | 70.00 | 70.00 | 60.00 | 80.00 |
| | Substance | | | | | |
| Hydroxypropyl Methyl | Binder | 1.43 | 1.43 | 1.43 | 1.23 | 1.63 |
| Cellulose 2910 (HPMC) | | | | | | |

Extragranular (wt %)

| | | | | | | |
|---|---|---|---|---|---|---|
| Microcrystalline Cellulose | Filler | 9.30 | 12.91 | 12.91 | 18.01 | 7.81 |
| (MCC) | | | | | | |
| Silicified Microcrystalline | Filler | 7.22 | — | — | — | — |
| Cellulose (SMCC) | | | | | | |
| Vinyl Pyrrolidone-viny1 | Binder | 9.30 | 12.91 | 12.91 | 18.01 | 7.81 |
| acetate (PVP VA) | | | | | | |
| Colloidal Silicon Dioxide | Glidant | 0.25 | 0.25 | 0.25 | 0.25 | 0.25 |
| (Hydrophilic) | | | | | | |
| Magnesium Stearate | Lubricant | 2.50 | 2.50 | — | 2.50 | 2.50 |
| Sodium Stearyl Fumarate | Lubricant | — | — | 2.50 | — | — |
| Total (weight %) | | 100.00 | 100.00 | 100.00 | 100.00 | 100.00 |

TABLE 171
IPC Results of DoE Concepts 1-3.

| | | | |
|---|---|---|---|
| Weight | Thickness | Hardness | Diameter |
| (mg) | (mm) | (N) | (mm) |

Concept 1

| | | | | |
|---|---|---|---|---|
| Average | 22.2 | 3.38 | 217 | 2.67 |
| SD | 0.62 | 0.05 | 17 | 0.01 |
| % RSD | 2.79 | 1.49 | 7.97 | 0.27 |

Concept 2

| | | | | |
|---|---|---|---|---|
| Average | 23.0 | 3.49 | 208 | 2.66 |
| SD | 0.9 | 0.10 | 17 | 0.01 |
| % RSD | 3.92 | 2.76 | 8.41 | 0.30 |

Concept 3

| | | | | |
|---|---|---|---|---|
| Average | 22.4 | 3.38 | 194 | 2.66 |
| SD | 0.6 | 0.10 | 21 | 0.00 |
| % RSD | 2.84 | 2.95 | 11.06 | 0.18 |

When evaluating the IPC results, similar values were observed for the different concepts. A comparison of hardness versus ejection force was performed. A higher standard deviation was observed for ejection force when using SSF as lubricant. Both hardness and ejection forces were comparable for Concepts 1 and 2, with slightly better results for Concept 1 (i.e., lower ejection forces and better tablet hardness). The hardness in function of compression force for each concept was investigated. The results demonstrated that the manufacturing process of concepts 1 and 2 were very robust. Promising results were also obtained for concept 4. For concept 4, slight flashing was observed at higher compression force. The compression profile of concept 5 showed comparable results to concept 2, but more variation in the data and visually less optimal tablets were observed for concept 5. Overall, concept 2 was preferred because it was the most robust concept towards manufacturability and upscaling

Many modifications and other implementations of the disclosure set forth herein will be apparent having the benefit of the teachings presented in the foregoing descriptions and the associated drawings. Therefore, it is to be understood that the disclosure is not to be limited to the specific implementations disclosed and that modifications and other implementations are intended to be included within the scope of the appended claims. Although specific terms are employed herein, they are used in a generic and descriptive sense only and not for purposes of limitation.

1. An intravesical drug delivery system comprising:
an elongated body configured for intravesical insertion into a patient; and
a drug formulation disposed in the elongated body, the drug formulation comprising an L-lactate salt of erdafitinib (N-(3,5-dimethoxyphenyl)-N′-(1-methylethyl)-N-[3-(1-methyl-1H-pyrazol-4-yl)quinoxalin-6-yl]ethane-1,2-diamine),
wherein the drug delivery system is configured to release the erdafitinib from one or more openings in the elongated body, driven by osmotic pressure.

2. The drug delivery system of claim 1, wherein erdafitinib is present as a mono L-lactate salt.

3. (canceled)

4. The drug delivery system of claim 1, wherein the elongated body comprises silicone or a thermoplastic polyurethane.

5-6. (canceled)

7. The drug delivery system of claim 1, wherein at least one of the one or more openings in the elongated body is located in a sidewall of the elongated body.

8. The drug delivery system of claim 1, wherein at least one of the one or more openings in the elongated body is located at a first end and/or at an opposing second end of the elongated body.

9. The drug delivery system of claim 1, having a single opening, which is located in a sidewall of the elongated body at position between a first end and an opposing second end of the elongated body.

10. The drug delivery system of claim 1, wherein the one or more openings has a diameter of between about 100 μm and about 200 μm.

11-33. (canceled)

34. The drug delivery system of claim 1, wherein the erdafitinib L-lactate salt is present in the drug formulation in a concentration of from 60 wt % to 91 wt %.

35. The drug delivery system of claim 34, wherein the erdafitinib L-lactate salt is present in the drug formulation in a concentration of 70 wt %.

36. The drug delivery system of claim 1, wherein the drug formulation is in the form of a plurality of tablets.

37. The drug delivery system of claim 36, wherein the drug formulation is in the form of between about 10 and about 100 mini-tablets.

38. The drug delivery system of claim 36, wherein (a) the formulation comprises a total length of from about 14.5 cm to about 15 cm of mini-tablets, and/or (b) the formulation comprises from about 920 mg to about 965 mg of mini-tablets, or from about 920 mg to about 950 mg of mini-tablets.

39. (canceled)

40. The drug delivery system of claim 1, wherein the elongated body comprises an annular wall structure defining a drug reservoir lumen in which the drug formulation is disposed.

41-45. (canceled)

46. The drug delivery system of claim 1, wherein the system is configured to release the erdafitinib at an average rate of 1 mg/day to 10 mg/day.

47. (canceled)

48. The drug delivery system of claim 1, wherein the system is configured to release the erdafitinib at an average rate of 2 mg/day to 4 mg/day.

49-50. (canceled)

51. The drug delivery system of claim 1, wherein the system is configured to release the erdafitinib with a zero order release profile.

52. (canceled)

53. The drug delivery system of claim 48, wherein the system is configured to release the erdafitinib for up to about 90 days.

54. The drug delivery system of claim 1, wherein the system comprises 500 mg of the erdafitinib (free base equivalent).

55. The drug delivery system of claim 1, wherein the system is elastically deformable between a relatively straightened deployment shape suited for insertion through the urethra of a patient and into the patient's bladder and a retention shape suited to retain the system within the bladder.

56. (canceled)

57. The drug delivery system of claim 1, wherein the system comprises an elastically deformable elongated body having two opposing free ends which lie within the boundaries of a bi-oval-like expanded retention shape.

58. The drug delivery system of claim 1, wherein the elongated body further comprises a retention frame lumen.

59. The drug delivery system of claim 58, further comprising a nitinol wire disposed in the retention frame lumen.

60. A compound, which is erdafitinib lactate salt.

61. (canceled)

62. The compound of claim 60, which is erdafitinib mono-L-lactate salt.

63. A pharmaceutical composition comprising erdafitinib L-lactate, and one or more pharmaceutical excipients.

64. (canceled)

65. The pharmaceutical composition of claim 63, wherein the at least one pharmaceutical excipient comprises or is selected from a binder, a diluent, a glidant, a lubricant, or any combination thereof.

66. The pharmaceutical composition of claim 65, wherein the binder comprises hydroxypropyl methyl cellulose, hydroxypropyl cellulose, polyvinylpyrrolidone (PVP), vinylpyrrolidone-vinyl acetate (PVP-VA), or a combination thereof.

67. The pharmaceutical composition of claim 65, wherein the binder is present in the drug formulation at a total concentration of between about 1 wt % and about 30 wt %.

68-74. (canceled)

75. The pharmaceutical composition of claim 65, wherein the drug formulation comprises an intragranular composition comprising a lactate salt of erdafitinib, and at least one intragranular excipient; and an extragranular composition comprising at least one extragranular excipient.

76. (canceled)

77. The pharmaceutical composition of claim 75, wherein the at least one intragranular excipient comprises an intragranular binder.

78. The pharmaceutical composition of claim 77, wherein the intragranular binder comprises hydroxypropyl methyl cellulose.

79. The pharmaceutical composition of claim 75, wherein the at least one extragranular excipient comprises one or more of an extragranular binder, extragranular filler (diluent), extragranular glidant, and extragranular lubricant.

80. The pharmaceutical composition of claim 79, wherein the extragranular binder comprises vinylpyrrolidone-vinyl acetate.

81-84. (canceled)

85. The pharmaceutical composition of claim 63, wherein the erdafitinib L-lactate salt is present in the drug formulation in a concentration of from 60 wt % to 91 wt %.

86. (canceled)

87. The pharmaceutical composition of claim 63, wherein the composition is in the form of a tablet.

88-92. (canceled)

93. A process for making a pharmaceutical composition in the form of a tablet, comprising:
(a) preparing an intragranular solid composition comprising:
(i) erdafitinib L-lactate; and
(ii) at least one intragranular pharmaceutical excipient;
(b) combining the intragranular solid composition with at least one extragranular pharmaceutical excipient to form a blend; and
(c) tableting the blend to form the solid pharmaceutical composition.

94-109. (canceled)

110. The drug delivery system of claim 40, wherein the elongated body is configured to release the erdafitinib through a single aperture in the annular wall structure and through microchannels transiently formed at at least one end region of the annular wall structure.

111. (canceled)

112. The drug delivery system of claim 110, wherein the microchannels transiently form when the osmotic pressure increases above a certain threshold.
